(19) United States
(12) Patent Application Publication
(10) Pub. No.: US 2022/0152188 A1
(43) Pub. Date: May 19, 2022

(54) TLR4-TLR7 LIGAND FORMULATIONS AS VACCINE ADJUVANTS

(71) Applicant: The Regents of the University of California, Oakland, CA (US)

(72) Inventors: Dennis A. Carson, La Jolla, CA (US); Howard B. Cottam, La Jolla, CA (US); Tomoko Hayashi, San Diego, CA (US); Mary Patricia Corr, San Diego, CA (US)

(21) Appl. No.: 17/438,934

(22) PCT Filed: Mar. 13, 2020
(86) PCT No.: PCT/US2020/022786

§ 371 Date: Sep. 13, 2021

Related U.S. Application Data
(60) Provisional application No. 62/818,517, filed on Mar. 14, 2019.

(51) Int. Cl. A61K 39/12; A61K 39/39; C12N 7/00

(52) U.S. Cl. CPC A61K 39/12; A61K 2039/5252; C12N 7/00; A61K 39/39

(57) ABSTRACT

A method to enhance an immune response in a mammal, and a composition comprising liposomes, a TLR4 agonist and a TLR7 agonist, are provided.

### CROSS-REFERENCE TO RELATED APPLICATIONS

This application claims the benefit of the filing date of U.S. application No. 62/818,517, filed on Mar. 14, 2019, the disclosure of which is incorporated by reference herein.

### STATEMENT OF GOVERNMENT RIGHTS

This invention was made with government support under grant number HHSN272200900034C awarded by the National Institutes of Health. The government has certain rights in the invention.

### BACKGROUND

The most effective way to protect individuals from the insidious threat of many infectious diseases is through vaccination. Effective vaccination requires the use of antigens that can elicit an immune response in the host capable of providing subsequent protection against that particular infectious agent for which the vaccine is specific. Thus, the vaccine antigen must be immunogenic enough to induce a level of immune response—humoral and/or cell-mediated—to be protective in the host. An infectious agent of global concern is influenza virus. Seasonal influenza viruses cause annual epidemics that lead to 250-500,000 deaths worldwide (WHO), more than 80,000 deaths in the U.S. alone last winter. In addition, new pandemics emerge occasionally that have caused several million deaths—posing very real global threats. Particularly vulnerable to these threats are high-risk populations, such as the elderly, newborns, and immune compromised individuals. Vaccination against seasonal influenza can be moderately effective if matched to the circulating virus strain of the season. However, since influenza viruses are constantly undergoing change (antigenic drift), it is difficult to predict what subtype and strain of virus will be circulating in the next influenza season or in the next pandemic, and to allow sufficient time (about 6 months) for manufacture and distribution of conventional vaccines.

These conventional vaccines are typically based on antigens associated with the influenza hemagglutinin (HA) protein, and in particular, the globular head domain of the protein. This highly immunogenic head domain is variable across strains and subtypes of influenza viruses and thus, an immune response against one globular head domain subtype might be limited to that particular head domain and fail to provide an adequate immune response against a virus strain having a different head domain. Influenza HA antigens derived from the stem or stalk domain of the protein, which are more highly conserved across virus strains, are generally much less immunogenic than the head domain antigens that are typically dominant in the conventional vaccines and therefore there is a need to augment the immunogenicity of these HA stalk antigens to a level that would generate an adequate immune response in the host, resulting in an immune response against multiple influenza strains.

### SUMMARY

The successful use of suitable adjuvant combination formulations in vaccines against globally important infectious agents such as influenza virus potentially represents a major step forward in medicine to broaden and enhance the protection of individuals from the ever-changing threats of these viral pathogens.

This disclosure provides for formulating a combination of a TLR4 agonist with a TLR7 agonist as adjuvant in the same liposomal nanoparticles provides several advantages over mixed combinations of the separate formulated and non-formulated agonists. The formulated combinations may have a certain ratio of TLR4 to TLR7 in the nanoparticles for desired immunoactivity. Each compound was formulated alone and in combination based on data generated with various combination ratios of compounds. Formulated versus unformulated combinations, mixed and combined in the same particles were compared side-by-side. The results of the immunization studies showed that certain ratios of combined compounds in liposomes provided greater and broader immunoactivity than either compound alone and that formulated was better than unformulated combinations. Antigens used were OVA and inactivated influenza virus.

As disclosed herein, 2B182C (an exemplary TLR4 agonist) and 1V270 (an exemplary TLR7 agonist) were formulated together in one formulation and immunization studies conducted in mice. Each compound was formulated alone and in combination based on data generated with various combination ratios of compounds. Formulated versus unformulated combinations, and mixed and combined in the same particles, were compared side-by-side. The results of the immunization studies showed that a particular ratio of combined compounds in liposomes provided greater and broader immunoactivity than either compound alone and that formulated was better than unformulated combinations. Antigens used were OVA and inactivated influenza virus.

In one embodiment, the disclosure provides for a method to enhance an

immune response in a
mammal, comprising administering to a mammal in need thereof a TLR4 agonist and a TLR7 agonist in an
effective amount. In one embodiment, the TLR4 agonist and a TLR7 agonist are administered
simultaneously. In one embodiment, the TLR4 agonist and a TLR7 agonist are administered in a liposomal
formulation. In one embodiment, the TLR4 agonist and a TLR7 agonist are in separate liposomal
formulations. In one embodiment, the TLR4 agonist has formula (II). In one embodiment, the TLR7 agonist
has formula (I). In one embodiment, one or more immunogens (antigens) are also administered,
e.g., at the same time as the adjuvants and optionally in the same formulation as the adjuvants. In one
embodiment, the immunogen is a microbial immunogen. In one embodiment, the microbe is a virus, such as
influenza or varicella, or a bacteria. In one embodiment, the mammal is a human. In one embodiment, the
amount of the TLR7 agonist is about 0.01 to 100 nmol, about 0.1 to 10 nmol, or about 100 nmol to about 1000
nmol. In one embodiment, the amount of the TLR4 agonist is about 2 to 20 umol, about 20 nmol to 2 umol, or
about 2 umol to about 100 umol. In one embodiment, the ratio of TLR7 to TLR4 agonist is about 1:10, 1:100,
1:200, 5:20, 5:100, or 5:200. In one embodiment, the formulation is injected. In one embodiment, the
liposomal formulation comprises DOPC, cholesterol or combinations thereof.

Also provided are pharmaceutical formulations comprising liposomes, a TLR4

agonist and a TLR7
agonist, e.g., where the liposome comprises DOPC, cholesterol or combinations thereof, where in one
embodiment, the amount of the TLR7 agonist is about 0.01 to 100 nmol, about 0.1 to 10 nmol, or about 100
nmol to about 1000 nmol; where the amount of the TLR4 agonist is about 2 nmol to 20 umol, about 20 nmol to
2 umol, or about 2 umol to about 100 umol; or wherein the ratio of TLR7 to TLR4 agonist is about 1:10, 1:100,
1:200, 5:20, 5:100, or 5:200.

### BRIEF DESCRIPTION OF FIGURES

FIG. 1. Exemplary liposomal formulations.

FIG. 2. In vitro immunostimulatory activity of 1V270 (1 μM), 2B182C (200 μM), or combination of 1V270 (1 μM) and 2B182C (200 mM) in DMSO or liposomal formulation.

Murine bone marrow derived dendritic cells from wild type C57BL/6 mice were incubated with 1V270 (1 μM), 2B182C (200 μM), or combination of 1V270 (1 μM) and 2B182C (200 mM) in DMSO or liposomal formulation for 18 h. IL-6 release in the culture supernatant was measured by ELISA.

FIG. 3. Liposomal formulation mitigates TLR4 independent cytotoxicity Murine bone marrow derived dendritic cells from wild type C57BL/6 mice or TLR4 deficient mice (C57BL/6 background) were incubated with 1V270 (1 μM), 2B182C (200 μM), or combination of 1V270 (1 μM) and 2B182C (200 mM) in DMSO or liposomal formulation for 18 h. Cell viability was assessed by MTT assay.

FIG. 4. Exemplary experimental protocol.

FIG. 5. Anti-HA IgG1 and IgG2a levels for formulation.

FIG. 6. Ratio of IgG2a/IgG1 for formulation.

FIG. 7. Gating strategy for GC cells and plasmablasts.

FIG. 8. Cells types induced by administration of 1V270 and/or 2B182c or AddaVax. The combination with formulated 1V270 and 2B182c significantly increased number of GC B cells and plasmablasts.

FIG. 9. Anti-HA IgG levels as measured by ELISA or BCR-seq induced by administration of 1V270 and/or 2B182c or AddaVax. IgG2a production was strongly increased by combination treatment and Th1 responses was induced by formulated 2B182c.

FIG. 10. BCR diversity was increased by combination treatment.

FIG. 11. TCR clonality after administration of antigen and 1V270 and/or 2B182c or AddaVax. TCR clonality was increased after 2B182c treatment and Addavax.

FIG. 12. BCR diversity and TCR clonality after administration of 1V270 and/or 2B182c or AddaVax. BCR diversity was increased by combination treatment and TCR clonality was increased after 2B182c treatment and Addavax.

FIG. 13. Clonal similarity.

FIG. 14. Shared clones.

FIG. 15. Cluster analysis.

FIG. 16. Number of clusters with similar sequence as known antibody against influenza.

FIG. 17. Cytotoxicity and IL-12 secretion analysis. Liposomal adjuvants induced IL-12 secretion with lower cytotoxicity in BMDC.

FIG. 18. Anti-NAIgG1 and IgG2a analyses after administration of 1V270 and/or 2B182c (unformulated and formulated) or AddaVax.

FIGS. 19A-19B. Liposomal formulation of 2B182c and 1V270 skews immune response toward Th1 response. (A) BALB/c mice were immunized with inactivated Cal 2009 H1N1 influenza virus (10 μg/injection) mixed with TLR4 ligand and/or TLR7 ligand in DMSO (D) or liposomal (L) formulation on days 0 and 28. The sera were collected on day 28 and HA or NA specific IgG1 and IgG2a were determined by ELISA. (B) Th1/TH2 balance was evaluated by IgG2a/IgG1 ratio. *P<0.05, **P<0.001 by Mann-Whitney test.

FIGS. 20A-20C. Number of germinal center B cells and plasmablasts in the draining lymph nodes are increased by combination adjuvant treatment with liposomal 2B182c and 1V270. (A) Experimental protocol. (B) Gating strategy for the flow cytometory data. (C) Total numbers of B cells, germinal center B cells (CD3⁻CD19⁺CD95⁺GL7⁺) and plasmablasts (CD3⁻CD19⁺CD138⁺) were calculated. BL; blank liposomes. *:p<0.05, * ; p<0.01, **:p<0.001 by Kruskal-Wallis test with Dunn's post hoc test, compared to antigen+BL.

FIGS. 21A-21B. 2B182C is effective on both human (A) and mouse (B) TLR4 with lower concentration. HEK TLR reporter cells (HEK-Blue™ hTLR4 and HEK-Blue™ mTLR4) were treated with compounds 1Z105 and 2B182C (2-fold serial dilution from 10 μM) for 20 h. NF-kB inducible NF-kB SEAP levels in the culture supernatant were evaluated according to manufacturers protocol.

FIGS. 22A-22C. 200 nmol/injection 2B182c induced higher level of antigen specific IgG1 and anti-NA IgG2a. (A) Experimental protocol for comparison of two TLR agonists 1Z105 and 2B182C. BALB/c mice (n=5/group) were i.m. immunized with IIAV (10 μg/injection) plus TLR4 agonists 1Z105 or 2B182C (40 and 200 nmol/injection) in both hind legs on days 0 and 21, were bled on day 28, and sera were evaluated for antibodies against hemagglutinin (HA) and neuraminidase (NA) by ELISA. 10% DMSO was used as vehicle. (B) anti-HA and -NA IgG1 antibodies. (C) anti-HA and -NA IgG2a antibodies. In each box plot, the line within the box represents the median, the bounds are the upper and lower quartiles and the bars indicate minimum and maximum values. *P<0.05, *P<0.01, Kruskal-Wallis test with Dunn's post hoc test (vs. antigen+vehicle).

FIGS. 23A-23C. Combination with 2B182C and TLR7 agonist 1V270 increased both antigen specific IgG1 and IgG2a. (A-C) BALB/c mice (n=5-6) were immunized with IIAV and adjuvants as shown in FIG. 2A. AddaVax™, which is similar formulation as MF59 was used as a positive control. anti-HA and -NA IgG1 (A), anti-HA and -NA IgG2a productions (B) were determined by ELISA. In each box plot, the line within the box represents the median, the bounds are the upper and lower quartiles and the bars indicate minimum and maximum values. *P<0.05, *P<0.01, **P<0.001, Kruskal-Wallis test with Dunn's post hoc test. Four groups except No antigen and AddaVax were compared (all pairs). (C) anti-HA IgG1 and IgG2a levels induced by all combination treatment (normalized to vehicle) are shown by mean of 5-11 mice/group. Each dot indicates individual animal. A solid line in black indicates IgG2a/IgG1=1. All animals immunized with combination with 1V270 and 2B182C distributed above IgG2a/IgG1=1, suggesting that the immune balance in these mice were biased toward Th1 immune response.

FIGS. 24A-24B. Antigen specific IgM productions on day 28. (A and B) BALB/c mice (n=5-6) were immunized with IIAV (10 μg/injection) and indicated adjuvants as shown in FIG. 2A. Antigen specific IgM level was measured by ELISA. (A) anti-HA and -NA IgM production induced by TLR4 agonists 1Z105 or 2B182C (40 and 200 nmol/injection). (B) Combination of TLR7 agonist 1V270 (1 nmol/injection) and TLR4 agonists 1Z105 or 2B182C (200 nmol/injection) showed minimal effects on antigen specific IgM induction. *P<0.05, Kruskal-Wallis test with Dunn's post hoc test.

FIGS. 25A-25B. Liposomal 1V270 and 2B182C induced similar level of IL-12 release with less cytotoxicity. (A) IL-12 secretion level. (B) % viability. Muse primary BMDCs were treated with 1V270 (0.0625 uM) and 2B182C (12.5 uM). 1V270/2B182c ratio was kept as 1 to 200, which was determined as the best ratio in FIG. 3. After overnight incubation, IL-12 level in the culture supernatant was examined by ELISA and cell viability was evaluated by MTT assay. *P<0.05, *P<0.01, One-tailed unpaired t-test with Welch's correction, DMSO formulation (D) vs liposomal formulation (L) in each compound.

FIG. 25C. Histologic analysis of local immune cell infiltration following injection with the combination adjuvants. BALB/c mice were intramuscularly injected with liposomal formulation of 1V270 (1 nmol/injection), 2B182C (200 nmol/injection), or combination of 1V270 (1 nmol/injection) and 2B182C (200 nmol/injection). The tissues were collected, fixed, and embedded in paraffin block. 10 μm section were stained with H&E. Low and high magnifications were obtained using 20× and 40× objective lenses, respectively. Scale bars in low and high magnification image indicate 50 m and 20 μm, respectively.

FIG. 25D. BALB/c mice (n=5/group) were i.m. injected with vehicle, 1V270, 2B182C, 1V270+2B182C with DMSO formulation or liposomal formulation [1 nmol/injection 1V270 and 200 nmol/injection 2B182C in a volume of 50 μL]. AddaVax™ (25 μL/injection) was used as a positive control. Two and 24 h later, sera were collected and examined for IL-12p40, TNF and KC secretion by Luminex multiplex cytokine assay (A). Data shown are means±SEM. *P<0.05, **P<0.01, Two-tailed Mann-Whitney U test. +P<0.05, ++P<0.01, Kruskal-Wallis with Dunn's post hoc test to compare 4 groups (vehicle, 1V270, 2B182C, 1V270+2B182C in the same formulation).

FIGS. 26A-26D. Liposomal 1V270 and 2B182C synergistically enhanced anti-HA and anti-NA IgG1 and IgG2a production. (A-C) BALB/c mice (n=5/group) were i.m. immunized on days 0 and 21 with IIAV (10 μg/injection) with formulated adjuvants as shown in FIG. 22A. Liposomal TLR7 agonist 1V270 (lipo-1V270, 1 nmol/injection), liposomal TLR4 agonist 2B182C (lipo-2B182C, 200 nmol/injection) and liposomal combined adjuvants of 1V270 and 2B182C (lipo-1V270+2B182C, 1 nmol/injection+200 nmol/injection) were injected. Vehicle is 1,2-dioleoyl-sn-glycero-3-phosphocholine and cholesterol (DOPC/Chol, control liposomes). AddaVax™ was used as a positive control. Sera were collected on day 28 and HA or NA specific IgG1. IgG2a and total IgG were determined by ELISA. *P<0.05 and **P<0.01, Kruskal-Wallis test with Dunn's post hoc test. Four groups except No antigen and AddaVax were compared (all pairs). Data are representative of two independent experiments with similar results.

FIG. 27. antigen specific IgM level induced by formulated adjuvant. BALB/c mice (n=5/group) were i.m. immunized on days 0 and 21 with IIAV (10 μg/injection) with formulated adjuvants as shown in FIG. 2A. Liposomal TLR7 agonist 1V270 (lipo-1V270, 1 nmol/injection), liposomal TLR4 agonist 2B182C (lipo-2B182C, 200 nmol/injection) and combined liposomal adjuvants of 1V270 and 2B182C (lipo-1V270+2B182C, 1 nmol/injection+200 nmol/injection) were injected. Vehicle is 1,2-dioleoyl-sn-glycero-3-phosphocholine and cholesterol (DOPC/Chol, control liposomes). AddaVax™ was used as a positive control. The sera were collected on day 28 and examined for HA or NA specific IgM. *P<0.05, Kruskal-Wallis test with Dunn's post hoc test. Four treatments except no antigen and AddaVax were compared (all pairs). Data are representative of two independent experiments with similar results.

FIGS. 28A-28C. Formulated combined adjuvants increased Tfh and antibody secreting cells. (A) BALB/c mice (n=4-5/group) were vaccinated on days 0 and 21 with IIAV (10 μg/injection) with 1V270 (1 nmol/injection) and/or 2B182C (200 nmol/injection) in a total volume of 50 μL. Twenty-eight days later, lymphocytes in inguinal lymph nodes were harvested for FACS analysis. Gating strategy for Tfh cells (CD3+CD4+PD-1+CXCR5+), GC B cells (CD3− CD19+CD95+GL7+), Plasmablasts (CD3− CD19+CD138+) and plasma cells (CD3− CD19− CD138+) is shown. (B) % Tfh cells, GC B cells, plasmablasts and plasma cells in live cells. Bars indicates mean±SEM. *P<0.05, *P<0.01, Kruskal-Wallis with Dunn's post hoc test. Four conditions except AddaVax were compared (all pairs).

FIGS. 29A-29B. Formulated combined adjuvants increased Tfh and antibody secreting cells. BALB/c mice (n=4-5/group) were vaccinated on days 0 and 21 with IIAV (10 μg/injection) with 1V270 (1 nmol/injection) and/or 2B182C (200 nmol/injection) in a total volume of 50 μL. Twenty-eight days later, lymphocytes in inguinal lymph nodes were harvested for FACS analysis (FIG. 5A). Gating strategy for Tfh cells (CD3+CD4+PD-1+CXCR5+), GC B cells (CD3− CD19+CD95+GL7+), Plasmablasts (CD3− CD19+CD138+) and plasma cells (CD3− CD19− CD138+) is shown in FIG. 5B. (A) Number of Tfh cells, GC B cells, plasmablasts, plasma cells. (B) Number of total cells. Bars indicates mean±SEM. *P<0.05, **P<0.01, Kruskal-Wallis with Dunn's post hoc test (all pairs).

FIGS. 30A-30C. Formulated combination of 1V270 and 2B182C. (A and B) BALB/c mice were vaccinated on days 0 and 21 with IIAV with formulated adjuvants and inguinal lymph nodes were harvested on day 28 for BCR repertoire analysis. (A) BCR diversity of total IGH, IGHG1 and IGHG2A. (B) Similarity analysis. Jaccard indices are shown. (C) TCR clonalities indicated by “1-pielou's index” for TCRα and TCRβ. Bars indicates mean±SEM. *P<0.05, **P<0.01, Kruskal-Wallis with Dunn's post hoc test (vs. liposomes).

FIGS. 31A-311. Lipo-2B182C and lipo-1V270+2B182C protect mice against homologous influenza virus. (A) Experimental schedule of homologous influenza virus challenge. (B) Mean body weight change indicated by % initial body weight. *P<0.05, **P<0.01, One-way ANOVA with Dunnett's post hoc test. (C) Survival rate of mice post challenge with homologous virus (H1N1pdm). Kaplan-Meier curves with Log-rank test are shown. Lung virus titer (D) and cytokine level in lung fluids (E) were evaluated. Lung lavage was performed on days 3 and 6. **P<0.01, Kruskal-Wallis with Dunn's post hoc test (vs. liposomes). (F) Relationship between lung virus titers and pro-inflammatory cytokines, MCP-1 (left) and IL-6 (right). Spearman rank correlation test, (MCP-1; **P<0.0001, Spearman r=0.83, IL-6; ***P<0.0001, Spearman r=0.79). HI titers (G) and VN titers against homologous virus (H). *P<0.01, ***P<0.001, Kruskal-Wallis with Dunn's post hoc test (all pairs). (1) Relationship between VN titers and lung virus titer. Each dot indicates a VN titer and a lung virus titer in the same animal. **P<0.01. Spearman rank correlation test, Spearman r=−0.59.

FIGS. 32A-32C. Heterologous challenge with H3N2 virus. BALB/c mice were immunized with formulated adjuvants plus IIAV (H1N1) as described in FIG. 31A and intranasally challenged with heterologous virus A/Victoria3/75 (H3N2). (A) Body weight loss were monitored. No significance was detected by One-way ANOVA. (B) Survival rate of mice post challenge with heterologous virus. Kaplan-Meier curves with Log-rank test (n.s.) are shown. (C) Lung virus titers on days 3 and 6. No significance was detected by Kruskal-Wallis test.

FIGS. 33A-33G. A and E) protocols. B-C and F-G) Body weight and survival overtime after infection with A/PuertoRico/8/1934 or B/Florida/04/ in mice administered 1V270 and/or 2B182c or AddaVax. D) IgG2a/IgG1 ratio in mice administered 1V270 and/or 2B182c or AddaVax.

FIGS. 34A-34B. A) Anti-HA IgG1, anti-HA IgG2a and anti-HA IgM in mice administered 1V270, 1Z105, 2B182c or AddaVax. B) Anti-NA IgG1, anti-NA IgG2a and anti-NA IgM in mice administered 1V270, 1Z105, 2B182c or AddaVax.

FIGS. 35A-35F. A and B) Anti-HA and anti-NA IgG1, C-D) Anti-HA and anti-NA IgG2a and E-F) anti-HA and anti-NA IgM in mice administered 1V270, 1Z105, 2B182c or AddaVax. B) Anti-NA IgG1, anti-NA IgG2a and anti-NA IgM in mice administered 1V270, 1Z105, 2B182c or AddaVax.

FIGS. 36A-36B. Anti-HAIgG2a and IgG1 in mice administered different doses of 1V270, 1Z105, 2B182c, or a combination thereof.

FIG. 37. Schematic of various liposomes and exemplary protocol.

FIGS. 38A-38B. ELISA using peptide array of HA of A/California/04/2009 (H1N1)pdm. BALB/c mice (n=5-10) were immunized with IIAV plus Lipo-Veh (blank liposome), Lipo-1V270, Lipo-2B182C, Lipo-(1V270+2B182C) (co-encapsulated combination) or (Lipo-1V270)+(Lipo-2B182C) (admixed combination) on days 0 and 21, and were bled on day 28. Peptide arrays of HA of A/California/04/2009 (H1N1)pdm (NR-15433) were obtained from BEI resources. Peptides in groups of 5 were pooled and 28 peptide pools were generated. (A) Heatmap of OD_{405-570 nm} with results of ELISA. Each row and column indicate each peptide pool and mouse, respectively. (B) Statistical analysis was performed on averages of 28 peptide pools in individual mouse. *P<0.01, *P<0.0001, Kruskal-Wallis with Dunn's post hoc test. +P<0.05, Mann-Whitney test.

FIGS. 39A-39D. ELISA for cross-reactivity of antibodies. BALB/c mice (n=5/group) were immunized with IIAV plus Lipo-Veh, Lipo-1V270, Lipo-2B182C, Lipo-(1V270+2B182C), or (Lipo-1V270)+(Lipo-2B182C) on days 0 and 21, and were bled on day 28. Sera were serially diluted (1:100 to 1:409600) and assessed for total IgG levels against HAs of Puerto RicoH1N1, H11N9, H12N5, H7N7, and H3N2, and NAs of H5N1, H10N8, H3N2 and H7N7 by ELISA. (A) Phylogenetic relationship of HAs of influenza A viruses used in this study. Amino acid sequences of proteins used in ELISA were aligned by MUSCLE algorithm using Influenza Research Database (https://www.fludb.org/brc/home.spg?decorator=influenza). Phylogenetic tree was constructed by Neighbor-joining method using MEGAX software (https://www.megasoftware.net/). (B) Total IgG titer curves for HAs of H1N1, H11N9, H12N5, H3N2 and H7N7. (C) Phylogenetic relationship of NAs. (D) Total IgG titer curves for NAs of H5N1, H10N8, H3N2, and H7N7. Sera were 1:4 diluted from starting from ×100 to 409600 and total IgG levels were evaluated by ELISA. Data shown are means±SEM.

FIGS. 40A-40B. Lipo-(1V270+2B182C) induced cross reactive antibodies. (A and B) BALB/c (n=5/group) mice were immunized with IIAV [A/California/04/2009 (H1N1)pdm09] plus Lipo-Veh, Lipo-1V270, Lipo-2B182C, Lipo-(1V270+2B182C), or (Lipo-1V270)+(Lipo-2B182C) on days 0 and 21 and were bled on day 28. Sera were serially diluted (1:100 to 1:409600) and assessed for total IgG levels against HAs of Puerto RicoH1N1, H11N9, H12N5, H7N7, and H3N2, and NAs of H5N1, H10N8, H3N2 and H7N7 by ELISA. Geometric means of total IgG titer curves of individual mice calculated using prism5 are shown. Total IgG titer curves and phylogenetic relationship of HA proteins used in this study and are shown above. *P<0.05, *P<0.01, Kruskal-Wallis with Dunn's post hoc test. +P<0.05, ++P<0.01, Mann-Whitney test.

FIG. 41. Exemplary TLR4 and TLR7 agonists.

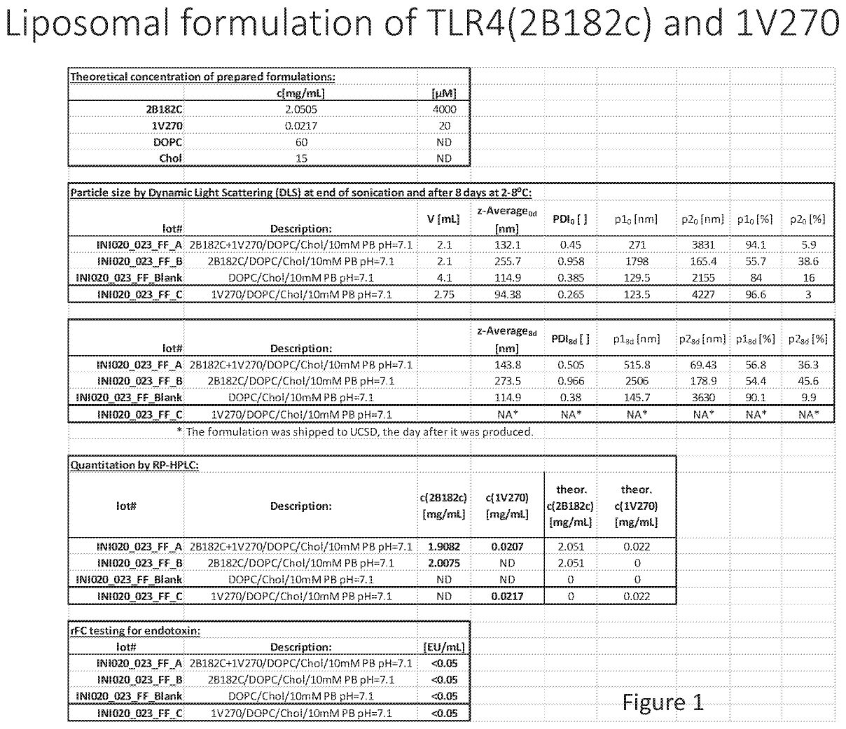

### DETAILED DESCRIPTION

The use of adjuvants in vaccines is a well-established method to promote a stronger immune response to weakly immunogenic antigens. In addition, adjuvants may also enhance and potentially broaden the immune response by promoting the immunogenicity of weakly immunogenic antigens. Only a few adjuvants are currently licensed for use in vaccines (O'Hagan, et al. doi: 10.1016/j.vaccine.2015.01.088).

Moreover, the majority of existing vaccines contain a single adjuvant and recent evidence suggests that it is unlikely to be sufficient for induction of a protective immune response against many emerging infectious diseases. (Underhill, doi: 10.1111/j.1600-065X.2007.00548.x).

The use of combinations of TLR agonists as adjuvants has often resulted in overall enhancement of immune responses but, in the case of infectious disease vaccines such as influenza, enhancement of a Th1 (cell mediated) or skewing of the response toward a Th1 type comes at the expense of the Th2 (humoral or antibody) type. Indeed, sometimes this can result in insufficient protective Th2 antibody production in spite of the increased Th1 response, and for influenza infections, a certain protective antibody titer is thought to be the major factor for providing effective protection through immunization.

In the present disclosure, the combination ratio of TLR4/TLR7 agonists in a single nanoparticle formulation was found to not only enhance the overall immune response to antigen, but also to provide sufficient protective antibody generation for effective protection against a lethal virus challenge in mice. The immunological status of humans will be quite different from that of mice, where mice are generally naive toward antigens such as influenza, whereas humans usually have been exposed to influenza antigens over many years through both natural and vaccine exposures. The same thing is true for other infectious agents such as chicken pox (varicella zoster), which can appear later in humans as shingles.

It is known that immunization with one antigen blocks robust immune responses to a second, similar antigen. This can be due to 1) epitope exclusion, where pre-existing antibodies, especially mucosal IgA, shield the vaccine from all antigen presenting cells; 2) reduced dendritic cell (DC) access, where memory B cells internalize the new vaccine, reducing DC access and activation and T cell immunization; 3) T cell competition, where memory B cells are activated, consuming cytokines, co-factors, and trapping T cells that could react with antigen loaded DCs.

The present disclosure overcomes these liabilities by 1) encapsulating the vaccine in liposomal nanoparticles that preferentially delivers the vaccine to DCs and 2) activating DCs using combination TLR agonists in a particular ratio that will increase the numbers diversity of activated T cells against the vaccine antigens. This invention discloses our discovery that formulating a combination of a TLR4 agonist with a TLR7 agonist as adjuvant in the same liposomal nanoparticles provides several advantages over mixed combinations of the separate formulated and non-formulated agonists. The formulated combinations may have a certain ratio of TLR4 to TLR7 in the nanoparticles for immunoactivity.

Advantages of these combinations at the ratio include: 1) enhanced activity vs DMSO formulations providing for greater Th1 and Th2 immune responses; 2) lower toxicity vs DMSO formulations: 3) shielding of the antigen (for vaccine use) from B-cells and from IgA of hyperimmune individuals, particularly for mucosal influenza immunization, and allow dendritic cells to present important epitopes for effective protective response; and/or 4) broaden the immune response to include response to less immunogenic antigens as in the case of the HA stalk antigens in influenza, thus resulting in a more universal vaccine.

The use of combinations of TLR agonists as adjuvants has often resulted in overall enhancement of immune responses but, in the case of infectious disease vaccines such as influenza, enhancement of a Th1 (cell mediated) or skewing of the response toward a Th1 type comes at the expense of the Th2 (humoral or antibody) type. Indeed, sometimes this can result in insufficient protective Th2 antibody production in spite of the increased Th1 response, and for influenza infections, a certain protective antibody titer is thought to be the major factor for providing effective protection through immunization.

In the present invention, the combination ratio of TLR4/TLR7 agonists in a single nanoparticle formulation was found to not only enhance the overall immune response to antigen, but also to provide sufficient protective antibody generation for effective protection against a lethal virus challenge in mice. The immunological status of humans will be quite different from that of mice, where mice are generally naive toward antigens such as influenza, whereas humans usually have been exposed to influenza antigens over many years through both natural and vaccine exposures. The same thing is true for other infectious agents such as chicken pox (varicella zoster), which can appear later in humans as shingles.

It is known that immunization with one antigen blocks robust immune responses to a second, similar antigen. This can be due to 1) epitope exclusion, where pre-existing antibodies, especially mucosal IgA, shield the vaccine from all antigen presenting cells; 2) reduced dendritic cell (DC) access, where memory B cells internalize the new vaccine, reducing DC access and activation and T cell immunization; 3) T cell competition, where memory B cells are activated, consuming cytokines, co-factors, and trapping T cells that could react with antigen loaded DCs.

The present invention overcomes these liabilities by 1) encapsulating the vaccine in liposomal nanoparticles that preferentially delivers the vaccine to DCs and 2) activating DCs using combination TLR agonists in a specific ratio that will increase the numbers diversity of activated T cells against the vaccine antigens.

### Definitions

A composition is comprised of “substantially all” of a particular compound, or a particular form a compound (e.g., an isomer) when a composition comprises at least about 90%, and at least about 95%, 99%, and 99.9%, of the particular composition on a weight basis. A composition comprises a “mixture” of compounds, or forms of the same compound, when each compound (e.g., isomer) represents at least about 10% of the composition on a weight basis. A TLR7 agonist of the invention, or a conjugate thereof, can be prepared as an acid salt or as a base salt, as well as in free acid or free base forms. In solution, certain of the compounds of the invention may exist as zwitterions, wherein counter ions are provided by the solvent molecules themselves, or from other ions dissolved or suspended in the solvent.

The term “toll-like receptor agonist” (TLR agonist) refers to a molecule that binds to a TLR. Synthetic TLR agonists are chemical compounds that are designed to bind to a TLR and activate the receptor.

Within the present invention it is to be understood that a compound of formula (I) or (II) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

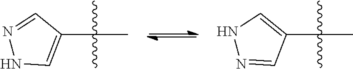 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

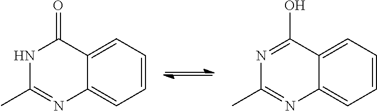

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active. i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

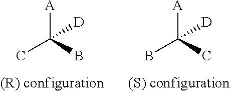

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. In one embodiment, the isolated isomer is at least about 80%, e.g., at least 90%, 98% or 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, Pa., p. 1418 (1985), the disclosure of which is hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A candidate molecule or compound described herein may be in an amount in a formulation or medicament, which is an amount that can lead to a biological effect, or lead to ameliorating, alleviating, lessening, relieving, diminishing or removing symptoms of a condition, e.g., disease, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism (microbe) in a system (e.g., cell, tissue, or subject) infected with a microbe, reducing the rate of microbial propagation, reducing the number of symptoms or an effect of a symptom associated with the microbial infection, and/or removing detectable amounts of the microbe from the system. Examples of microbe include but are not limited to virus, bacterium and fungus.

The term “therapeutically effective amount” as used herein refers to an amount of a compound, or an amount of a combination of compounds, to treat or prevent a disease or disorder, or to treat a symptom of the disease or disorder, in a subject. As used herein, the terms “subject” and “patient” generally refers to an individual who will receive or who has received treatment (e.g., administration of a compound) according to a method described herein.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

The terms “subject,” “patient” or “subject in need thereof” refers to a living organism suffering from or prone to a disease or condition that can be treated by administration of a compound, pharmaceutical composition, mixture or vaccine as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

The term “effective amount” as used herein refers to an amount effective to achieve an intended purpose. Accordingly, the terms “therapeutically effective amount” and the like refer to an amount of a compound, mixture or vaccine, or an amount of a combination thereof, to treat or prevent a disease or disorder, or to treat a symptom of the disease or disorder, in a subject in need thereof.

The term “TLR” refers to Toll-like receptors which are components of the innate immune system that regulate NFκB activation.

The terms “TLR modulator,” “TLR immunomodulator” and the like as used herein refer, in the usual and customary sense, to compounds which agonize or antagonize a Toll Like Receptor. See e.g., PCT/US2010/000369, Hennessy, E. J., et al., Nature Reviews 2010, 9:283-307; PCT/US2008/001631; PCT/US2006/032371; PCT/US2011/000757. Accordingly, a “TLR agonist” is a TLR modulator which agonizes a TLR, and a “TLR antagonist” is a TLR modulator which antagonizes a TLR.

The term “TLR4” as used herein refers to the product of the TLR4 gene, and homologs, isoforms, and functional fragments thereof: Isoform 1 (NCBI Accession NP_612564.1); Isoform 2 (NCBI Accession NP_003257.1); Isoform 3 (NCBI Accession NP_612567.1). Agonists of TLR4 that may be included in the disclosed formulations include but are not limited, a compound of formula (II), e.g., a pyrimidoindole, aminoalkyl glucosaminide phosphates, e.g., CRX-601 and CRX-547), RC-29, monophosphorul lipid A (MPL), glucopyranosyl lipid adjuvant (GLA and SLA), OM-174, PET Lipid A. ONO-4007, INI-2004 (a di-amine allose phosphate), and E6020.

The term “TLR7” as used herein refers to the product (NCBI Accession AAZ99026) of the TLR7 gene, and homologs, and functional fragments thereof. Agonists of TLR7 that may be included in the disclosed formulations include but are not limited, a compound of formula (I), imidazoquinolines, e.g., imiquimod, CL097 or gardiquimid, CL264, adenine analogs such as CL087, thiazoloquinolines such as 3M002 (CL075), guanosine analogs such asloxonbine, or thioquinoline.

### TLR4 and TLR7

Toll-like receptors (TLRs) are pattern recognition receptors that recognize conserved microbial products, known as pathogen-associated molecular patterns (PAMPs). TLR4 recognizes LPS. TLR4 signaling activates MyD88 and TRIF-dependent pathways. MyD88 pathway activates NF-κB and JNK to induce inflammatory response. TRIF pathway activates IRF3 to induce IFN-α production.

TLR4 is expressed predominately on monocytes, mature macrophages and dendritic cells, mast cells and the intestinal epithelium. TLR modulators (antagonists) for TLR4 include NI-0101 (Hennessy 2010, Id.), 1A6 (Ungaro, R., et al., Am. J. Physiol. Gastrointest. Liver Physiol. 2009, 296:G1167-G1179), AV411 (Ledeboer, A., et al., Neuron Glia Biol. 2006, 2:279-291; Ledeboer, A., et al., Expert Opin. Investig. Drugs 2007, 16:935-950), Eritoran (Mullarkey, M., et al., J. Pharmacol. Exp. Ther. 2003, 305:1093-1102), and TAK-242 (Li, M., et al., Mol. Pharmacol. 2008, 69:1288-1295). TLR modulators (agonists) for TLR4 include Pollinex® Quattro (Baldrick, P., et al., J. Appl. Toxicol. 2007, 27:399-409; DuBuske, L., et al., J. Allergy Clin. Immunol. 2009, 123:S216). TLR7 signaling activates MyD88-dependent pathway and IRF7-dependent signaling. IRF7 pathway induces IFN-α production.

TLR7 senses ss-RNA or synthetic chemicals (Imiquimod, R848). TLR7 and TLR8 are found in endosomes of monocytes and macrophages, with TLR7 also being expressed on plasmacytoid dendritic cells, and TLR8 also being expressed in mast cells. Both these receptors recognize single stranded RNA from viruses. Synthetic ligands, such as R-848 and imiquimod, can be used to activate the TLR7 and TLR8 signaling pathways. See e.g., Caron, G., et al., J. Immunol. 2005. 175:1551-1557. TLR9 is expressed in endosomes of monocytes, macrophages and plasmacytoid dendritic cells, and acts as a receptor for unmethylated CpG islands found in bacterial and viral DNA. Synthetic oligonucleotides that contain unmethylated CpG motifs are used to activate TLR9. For example, class A oligonucleotides target plasmacytoid dendritic cells and strongly induce IFNa production and antigen presenting cell maturation, while indirectly activating natural killer cells. Class B oligonucleotides target B cells and natural killer cells and induce little interferon-a (IFNa). Class C oligonucleotides target plasmacytoid dendritic cells and are potent inducers of IFNa. This class of oligonucleotides is involved in the activation and maturation of antigen presenting cells, indirectly activates natural killer cells and directly stimulates B cells. See e.g., Vollmer, J., et al., Eur. J. Immunol. 2004, 34:251-262; Strandskog, G., et al., Dev. Comp. Immunol. 2007, 31:39-51.

Reported TLR modulators (agonist) for TLR7 include ANA772 (Kronenberg, B. & Zeuzem, S., Ann. Hepatol. 2009, 8:103-112), Imiquimod (Somani, N. & Rivers, J. K., Skin Therapy Lett. 2005, 10:1-8), and AZD8848 (Hennessey 2010, Id.) TLR modulators (agonist) for TLR8 include VTX-1463 (Hennessey 2010, Id.) TLR modulators (agonist) for TLR7 and TLR8 include Resiquimod (Mark, K. E., et al., J. Infect. Dis. 2007, 195:1324-1331; Pockros, P. J., et al., J. Hepatol. 2007, 47:174-182). TLR modulators (antagonists) for TLR7 and TLR9 include IRS-954 (Barrat, F. J., et al., Eur. J. Immunol. 2007, 37:3582-3586), and IMO-3100 (Jiang, W., et al., J. Immunol. 2009. 182:48.25). TLR9 agonists include SD-101 (Barry, M. & Cooper, C., Expert Opin. Biol. Ther. 2007, 7:1731-1737), IMO-2125 (Agrawal, S. & Kandimalla. E. R., Biochem. Soc. Trans. 2007, 35:1461-1467), Bio Thrax plus CpG-7909 (Gu, M., et al., Vaccine 2007, 25:528-534), AVE0675 (Parkinson, T., Curr. Opin. Mol. Ther. 2008, 10:21-31), QAX-935 (Panter, G., et al., Curr. Opin. Mol Ther. 2009, 11:133-145), SAR-21609 (Parkinson 2008, Id.), and DIMS0150 (Pastorelli, L., et al., Expert Opin. Emerg. Drugs 2009, 14:505-521).

### TLR7 Ligands and Conjugates Thereof

With regard to TLR7 ligands and conjugates thereof, as used herein, the terms “alkyl,” “alkenyl” and “alkynyl” may include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2 propenyl, 3 butynyl, and the like. The total number of carbon atoms in each such group is sometimes described herein, e.g., when the group can contain up to ten carbon atoms it can be represented as 1-10C or as C₁-C₁₀ or C₁₋₁₀. When heteroatoms (N, O and S typically) are allowed to replace carbon atoms as in heteroalkyl groups, for example, the numbers describing the group, though still written as e.g. C₁-C₆, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain one 10C (alkyl) or two 10C (alkenyl or alkynyl). For example, they contain one 8C (alkyl) or two 8C (alkenyl or alkynyl). Sometimes they contain one 4C (alkyl) or two 4C (alkenyl or alkynyl). A single group can include more than one type of multiple bond, or more than one multiple bond: such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C₁-C₈ alkyl, C₂-C₈ heteroalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halo, ═O, ═N—CN, ═N—OR′, ═NR, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C₁-C₈ alkyl, C₂-C₈ heteroalkyl, C₁-C₈ acyl, C₂-C₈ heteroacyl, C₅-C₁₀ aryl or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group.

“Acetylene” substituents may include 2-10C alkynyl groups that are optionally substituted, and are of the formula —C≡C—Ri, wherein Ri is H or C₁-C₈ alkyl, C₂-C₈ heteroalkyl, C₂-C₈ alkenyl, C₂-C₈ heteroalkenyl, C₂-C₈ alkynyl, C₂-C₈ heteroalkynyl, C₁-C₈ acyl, C₂-C₈ heteroacyl, C₆-C₁₀ aryl, C₅-C₁₀ heteroaryl, C₇-C₁₂ arylalkyl, or C₆-C₁₂ heteroarylalkyl, and each Ri group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C₁-C₈ alkyl, C₂-C₈ heteroalkyl, C₁-C₈ acyl, C₂-C₈ heteroacyl, C₆-C₁₀ aryl, C₅-C₁₀ heteroaryl, C₇₋₁₂ arylalkyl, or C₆₋₁₂ heteroarylalkyl, each of which is optionally substituted with one or more groups selected from halo, C₁-C₄ alkyl, C₁-C₄ heteroalkyl, C₁-C₆ acyl, C₁-C₆ heteroacyl, hydroxy, amino, and ═O; and wherein two R′ can be linked to form a 3-7 membered ring optionally containing up to three heteroatoms selected from N, O and S. In some embodiments, Ri of —C≡C-Ri is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain one to three O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5 membered rings as well as 6 membered rings. Typical heteroaromatic systems include monocyclic C₅-C₆ aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. For example, the monocyclic heteroaryls may contain 5-6 ring members, and the bicyclic heteroaryls contain 8-10 ring members.

Aryl and heteroaryl moieties may be substituted with a variety of substituents including C₁-C₈ alkyl, C₂-C₈ alkenyl, C₂-C₈ alkynyl, C₅-C₁₂ aryl, C₁-C₈ acyl, and heteroforms of these, each of which can itself be further substituted: other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C₁-C₈ alkyl, C₂-C₈ heteroalkyl, C₂-C₈ alkenyl, C₂-C₈ heteroalkenyl, C₂-C₈ alkynyl, C₂-C₈ heteroalkynyl, C₆-C₁₀ aryl, C₅-C₁₀ heteroaryl, C₇-C₁₂ arylalkyl, or C₆-C₁₂ heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly. “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. For example, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group may include a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus, a benzyl group is a C₇-arylalkyl group, and phenylethyl is a C₈-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and for instance n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R² is alkyl, this alkyl may optionally be substituted by the remaining substituents listed as embodiments for R² where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

In various embodiments, the invention provides a method to prevent, inhibit or treat liver disease such as one associated with inflammation in a mammal. The methods include administering to a mammal in need thereof an effective amount of a compound of Formula (I):

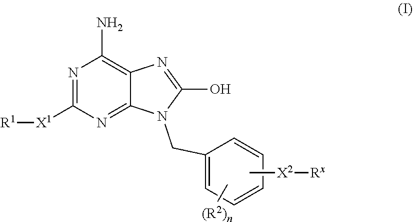

wherein X¹ is —O—, —S—, or —NR^{c}—:

R¹ is hydrogen, (C₁-C₁₀)alkyl, substituted (C₁-C₁₀)alkyl, C₆₋₁₀aryl, or substituted C₅₋₁₀aryl, C₅₋₉heterocyclic, substituted C₅₋₉heterocyclic:

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₈)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent:

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₈)alkoxy, substituted (C₁-C₆)alkoxy, (C₁-C₆)alkanoyl, substituted (C₁-C₆)alkanoyl, aryl, aryl(C₁-C₆)alkyl, Het. Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

in one embodiment, R^{x} is a phospholipid comprising one or two carboxylic esters, or comprises —(R³)ᵣ—(R⁴)ₛ)ₚ wherein each R³ independently is a polyethylene glycol (PEG) moiety; wherein each R⁴ independently is H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring. substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic: wherein r is 1 to 1000, where s is 1 to 100 and where p is 1 to 100;

or a tautomer thereof;

or a pharmaceutically acceptable salt or solvate thereof.

In one embodiment, R³ is a PEG moiety.

In some embodiments, a PEG reactant has a structure CH₃O(CH₂CH₂O)ₙ— X—NHS*, where X can be —COCH₂CH₂COO—, —COCH₂CH₂CH₂ COO—, —CH₂COO—, and —(CH₂)₅COO—. In certain embodiments, a PEG reactant has a structure

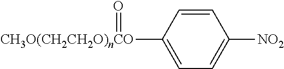

CH₃O(CH₂CH₂O)ₙ—CH₂CH₂CHO

CH₃O(CH₂CH₂O)ₙ—CH₂CH₂CH₂NH₂

CH₃O(CH₂CH₂O)ₙ—CH₂CH₂SH
or

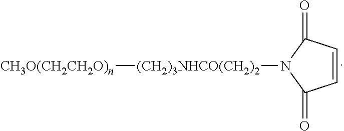

Certain PEG reactants are bifunctional in some embodiments. Examples of bifunctional PEG reactants have a structure X—(OCH₂CH₂)n-X, where X is (N-Succinimidyloxycarbonyl)methyl (—CH₂COO—NHS), Succinimidylglutarate (—COCH₂CH₂CH₂COO—NHS), (N-Succinimidyloxycarbonyl)pentyl (—(CH₂)₅COO—NHS), 3-(N-Maleimidyl)propanamido, (—NHCOCH₂CH₂-MAL), Aminopropyl (—CH₂CH₂CH₂NH₂) or 2-Sulfanylethyl (—CH₂CH₂SH) in some embodiments.

In certain embodiments, some PEG reactants are heterofunctional. Examples of heterofunctional PEG reactants have the structures

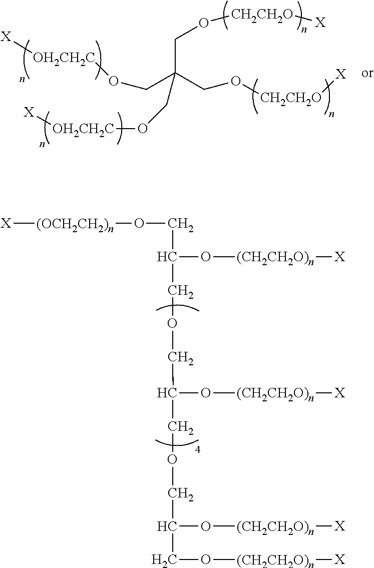

where X can be (N-Succinimidyloxycarbonyl)methyl (—CH₂COO—NHS), Succinimidylglutarate (—COCH₂CH₂CH₂COO—NHS), (N-Succinimidyloxycarbonyl)pentyl (—(CH₂)₅COO—NHS), 3-(N-Maleimidyl)propanamido, (—NHCOCH₂CH₂-MAL), 3-aminopropyl (—CH₂CH₂CH₂NH₂), 2-Sulfanylethyl (—CH₂CH₂SH), 5-(N-Succinimidyloxycarbonyl)pentyl (—(CH₂)₅COO—NHS], or p-Nitrophenyloxycarbonyl, (—CO₂-p-C₆H₄NO₂), in some embodiments.

Certain branched PEG reactants also may be utilized, such as those having a structure:

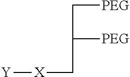

where X is a spacer and Y is a functional group, including, but not limited to, maleimide, amine, glutaryl-NHS, carbonate-NHS or carbonate-p-nitrophenol, in some embodiments. An advantage of branched chain PEG reactants is that they can yield conjugation products that have sustained release properties.

A PEG reactant also may be a heterofunctional reactant, such as

HO(CH₂CH₂O)ₙ—CH₂CH₂CH₂NH₂

HCl.H₂N—CH₂CH₂O(CH₂CH₂O)ₙ—(CH₂)₅COOH

and

HO(CH₂CH₂O)ₙ—CH₂CH₂CHO

in certain embodiments. In some embodiments, Boc*-protected-Amino-PEG-Carboxyl-NHS or Maleimide-PEG-Carboxyl-NHS reactants can be utilized.

In certain embodiments, a comb-shaped polymer may be utilized as a PEG reactant to incorporate a number of PEG units into a conjugate. An example of a comb-shaped polymer is shown hereafter.

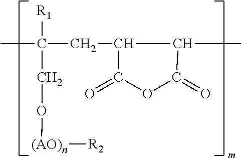

A PEG reactant, and/or a PEG conjugate product, can in some embodiments have a molecular weight ranging between about 5 grams per mole to about 100,000 grams per mole. In some embodiments, a PEG reactant, and/or a PEG conjugate product, has a average, mean or nominal molecular weight of about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000 or 90000 grams per mole. In some embodiments the PEG moiety in a compound herein is homogeneous and the molecule weight of the PEG moiety is the same for each molecule of a particular batch of compound (e.g., R³ is one PEG unit and r is 2 to 10).

In various embodiments, X2 in formula (I) can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

Certain non-limiting examples of X² in formula (I) include —(Y)_{y}, —(Y)_{y}—C(O)N—(Z)_{z}—, —(CH₂)_{y}—C(O)N—(CH₂)_{z}—, —(Y)_{y}—NC(O)—(Z)_{z}—, —(CH₂)_{y}—NC(O)—(CH₂)_{z}—, where each y (subscript) and z (subscript) independently is 0 to 20 and each Y and Z independently is C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C₁₋₆ alkoxy C₁₋₆ alkenyl, amino, cyano, halogen or aryl. In certain embodiments, a linker sometimes is a —C(Y′)(Z′)—C(Y″)(Z″)— linker, where each Y′, Y″, Z′ and Z″ independently is hydrogen C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Hat C₁-C₆ alkyl, or C₁—C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₅-C₉ cycloalkyl, C₅-C₉ heterocyclic, C1-6 alkoxy C₁₋₆ alkenyl, amino, cyano, halogen or aryl.

Another specific value for X² in formula (I) is

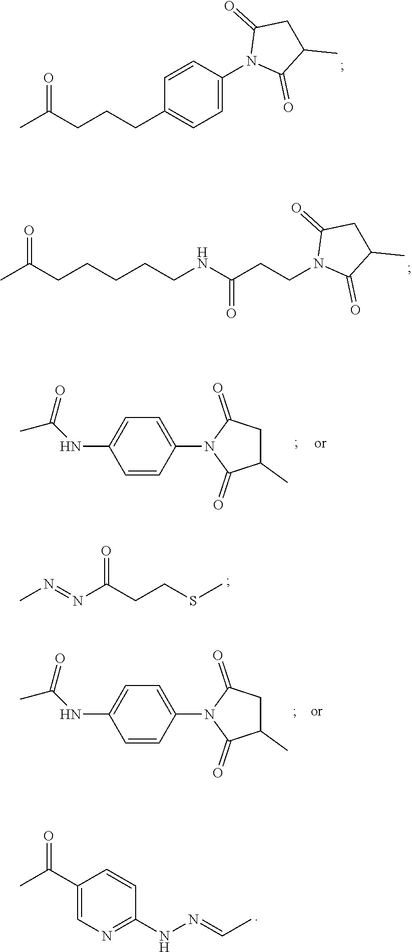

Another specific value for X² is

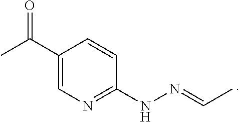

In various embodiments, X2 can be C(O), or can be an of

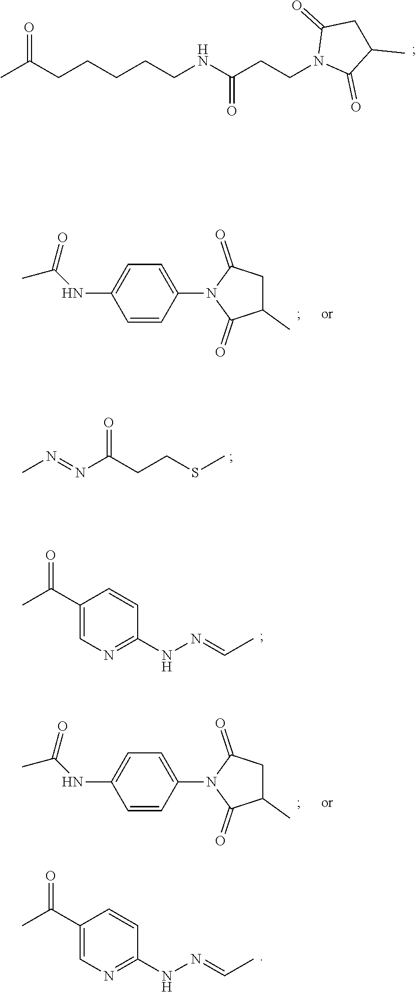

In various embodiments, X¹ in formula (I) can be oxygen.

In various embodiments, X¹ in formula (I) can be sulfur, or can be —NR^{c}— where R^{c} is hydrogen, C₁₋₆ alkyl or substituted C₁₋₆ alkyl, where the alkyl substituents are hydroxy, C₃₋₆cycloalkyl, C₁₋₆alkoxy, amino, cyano, or aryl. More specifically, X¹ can be —NH—.

In various embodiments, R¹ and R in formula (I) taken together can form a heterocyclic ring or a substituted heterocyclic ring. More specifically, R¹ and R^{c} taken together can form a substituted or unsubstituted morpholino, piperidino, pyrrolidino, or piperazino ring.

In various embodiments R¹ in formula (I) can be a C₁-C₁₀ alkyl substituted with C₁₋₆ alkoxy.

In various embodiments, R¹ in formula (I) can be hydrogen, C₁₋₄alkyl, or substituted C₁₋₄alkyl. More specifically, R¹ can be hydrogen, methyl, ethyl, propyl, butyl, hydroxyC₁₋₄alkylene, or C₁₋₄alkoxyC₁₋₄alkylene. Even more specifically, R¹ can be hydrogen, methyl, ethyl, methoxyethyl, or ethoxyethyl.

In various embodiments, R² in formula (I) can be absent, or R² can be halogen or C₁₋₄alkyl. More specifically, R² can be chloro, bromo, methyl, or ethyl.

In one embodiment, R^{x} in formula (I) is ((R³)ᵣ—(R⁴)ₛ)ₚ or is R³. In one embodiment, R³ is a PEG moiety or a derivative of a PEG moiety. In certain embodiment R³ is —O—CH₂—CH₂— or —CH₂CH₂—O—. In one embodiment, a PEG moiety can include one or more PEG units. A PEG moiety can include about 1 to about 1,000 PEG units, including, without limitation, about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800 or 900 units, in some embodiments. In certain embodiments, a PEG moiety can contain about 1 to 5 up to about 25 PEG units, about 1 to 5 up to about 10 PEG units, about 10 up to about 50 PEG units, about 18 up to about 50 PEG units, about 47 up to about 150 PEG units, about 114 up to about 350 PEG units, about 271 up to about 550 PEG units, about 472 up to about 950 PEG units, about 50 up to about 150 PEG units, about 120 up to about 350 PEG units, about 250 up to about 550 PEG units or about 650 up to about 950 PEG units. A PEG unit is —O—CH₂—CH₂— or —CH₂—CH₂—O— in certain embodiments. In some embodiments, R⁴ is H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic.

In some embodiments, r is about 5 to about 100, and sometimes r is about 5 to about 50 or about 5 to about 25. In certain embodiments, r is about 5 to about 15 and sometimes r is about 10. In some embodiments, R³ is a PEG unit (PEG), and r is about 2 to about 10 (e.g., r is about 2 to about 4) or about 18 to about 500.

In some embodiments, s is about 5 to about 100, and sometimes s is about 5 to about 50 or about 5 to about 25. In certain embodiments, s is about 5 to about 15 and sometimes s is about 10. In some embodiments. s is about 5 or less (e.g., s is 1). In some embodiments, the (R³)ᵣ substituent is linear, and in certain embodiments, the (R³)ᵣ substituent is branched. For linear moieties, s sometimes is less than r (e.g., when R₃ is —O—CH₂—CH₂— or —CH₂—CH₂—O—) and at times s is 1. In some embodiments R₃ is a linear PEG moiety (e.g., having about 1 to about 1000 PEG units), s is 1 and r is 1. For branched moieties, s sometimes is less than, greater than or equal to r (e.g., when R₃ is —O—CH₂—CH₂— or —CH₂—CH₂—O—), and at times r is 1, s is 1 and p is about 1 to about 1000 (e.g., p is about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800, 900 or 1000).

In some embodiments R³ is —O—CH₂—CH₂— or —CH₂—CH₂—O— and r is about 1 to about 1000 (e.g., about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800, 900 or 1000).

In certain embodiments, X² is an amido linking group (e.g., —C(O)NH— or —NH(O)C—); alkyl amido linking group (e.g., —C₁-C₆ alkyl-C(O)NH—, —C₁-C₆ alkyl-NH(O)C—, —C(O)NH—C₁-C₆ alkyl-, —NH(O)C—C₁-C₆ alkyl-, —C₁-C₆ alkyl-NH(O)C—C₁-C₆ alkyl-, —C₁-C₆ alkyl-C(O)NH—C₁-C₆ alkyl-, or —C(O)NH—(CH₂)ₜ—, where t is 1, 2, 3, or 4); substituted 5-6 membered ring (e.g., aryl ring, heteroaryl ring (e.g., tetrazole, pyridyl, 2,5-pyrrolidinedione (e.g., 2,5-pyrrolidinedione substituted with a substituted phenyl moiety)), carbocyclic ring, or heterocyclic ring) or oxygen-containing moiety (e.g., —O—, —C₁-C₆ alkoxy).

A “phospholipid” as the term is used herein refers to a glycerol mono- or diester bearing a phosphate group bonded to a glycerol hydroxyl group with an alkanolamine group being bonded as an ester to the phosphate group, of the general formula

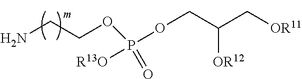

wherein R¹¹ and R¹² are each independently hydrogen or an acyl group, and R¹³ is a negative charge or a hydrogen, depending upon pH. When R13 is a negative charge, a suitable counterion, such as a sodium ion, can be present. For example, the alkanolamine moiety can be an ethanolamine moiety, such that m=1. It is also understood that the NH group can be protonated and positively charged, or unprotonated and neutral, depending upon pH. For example, the phospholipid can exist as a zwitterion with a negatively charged phosphate oxy anion and a positively charged protonated nitrogen atom. The carbon atom bearing OR¹² is a chiral carbon atom, so the molecule can exist as an R isomer, an S isomer, or any mixture thereof. When there are equal amounts of R and S isomers in a sample of the compound of formula (II), the sample is referred to as a “racemate.” For example, in the commercially available product 1,2-dioleoyl-sn-glycero-3-phosphoethanolamine, the R³ group is of the chiral structure

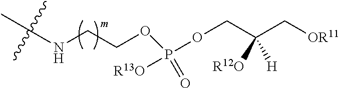

which is of the R absolute configuration.

A phospholipid can be either a free molecule, or covalently linked to another group for example as shown

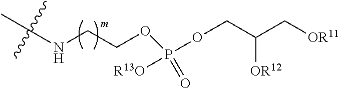

wherein a wavy line indicates a point of bonding.

Accordingly, when a substituent group, such as R^{x} of the compound of formula (I) herein, is stated to be a phospholipid what is meant that a phospholipid group is bonded as specified by the structure to another group, such as to an N-benzyl heterocyclic ring system as disclosed herein. The point of attachment of the phospholipid group can be at any chemically feasible position unless specified otherwise, such as by a structural depiction. For example, in the phospholipid structure shown above, the point of attachment to another chemical moiety can be via the ethanolamine nitrogen atom, for example as an amide group by bonding to a carbonyl group of the other chemical moiety, for example

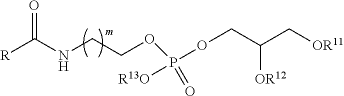

wherein R represents the other chemical moiety to which the phospholipid is bonded. In this bonded, amide derivative, the R¹³ group can be a proton or can be a negative charge associated with a counterion, such as a sodium ion. The acylated nitrogen atom of the alkanolamine group is no longer a basic amine, but a neutral amide, and as such is not protonated at physiological pH.

An “acyl” group as the term is used herein refers to an organic structure bearing a carbonyl group through which the structure is bonded, e.g., to glycerol hydroxyl groups of a phospholipid, forming a “carboxylic ester” group. Examples of acyl groups include fatty acid groups such as oleoyl groups, that thus form fatty (e.g., oleoyl) esters with the glycerol hydroxyl groups. Accordingly, when R¹¹ or R¹², but not both, are acyl groups, the phospholipid shown above is a mono-carboxylic ester, and when both R¹¹ and R¹² are acyl groups, the phospholipid shown above is a di-carboxylic ester.

In one embodiment, the phospholipid of R^{x} comprises two carboxylic esters and each carboxylic ester includes one, two, three or four sites of unsaturation, epoxidation, hydroxylation, or a combination thereof.

In one embodiment, the phospholipid of R^{x} comprises two carboxylic esters and the carboxylic esters of are the same or different.

In one embodiment, each carboxylic ester of the phospholipid is a C17 carboxylic ester with a site of unsaturation at C8-C9.

In one embodiment, each carboxylic ester of the phospholipid is a C18 carboxylic ester with a site of unsaturation at C9-C10.

In one embodiment, X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.

In one embodiment, X² is C(O),

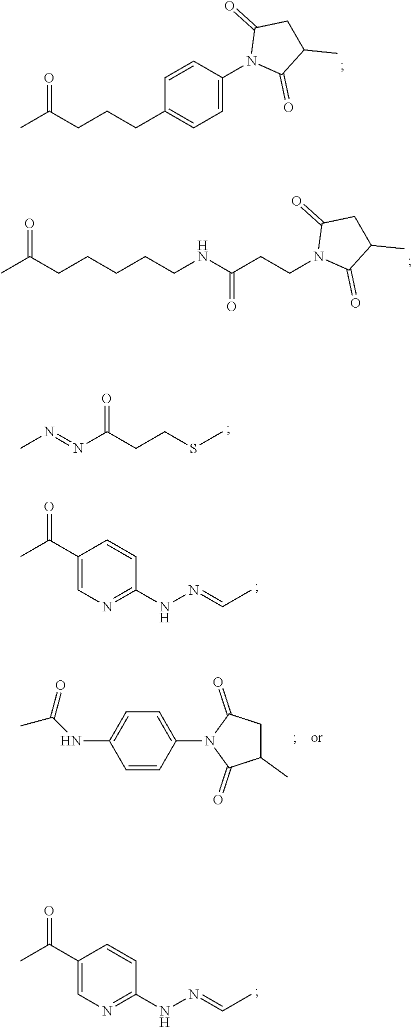

In one embodiment, R^{x} comprises dioleoylphosphatidyl ethanolamine (DOPE).

In one embodiment, R^{x} is 1,2-dioleoyl-sn-glycero-3-phospho ethanolamine and X2 is C(O).

In one embodiment, X¹ is oxygen or is —NH—.

In one embodiment, R¹ and R^{c} taken together form a heterocyclic ring or a substituted heterocyclic ring, e.g., form a substituted or unsubstituted morpholino, piperidino, pyrrolidino, or piperazino ring.

In one embodiment, R¹ is a C1-C10 alkyl substituted with C1-6 alkoxy, R¹ is hydrogen, C₁₋₄alkyl, or substituted C₁₋₄alkyl, R¹ is hydrogen, methyl, ethyl, propyl, butyl, hydroxyC₁₋₄alkylene, or C₁₋₄alkoxyC₁₋₄alkylene, or R¹ is hydrogen, methyl, ethyl, methoxyethyl, or ethoxyethyl.

In one embodiment, the composition further comprises an amount of an antigen.

In various embodiments, the mammal can be a human.

In various embodiments, the composition can be intranasally administered, or can be dermally administered, or can be systemically administered.

### TLR4 Ligands

As used herein with regard to TLR4 ligands, the term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. In one embodiment those groups have 10 or fewer carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N. P, Si, and S, and wherein the nitrogen and sulfur atoms may optionally be oxidized, and the nitrogen heteroatom may optionally be quaternized. The heteroatom(s) O, N. P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples ofheterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene.” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom.

Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g., from 1 to 3 rings) that are fused together (i.e., a fused ring aryl) or linked covalently. A fused ring aryl refers to 15 multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =0, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″)═NR″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ in one embodiment each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)z, fluoro(C₁-C₄)alkoxy, and fluoro(C₁-C₄)alkyl, in a number ranging from zero to the total number of open valences on the aromatic ring system; and where R′, R″, R′″, and R″″ are in one embodiment independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R′″)—, where sand dare independently integers of from 0 to 3, and X is —O—, —NR′—, —S—, —S(O)—, —S(O)₂—, or —S(O)₂NR′—. The substituents R, R′, R″, and R′″ are in one embodiment independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:

(A) —OH, —NH₂, —SH, —CN, —CF₃, —CCl₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or“lower substituent group,” as used herein, means a group selected from all of the substituents described above for a “substituent group,” wherein each substituted or unsubstituted alkyl is, for example, a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In some embodiments, a compound as described herein may include multiple instances of a substituent, e.g., R⁵, R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C}. In such embodiments, each substituent may optional be different at each occurrence and be appropriately labeled to distinguish each group for greater clarity. For example, where each R^{5A} is different, they may be referred to as e.g., R^{5A.1}, R^{5A.2}, R^{5A.3}, R^{5A.4}, R^{5A.5}. Similarly, where any of R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C} multiply occur, the definition of each occurrence of R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C} assumes the definition of R^{5A}, R^{5B}, R^{5C}, R^{6A}, R^{6B}, R^{6C}, R⁷, R^{7A}, R^{7B}, R^{7C}, R⁸, R^{8A}, R^{8B} and/or R^{8C}, respectively.

In one aspect, there is provided a compound having formula (II):

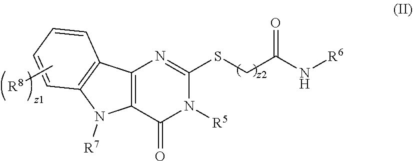

or a pharmaceutically acceptable salt thereof. In formula (II), zI is an integer from 0 to 4, and z2 is an integer from 0 to 5. R⁵ is substituted or unsubstituted cycloalkyl, substituted or unsubstituted heterocycloalkyl, substituted or unsubstituted aryl, or substituted or unsubstituted heteroaryl, R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, R⁷ is hydrogen, or substituted or unsubstituted alkyl, and R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In one embodiment, R⁵ is R^{5A}-substituted or unsubstituted cycloalkyl, R^{5A} substituted or unsubstituted heterocycloalkyl, R^{5A} substituted or unsubstituted aryl, or R^{5A} substituted or unsubstituted heteroaryl. R^{5A} is independently halogen, —CN, —CF₃, —CCl₃, —OH, —NH₂, —SO₂, —COOH, oxo, nitro, —SH, —CONH₂, R^{5B}-substituted or unsubstituted alkyl, R^{5B}-substituted or unsubstituted heteroalkyl, R^{5B}-substituted or unsubstituted cycloalkyl, R^{5B}-substituted or unsubstituted heterocycloalkyl, R^{5B}-substituted or unsubstituted aryl, or R^{5B}-substituted or unsubstituted heteroaryl. R^{5B} is independently halogen, —CN, —CF₃, —CCl₃, —OH, —NH₂, —SO₂, —COOH, oxo, nitro, —SH, —CONH₂, R^{5C}-substituted or unsubstituted alkyl, R^{5C}-substituted or unsubstituted heteroalkyl, R^{5C}-substituted or unsubstituted cycloalkyl. R^{5C}-substituted or unsubstituted heterocycloalkyl, R^{5C}-substituted or unsubstituted aryl, or RSC-substituted or unsubstituted heteroaryl. R^{5C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R⁶ is R^{6A}-substituted or unsubstituted alkyl, R^{6A} substituted or unsubstituted heteroalkyl, R^{6A} substituted or unsubstituted cycloalkyl, R^{6A} substituted or unsubstituted heterocycloalkyl, R^{6A} substituted or unsubstituted aryl, or R^{6A} substituted or unsubstituted heteroaryl. R^{6A} is independently halogen, —CN, —CF₃, —CCl₃, —OH, —NH₂, —SO₂, —COOH, oxo, nitro, —SH, —CONH₂, R^{6B}-substituted or unsubstituted alkyl, R^{6B}-substituted or unsubstituted heteroalkyl, R^{6B}-substituted or unsubstituted cycloalkyl, R^{6B}-substituted or unsubstituted heterocycloalkyl, R^{6B}-substituted or unsubstituted aryl, or 10 R^{6B}-substituted or unsubstituted heteroaryl. R^{6B} is independently halogen, —CN, —CF₃, —CCl₃, —OH, —NH₂, —SO₂, —COOH, oxo, nitro, —SH, —CONH₂, R^{6C}-substituted or unsubstituted alkyl, R^{6C}-substituted or unsubstituted heteroalkyl, R^{6C}-substituted or unsubstituted cycloalkyl, R^{6C}-substituted or unsubstituted heterocycloalkyl, R^{6C}-substituted or unsubstituted aryl, or R^{6C}-substituted or unsubstituted heteroaryl. R^{6C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R⁷ is hydrogen, or R^{7A}-substituted or unsubstituted alkyl. R^{7A} is independently halogen, —CN, —CF₃, —CC, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R⁸ is independently halogen, —CN, —SH, —OH, —COOH, —NH₂, —CONH₂, nitro, —CF₃, —CCl₃, R^{8A}-substituted or unsubstituted alkyl, R^{8A}-substituted or unsubstituted heteroalkyl, R^{8A} substituted or unsubstituted cycloalkyl, R^{8A}-substituted or unsubstituted heterocycloalkyl, R^{8A} substituted or unsubstituted aryl, or R^{8A}-substituted or unsubstituted heteroaryl. R^{8A} is independently halogen, —CN, —CF₃, —CCl₃, —OH, —NH₂, —SO₂, —COOH, oxo, nitro, —SH, —CONH₂, R^{8B}-substituted or unsubstituted alkyl, R^{8B}-substituted or unsubstituted heteroalkyl, R^{8B}-substituted or unsubstituted cycloalkyl, R^{8B}-substituted or unsubstituted heterocycloalkyl, R^{8B}-substituted or unsubstituted aryl, or R^{8B}-substituted or unsubstituted heteroaryl. R^{8B} is independently halogen, —CN, —CF₃, —CCl₃, —OH, —NH₂, —SO₂, —COOH, oxo, nitro, —SH, —CONH₂, R^{8C}-substituted or unsubstituted alkyl, R^{8C}-substituted or unsubstituted heteroalkyl, R^{8C}-substituted or unsubstituted cycloalkyl, R^{8C}-substituted or unsubstituted heterocycloalkyl, R^{8C}-substituted or unsubstituted aryl, or R^{8C}-substituted or unsubstituted heteroaryl. R^{8C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In another aspect, there is provided a compound of formula (II) as disclosed above, provided, however, that: (i) the compound of formula (II) is not

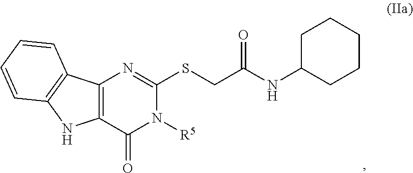

wherein R⁵ is p-fluorophenyl or p-methylphenyl; (ii) the compound is not

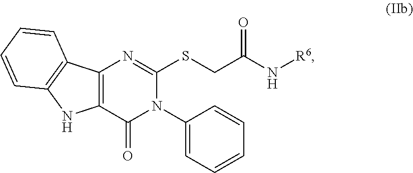

wherein R⁶ is unsubstituted aryl, unsubstituted cyclohexyl, unsubstituted thiazole, or —CH₂-furanyl; or (iii) R⁷ is not hydrogen.

Further to any aspect disclosed above, in one embodiment, R⁵ is not substituted phenyl. In one embodiment, R⁵ is not p-fluorophenyl or p-methylphenyl.

In one embodiment, the compound does not have the structure of formula (IIa) wherein R⁶ is substituted phenyl. In one embodiment, the compound does not have the structure of formula (IIa) wherein R⁶ is p-fluorophenyl or p-methylphenyl.

Further to any aspect disclosed above, in one embodiment, R⁶ is not substituted or unsubstituted aryl, unsubstituted cyclohexyl, unsubstituted thiazole, or —CH₂-furanyl.

In one embodiment, the compound does not have the structure of formula (IIb) wherein R⁶ is substituted or unsubstituted aryl, substituted or unsubstituted cyclohexyl, substituted or unsubstituted thiazole, or alkyl substituted with a substituted or unsubstituted furanyl. In one embodiment, R⁶ is not unsubstituted aryl, unsubstituted cyclohexyl, unsubstituted thiazole, or —CH₂-furanyl.

Further to any aspect disclosed above, in one embodiment R⁶ is substituted or unsubstituted cycloalkyl or substituted or unsubstituted aryl. In one embodiment, R⁶ is unsubstituted cycloalkyl or unsubstituted aryl.

In one embodiment, R⁶ is substituted or unsubstituted C₆-C₈ cycloalkyl or substituted or unsubstituted phenyl. In one embodiment, R⁶ is substituted or unsubstituted Ce, cycloalkyl or substituted or unsubstituted phenyl.

In one embodiment, R⁵ is R^{5A}-substituted or unsubstituted C6 cycloalkyl or R^{5A}-substituted or unsubstituted phenyl, wherein R^{5A} is a halogen. In one embodiment, R⁵ is R^{5A}-substituted or unsubstituted phenyl, wherein R^{5A} is a halogen. In one embodiment, R⁵ is R^{5A}-substituted or unsubstituted phenyl, wherein R^{5A} is a fluoro. In one embodiment, R⁵ is unsubstituted phenyl.

Further to any aspect disclosed above, in one embodiment the compound does not have the structure of Formula (Ib) wherein R⁶ is substituted or unsubstituted aryl, substituted or unsubstituted cyclohexyl, substituted or unsubstituted thiazole, or alkyl substituted with a substituted or unsubstituted furanyl.

In one embodiment, R⁶ is substituted or unsubstituted C₄-C₁₂ cycloalkyl, substituted or unsubstituted C₃-C₁₂ alkyl, substituted or unsubstituted aryl, or substituted or unsubstituted heteroaryl. In one embodiment, R⁶ is substituted or unsubstituted C₄-C₁₂ cycloalkyl, substituted or unsubstituted C₄-C₁₂ alkyl, substituted or unsubstituted aryl, or substituted or unsubstituted heteroaryl. In one embodiment, R⁶ is substituted or unsubstituted C₄-C₁₂ cycloalkyl, substituted or unsubstituted C₄-C₁₂ branched alkyl, or substituted or unsubstituted phenyl. In one embodiment, R⁶ is R^{6A}-substituted or unsubstituted C₄-C₁₂ cycloalkyl, R^{6A}-substituted or unsubstituted C₄-C₁₂ branched alkyl, or R^{6A}-substituted or unsubstituted phenyl, wherein R^{6A} is halogen. In one embodiment, R⁶ is R^{6A}-substituted or unsubstituted C₄-C₁₂ cycloalkyl, R^{6A}-substituted or unsubstituted C₄-C₁₂ branched alkyl, or R^{6A}-substituted or unsubstituted phenyl, wherein R^{6A} is fluoro. In one embodiment, R⁶ is unsubstituted C₄-C₁₂ cycloalkyl, unsubstituted C₄-C₁₂ branched alkyl, or R^{6A}-substituted or unsubstituted phenyl, wherein R^{6A} is fluoro. In one embodiment, R⁶ is unsubstituted C₆-C₁₂ cycloalkyl, unsubstituted C₄-C₁₂ branched alkyl, or unsubstituted phenyl. In one embodiment, R⁶ is unsubstituted C₆-C₁₀ cycloalkyl. In one embodiment, R⁶ is unsubstituted C₆-C₈ cycloalkyl. In one embodiment, R⁶ is unsubstituted cyclohexyl.

In one embodiment, R⁷ is hydrogen or substituted or unsubstituted alkyl. In one 30 embodiment, R⁷ is hydrogen or unsubstituted alkyl. In one embodiment, R⁷ is hydrogen or unsubstituted C₁-C₃ alkyl. In one embodiment, R⁷ is hydrogen, methyl or ethyl. In one embodiment, R³ is methyl. In one embodiment, R⁷ is ethyl. In one embodiment, R⁷ is hydrogen.

In one embodiment, zI is 0, 1, 2, 3, or 4. In one embodiment, zI is 0 or 1. In one embodiment, zI is 0. In one embodiment, zI is 1. In one embodiment, z2 is 0, 1, 2, 3, 4, or 5. In one embodiment, z2 is 1.

In one embodiment, R⁸ is independently substituted or unsubstituted alkyl. In one embodiment, R⁸ independently is substituted alkyl. In one embodiment, R⁸ is independently unsubstituted alkyl. In one embodiment. R⁸ is independently substituted or unsubstituted heteroalkyl. In one embodiment, R⁸ is independently substituted heteroalkyl. In one embodiment, R⁸ is independently unsubstituted heteroalkyl. In one embodiment, R⁸ is independently substituted or unsubstituted aryl. In one embodiment, R⁸ is independently substituted or unsubstituted heteroaryl.

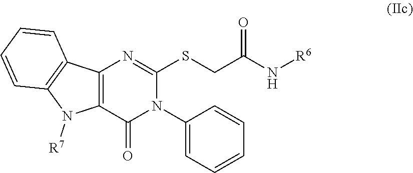

For formula (IIc) (above), R⁶ is substituted or unsubstituted alkyl, or substituted or unsubstituted cycloalkyl; and R⁷ is substituted or unsubstituted alkyl. In one embodiment, R⁶ is unsubstituted cycloalkyl, e.g., cyclohexyl, cycloheptyl or cyclooctyl. In one embodiment, R⁶ is unsubstituted alkyl, e.g., 3,3-dimethylbutyl. In one embodiment, R⁷ is unsubstituted alkyl. In one embodiment, R¹⁰ is an alkyl ester.

In another aspect, there is provided a compound having formula (IId):

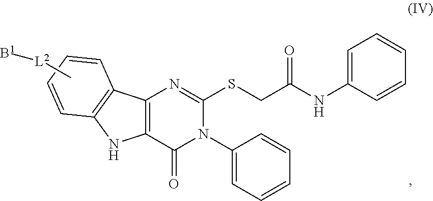

For formula (IId), L² is a linker, and B¹ is a purine base or analog thereof.

In one embodiment, L² is a substituted or unsubstituted alkylene, or a substituted or unsubstituted heteroalkylene. In one embodiment, L² includes a water soluble polymer. A “water soluble polymer” means a polymer which is sufficiently soluble in water under physiologic conditions of e.g., temperature, ionic concentration and the like, as known in the art, to be useful for the methods described herein. An exemplary water soluble polymer is polyethylene glycol.

In one embodiment, the water soluble polymer is -(0C₂CH₂)ₘ— wherein m is 1 to 100. In one embodiment, L² includes a cleavage element. A “cleavage element” is a chemical functionality which can undergo cleavage (e.g., hydrolysis) to release the compound, optionally including remnants of linker L², and B¹, optionally including remnants 20 of linker L².

TABLE 1

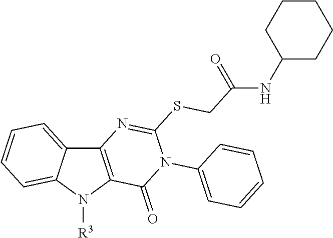

| | | mouse | | | human | |
|---|---|---|---|---|---|---|
| Compound | R³ | IL-6^{a} | IP-10^{b} | TLR4^{a} | IL-8^{c} | TLR4^{c} |
| 1 | H | 100 | 100 | 100 | 100 | 100 |
| 42 | CH₃ | 101 | 99 | 100 | 79 | 92 |
| 43 | CH₃, N-methyl | <1 | <1 | 1 | 5 | <1 |
| 44 | 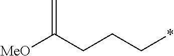 [SMILES: *CCCC(=C)OC] | 4 | 19 | 9 | 124 | 19 |
| 48 | 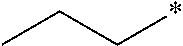 [SMILES: *CCC] | 49 | 45 | 43 | 83 | 117 |
| 49 | 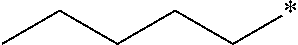 [SMILES: *CCCCC] | 5 | <1 | 6 | 1 | 11 |
| 50 | 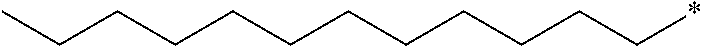 [SMILES: *CCCCCCCCCCCC] | <1 | <1 | 4 | <1 | 10 |
| 51 | 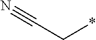 [SMILES: *CC#N] | 98 | 64 | 55 | 30 | 19 |
| 52 | 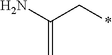 [SMILES: *CC(=C)N] | 1 | 1 | 8 | 4 | 11 |

TABLE 2

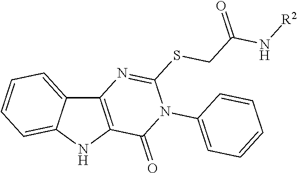

| | | mouse | | | human | |
|---|---|---|---|---|---|---|
| Compound | R² | IL-6^{a} | IP-10^{b} | TLR4^{c} | IL-8^{d} | TLR4^{e} |
| 1 | 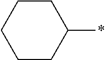 [SMILES: *C1CCCCC1] | 100 | 100 | 100 | 100 | 100 |
| 9 | 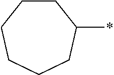 [SMILES: *C1CCCCCC1] | 126 | 107 | 99 | 118 | 100 |
| 10 | 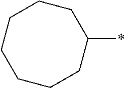 [SMILES: *C1CCCCCCC1] | 114 | 98 | 95 | 73 | 64 |
| 11 | 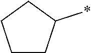 [SMILES: *C1CCCC1] | 53 | 52 | 28 | 5 | 23 |
| 12 | 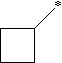 [SMILES: *C1CCC1] | 35 | 66 | 18 | 19 | 32 |
| 13 | 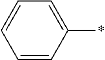 [SMILES: *c1ccccc1] | 11 | 44 | 21 | 15 | 31 |
| 14 | 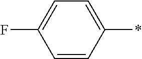 [SMILES: *c1ccc(F)cc1] | 103 | 70 | 81 | 107 | 108 |
| 15 | 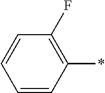 [SMILES: *c1ccccc1F] | 36 | 54 | 43 | 61 | 71 |
| 16 | 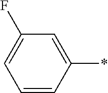 [SMILES: *c1cccc(F)c1] | 14 | 45 | 11 | 13 | 39 |
| 17 | 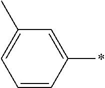 [SMILES: *c1cccc(C)c1] | 19 | 55 | 13 | 54 | 116 |
| 18 | 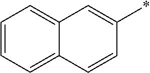 [SMILES: *c1ccc2ccccc2c1] | <1 | <1 | 2 | <1 | 21 |
| 19 | 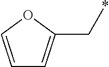 [SMILES: *Cc1ccco1] | 18 | 26 | 5 | 15 | 27 |
| 20 | 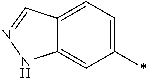 [SMILES: *c1ccc2cn[nH]c2c1] | 3 | <1 | <1 | 7 | <1 |
| 21 | 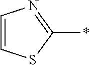 [SMILES: *c1nccs1] | 1 | <1 | 2 | 5 | 12 |
| 22 | 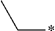 [SMILES: *CC] | <1 | <1 | <1 | 4 | 8 |
| 23 | 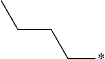 [SMILES: *CCCC] | 49 | 61 | 31 | 29 | 32 |
| 24 | 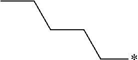 [SMILES: *CCCCC] | 40 | 40 | 42 | 25 | 23 |
| 25 | 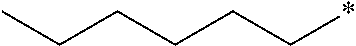 [SMILES: *CCCCCC] | <1 | <1 | 4 | 7 | 8 |
| 26 | 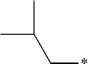 [SMILES: *CC(C)C] | 44 | 53 | 24 | 11 | 21 |
| 27 | 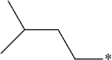 [SMILES: *CCC(C)C] | 61 | 62 | 56 | 53 | 38 |
| 28 | 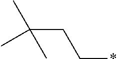 [SMILES: *CCC(C)(C)C] | 99 | 85 | 126 | 95 | 72 |
| 29 | 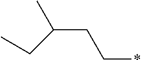 [SMILES: *CCC(C)CC] | 30 | 45 | 29 | 23 | 17 |
| 30 | 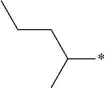 [SMILES: *C(C)CCC] | 12 | 55 | 17 | 6 | 13 |
| 31 | 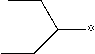 [SMILES: *C(CC)CC] | 10 | 26 | 8 | <1 | 11 |
| 32 | 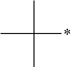 [SMILES: *C(C)(C)C] | <1 | <1 | <1 | 5 | 6 |
| 33 | 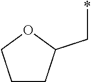 [SMILES: *CC1CCCO1] | 23 | 54 | 21 | 8 | 13 |
| 34 35 | 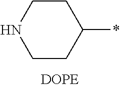 [SMILES: *C1CCNCC1] | <1 <1 | <1 <1 | <1 <1 | 17 <1 | <1 <1 |

TABLE 3

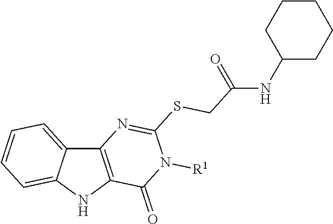

| Com- | | mouse | | | human | |
|---|---|---|---|---|---|---|
| pound | R¹ | IL-6^{a} | IP-10^{b} | TLR4^{c} | IL-8^{d} | TLR4^{e} |
| 1 | 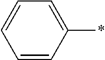 [SMILES: *c1ccccc1] | 100 | 100 | 100 | 100 | 100 |
| 36 | 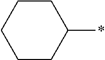 [SMILES: *C1CCCCC1] | 71 | 61 | 56 | 27 | 40 |
| 37 | 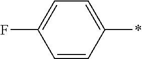 [SMILES: *c1ccc(F)cc1] | 48 | 72 | 41 | 5 | 51 |
| 38 | 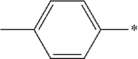 [SMILES: *c1ccc(C)cc1] | <1 | <1 | 2 | 6 | 2 |
| 39 | 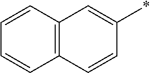 [SMILES: *c1ccc2ccccc2c1] | 3 | <1 | 6 | <1 | 3 |
| 40 | 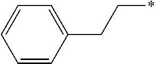 [SMILES: *CCc1ccccc1] | <1 | <1 | 1 | 5 | 1 |
| 41 | 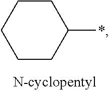 | 47 | 47 | 19 | 14 | 33 |

### Routes and Formulations

Administration of compositions having one or more antigens and one or more adjuvants and optionally another active agent or administration of a composition having one or more antigens and a composition having one or more adjuvants, can be via any of suitable route of administration, particularly parenterally, for example, intravenously, intra-arterially, intraperitoneally, intrathecally, intraventricularly, intraurethrally, intrasternally, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

The compositions invention alone or in combination with other active agents can be formulated as pharmaceutical compositions and administered to a mammalian host, such as a human patient in a variety of forms adapted to the chosen route of administration. e.g., orally or parenterally, by intravenous, intramuscular, topical or subcutaneous routes.

Thus, the compositions alone or in combination with another active agent, e.g., an antigen, may be systemically administered. e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patients diet. For oral therapeutic administration, the composition optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The composition optionally in combination with another active compound may also be administered intravenously or intraperitoneally by infusion or injection. Solutions of the antigen(s), and adjuvant(s) optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the antigen(s) and adjuvant(s) optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to enhance the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the invention provides various dosage formulations of the antigen(s) and adjuvant(s) optionally in combination with another active compound for inhalation delivery. For example, formulations may be designed for aerosol use in devices such as metered-dose inhalers, dry powder inhalers and nebulizers.

Examples of useful dermatological compositions which can be used to deliver compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages can be determined by comparing their in vitro activity, and in vivo activity in animal models. Methods for the extrapolation of effective dosages in mice, and other animals, to humans are known to the art; for example, see U.S. Pat. No. 4,938,949. The ability of an adjuvant to act as a TLR agonist may be determined using pharmacological models which are well known to the art, including the procedures disclosed by Lee et al., Proc. Natl. Acad. Sci. USA, 100: 8646 (2003).

Generally, the concentration of the phospholipid optionally in combination with another active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the antigen(s) and adjuvant(s) optionally in combination with another active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The antigen(s) and adjuvant(s) optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 uM, which can be effective to kill cancer cells. In managing the patient, the therapy should be initiated at a lower dose and incd depending on the patient's global response.

A specific antigen includes an amino acid, a carbohydrate, a peptide, a protein, a nucleic acid, a lipid, a body substance, or a cell such as a microbe.

A specific peptide has from 2 to about 20 amino acid residues.

Another specific peptide has from 10 to about 20 amino acid residues.

A specific antigen includes a carbohydrate.

A specific antigen is a microbe. A specific microbe is a virus, bacteria, or fungi.

Specific bacteria are Bacillus anthracis, Listeria monocytogenes, Francisella tularensis, Salmonella, or Staphylococcus. Specific Salmonella are S. typhimurium or S. enteritidis. Specific Staphylococcus include S. aureus.

Specific viruses are RNA viruses, including RSV and influenza virus, a product of the RNA virus, or a DNA virus, including herpes virus. A specific DNA virus is hepatitis B virus.

The invention includes compositions that include of a TLR4 agonist and TLR7 agonist phospholipid conjugate optionally in combination with other active agents that may or may not be antigens, e.g., ribavirin, mizoribine, and mycophenolate mofetil.

### EXEMPLARY EMBODIMENTS

In one embodiment, a method to enhance an immune response in a mammal is

provided. In one
embodiment, the method comprises administering to a mammal in need thereof a composition comprising an
effective amount of a TLR4 agonist and a TLR7 agonist. In one embodiment, the composition is a liposomal
composition. In one embodiment, the composition comprises liposomes comprising a TLR4 agonist and
liposomes comprising a TLR7 agonist. In one embodiment, the composition comprises liposomes comprising
a TLR4 agonist and a TLR7 agonist. In one embodiment, the TLR4 agonist and a TLR7 agonist are
administered simultaneously. In one embodiment, the TLR4 agonist has formula (II). In one
embodiment, the TLR4 agonist comprises 1Z105, 2B182c, INI-2004, or CRX601. In one embodiment, the
TRL4 agonist is not 1Z105. In one embodiment, the TLR7 agonist has formula (I). In one embodiment, the
liposomes comprise PC, DOPC, or DSPC. In one embodiment, the liposomes comprise cholesterol. In one
embodiment, the method further comprises administering one or more immunogens. In one embodiment, the
immunogen is a microbial immunogen, e.g., one or more microbial proteins, glycoproteins, saccharides and/or
lipopolysaccharides. In one embodiment, the microbe is a virus, such as influenza or varicella, or a bacteria.
In one embodiment, the microbe is a parasite or fungus. In one embodiment, the liposomes comprise the one
or more immunogens. In one embodiment, the composition comprises the one or more immunogens. In one
embodiment, the mammal is a human. In one embodiment, the mammal is a rodent, equine, bovine, caprine,
canine, feline, swine or ovine. In one embodiment, the amount of the TLR7 agonist is about 0.01 to 100 nmol,
about 0.1 to 10 nmol, or about 100 nmol to about 1000 nmol. In one embodiment, the amount of the TLR4
agonist is about 2 to 20 umol, about 20 nmol to 2 umol, or about 2 umol to about 100 umol. In one
embodiment, the ratio of TLR7 to TLR4 agonist is about 1:10, 1:100, 1:200, 5:20, 5:100, or 5:200. In one
embodiment, the composition is injected. In one embodiment, the liposomes comprise DOPC and cholesterol.
   In one embodiment, the immunogen is a cell, protein or spore. In one embodiment, the immunogen is administered before or after the composition. In one embodiment, the administration is effective to prevent a microbial infection. In one embodiment, the composition is intranasally administered. In one embodiment, the composition is intradermally administered.

In one embodiment, a pharmaceutical formulation comprising liposomes, a TLR4 agonist and a TLR7 agonist is provided. In one embodiment, the liposomes comprise 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof: 1,2-distearoyl-sn-glycero-3-phosphocholine (DSPC), 1,2-dioleoyl-sn-glycero-3-phosphoethanolamine (DOPE), cholesterol, or a mixture thereof. In one embodiment, the liposomes comprise DOPC, cholesterol or combinations thereof. In one embodiment, the amount of the TLR7 agonist is about 0.01 to 100 nmol, about 0.1 to 10 nmol, or about 100 nmol to about 1000 nmol. In one embodiment, the amount of the TLR4 agonist is about 2 nmol to 20 umol, about 20 nmol to 2 umol, or about 2 umol to about 100 umol. In one embodiment, the ratio of TLR7 to TLR4 agonist is about 1:10, 1:100, 1:200, 5:20, 5:100, or 5:200. In one embodiment, the TLR7 agonist comprises a compound of Formula (I). In one embodiment, formula (I) comprises

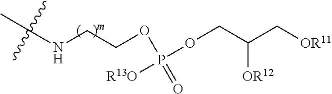

wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof. In one embodiment, m is 1. In one embodiment, R¹¹ and R¹² are each oleoyl groups. In one embodiment,
the phospholipid of R³ comprises two carboxylic esters and each carboxylic ester includes one, two, three or four sites of unsaturation, epoxidation, hydroxylation, or a combination thereof. In one embodiment, the phospholipid of R³ comprises two carboxylic esters and the carboxylic esters of are the similar or different. In one embodiment, each carboxylic ester of the phospholipid is a C17 carboxylic ester with a site of unsaturation at C8-C9. In one embodiment, each carboxylic ester of the phospholipid is a C18 carboxylic ester with a site of unsaturation at C9-C10. In one embodiment, X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. In one embodiment, X² is C(O),

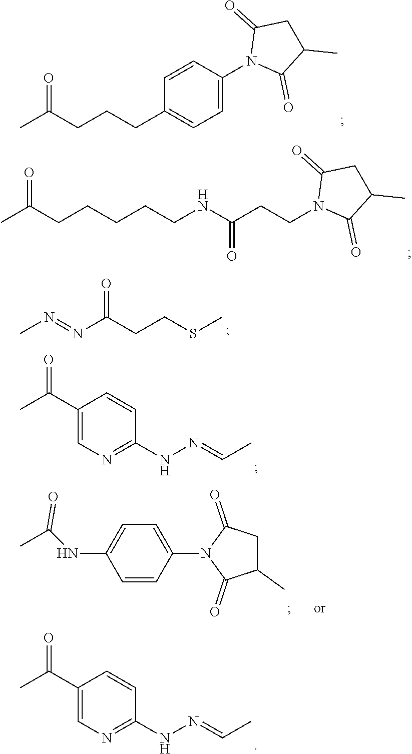

In one embodiment, R³ comprises dioleoylphosphatidyl ethanolamine (DOPE). In one embodiment, R³ is 1,2-dioleoyl-sn-glycero-3-phospho ethanolamine and X² is C(O). In one embodiment, X¹ is oxygen. In one embodiment, X¹ is sulfur, or —NR^{c}— where R^{c} is hydrogen, C₁₋₆ alkyl or substituted C₁₋₆ alkyl, where the alkyl substituents are hydroxy, C₃₋₆cycloalkyl, C₁₋₆ alkoxy, amino, cyano, or aryl. In one embodiment, X¹ is —NH—. In one embodiment, R¹ and R^{c} taken together form a heterocyclic ring or a substituted heterocyclic ring. In one embodiment, R¹ and R^{c} taken together form a substituted or unsubstituted morpholino, piperidino, pyrrolidino, or piperazino ring. In one embodiment, R¹ is a C1-C10 alkyl substituted with C1-6 alkoxy. In one embodiment, R¹ is hydrogen, C₁₋₄alkyl, or substituted C₁₋₄alkyl. In one embodiment, R¹ is hydrogen, methyl, ethyl, propyl, butyl, hydroxyC₁₋₄alkylene, or C₁₋₄alkoxyC₁₋₄alkylene. In one embodiment, R¹ is hydrogen, methyl, ethyl, methoxyethyl, or ethoxyethyl. In one embodiment, R² is halogen or C₁₋₄alkyl, or R² is absent. In one embodiment, R² is chloro, bromo, methyl, or ethyl, or R² is absent. In one embodiment, X¹ is O, R¹ is C₁₋₄alkoxy-ethyl, n is O, X² is carbonyl, and R³ is 1,2-dioleoylphosphatidyl ethanolamine (DOPE). In one embodiment, the compound of Formula (I) is:

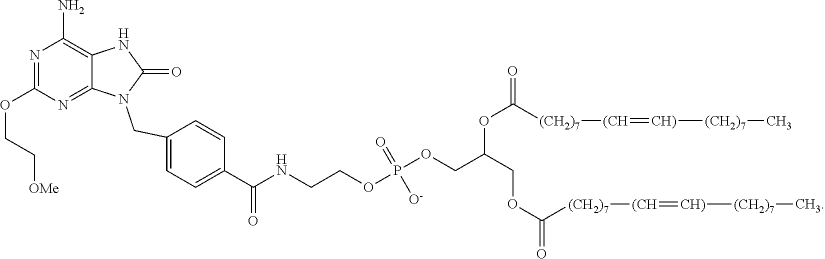

In one embodiment, the compound of Formula (I) is

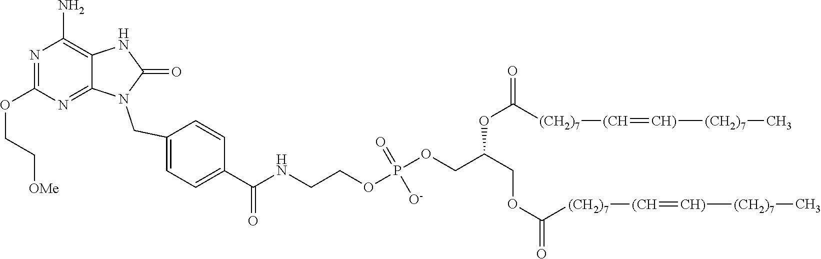

In one embodiment, in formula (II), z2 is 1, 2 or 3. In one embodiment, in formula (II), z1 is 1 or 2. In one
embodiment, in formula (II), z1 is 0. In one embodiment, in formula (II), R⁵ is substituted or unsubstituted aryl
or heteroaryl, e.g., unsubstituted C5 or C6 aryl. In one embodiment, in formula (II),
R⁶ is substituted or unsubstituted cycloalkyl or heterocycloalkyl, e.g., a 5, 6 or 7 cycloalkyl. In one
embodiment, in formula (II), R⁷ is substituted or unsubstituted alkyl, e.g., a C1 to C5 alkyl. In one embodiment, in formula (II), R⁸ is a substituted or unsubstituted aryl or heteroaryl, e.g., a 5, 6 or
7 heteroaryl such as furanyl, pyrrolyl or imidazolyl.

The invention will be further described by the following non-limiting examples.

### Example 1

### Adjuvant Potency of Liposome-Formulated 2B182c, TLR4 Agonist, and 1V270, TLR7 Agonist

The liposomal formulation of 2B182c (200 nmol/injection) and 1V270 (1 nmol/injection) alone or the combination of 200 nmol 2B182c and 1 nmol 1V270 were prepared (Inimmune Corp, Missoula, Mont.). The adjuvant potency of liposome-formulated adjuvants was compared to the DMSO formulation (10% DMSO). The formulated adjuvants were tested using the same protocol. In brief, female BALB/c mice were immunized on days 0 and 21 with liposome-formulated 2B182c (200 nmol/injection) and/or 1V270 (1 nmol/injection) with inactivated influenza virus and sera were evaluated for anti-HA and anti-NA antibodies (IgM, IgG1 and IgG2a) by ELISA. Inguinal lymph nodes were harvested and analyzed for B cell populations by FACS to see whether formulated agonists affect germinal center B cell and plasmablast (antigen secreting cell) populations.

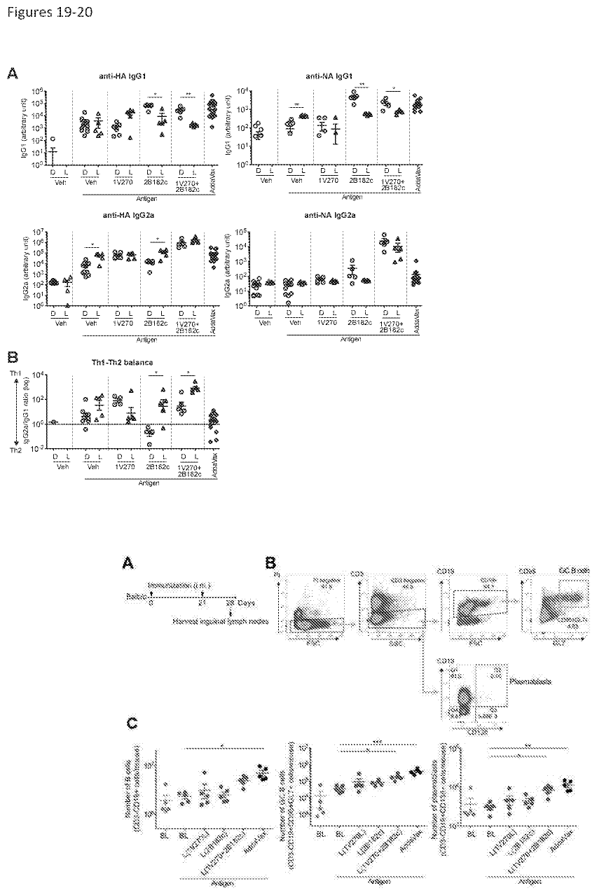

TLR4 are located both on the cell surface and in the endosomal compartment. The signaling through the endosomal receptors inhibits NF-κB activation by LPS. Endosomal TLR4 activation triggers TRIF pathway activation, leading type 1 IFN release through IRF3 activation. Therefore, the adjuvant activity of 2B182c might be attenuated by liposomal formulation. Liposome-formulated 2B182c induces significantly higher anti-HA IgG2a, while liposomal 2B182c reduced HA and NA specific IgG1 in mice immunized with 2B182c alone or 2B182c plus 1V270, in comparison with DMSO formulated adjuvants (FIG. 19A). The liposomal formulation did not affect IgG2a levels in 2B182c and 2B182c plus 1V270 combined adjuvant (FIG. 19A). The decreased levels of IgG1 by liposomal formulation attributed to Th skewing immune responses by liposome-formulated 2B182c and the 2B182/1V270 combined adjuvants (FIG. 19B). These data are consistent to the report that described intracellular delivery of TLR4 ligand induces effective Th1 immune responses dependent to type 1 IFN dependent manner.

After the antigen exposure, activated naïve and memory B cells are expanded and maturated in the germinal center (GC). Maintenance of high antigen specific Ab titers required for long-term vaccine efficacy is correlated with the GC formation. Activated B cells further form antigen-specific Ab-secreting cells (ASCs; plasmablasts and plasma cells), memory B cells and other subsets. Plasmablasts were induced after the seasonal influenza virus vaccination and peak sharply on day 7 post-vaccination. Frequency of plasmablast in peripheral blood after the vaccination with an inactivated virus correlates with the magnitude of protective hemagglutinin inhibition titles in humans. Thus, GC B cells and plasmablasts in the draining lymph nodes were examined. The number of germinal center B cells and plasmablasts were increased by the combination with liposomal 2B182c and 1V270 (FIG. 20).

In summary, liposome-formulated TLR4 and TLR7 ligands adjuvant induced Th1 skewed immune responses and increased GC center B cells and plasmablasts. To evaluate the quality of B cell responses induced by the combined adjuvant in liposomal formulation, we are currently conducting BCR and TCR repertoire analyses of the lymph node cells. Furthermore, the functional evaluation of vaccine adjuvant is evaluated by the live virus (homologous and heterologous challenge).

### Example 2

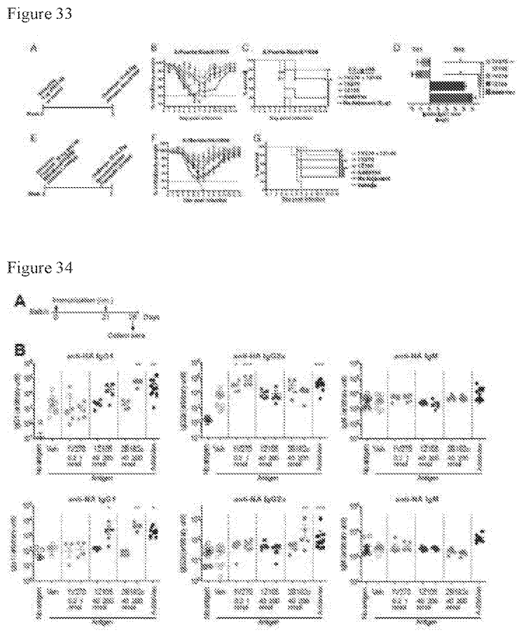

A combination of synthetic small molecule TLR4 and TLR7 agonists is a potent adjuvant for recombinant influenza virus hemagglutinin, inducing rapid and sustained immunity that is protective against influenza viruses in homologous, heterologous, and heterosubtypic murine challenge models. However, the TLR4 agonist used in those studies was 1Z105, a first-generation lead synthetic TLR4 agonist in the pyrimidoindole class that was optimized from hits identified in a high throughput screening campaign to discover adjuvants that act as innate immune receptor agonists. 1Z105 was found to have good immunoactivity in murine cells, but was devoid of significant activity in human cells. In more recent studies, a second-generation series of compounds that contained a C8-aryl substituent was more potent than 1Z105 in murine cells, but was also very active in human cells as well. Within this active group of C8-aryl derivatives, the C8-furan-2-yl derivative (2B182C) was selected for further study based on potency and favorable preliminary formulation data (FIGS. 34 and 36). The pyrimidoindole 2B182C was evaluated in combination with 1V270 for comparison. A MPLA analog (MPLA-1), a potent TLR4 agonist, demonstrated good protection against homologous and heterologous flu challenge in vivo.

The TLR7 agonist, 1V270, is a phospholipid conjugate of a known TLR7 agonist. Major advantages that are conferred by the phospholipid moiety of the agonist conjugate over the corresponding unconjugated agonist include greater potency and lack of local or systemic toxicity, often observed as cytokine syndromes. These favorable properties demonstrating efficacy and safety support the selection of 1V270 as the lead TLR7 agonist for combination adjuvant studies described in this technical proposal.

As mentioned previously, the combined adjuvants comprising the TLR4 agonist 1Z105 and TLR7 agonist 1V270 induced broadly protective responses with influenza-virus vaccine. SAR studies yielded 2B182C which demonstrated higher agonistic potency than 1Z105 in THP-1 cells and in human and murine primary cells in vitro. The adjuvant potency of 2B182C was examined in vaccination models using inactivated influenza virus [A/California/04/09 (Cal/09)] and compared to 1Z105. These studies were conducted using simple DMSO-water formulations of the TLR agonists.

Combined Adjuvant with TLR4- and TLR7-Agonist Induces Rapid and Broadly-Protective Immune Responses to Influenza Virus Infection

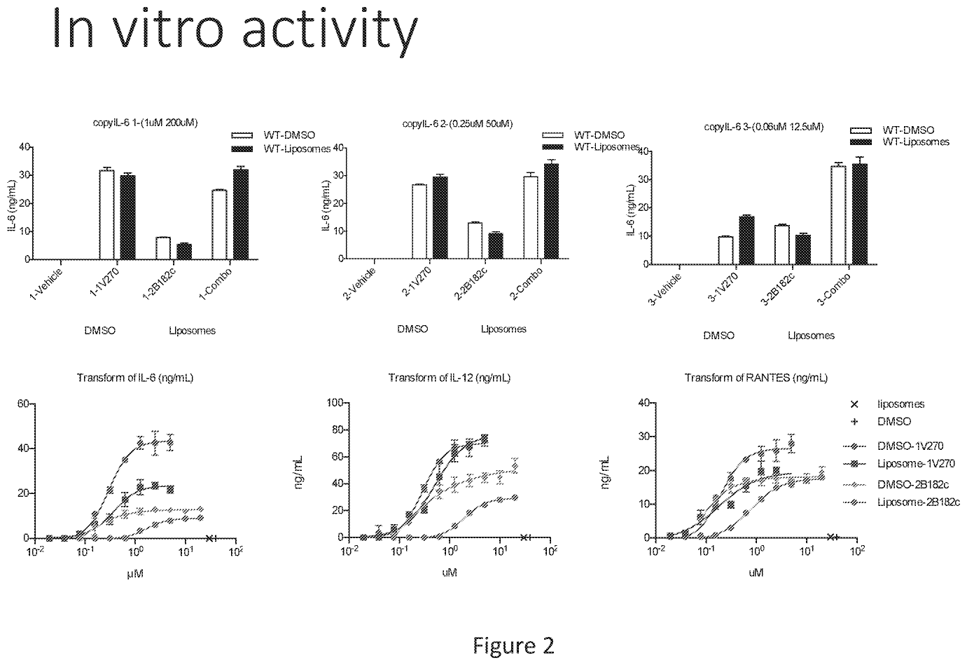

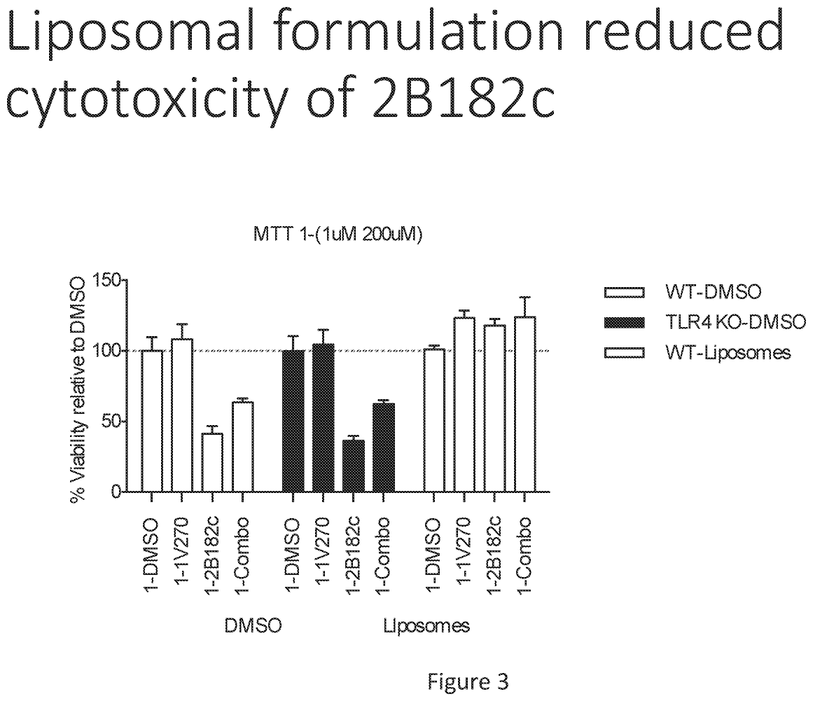

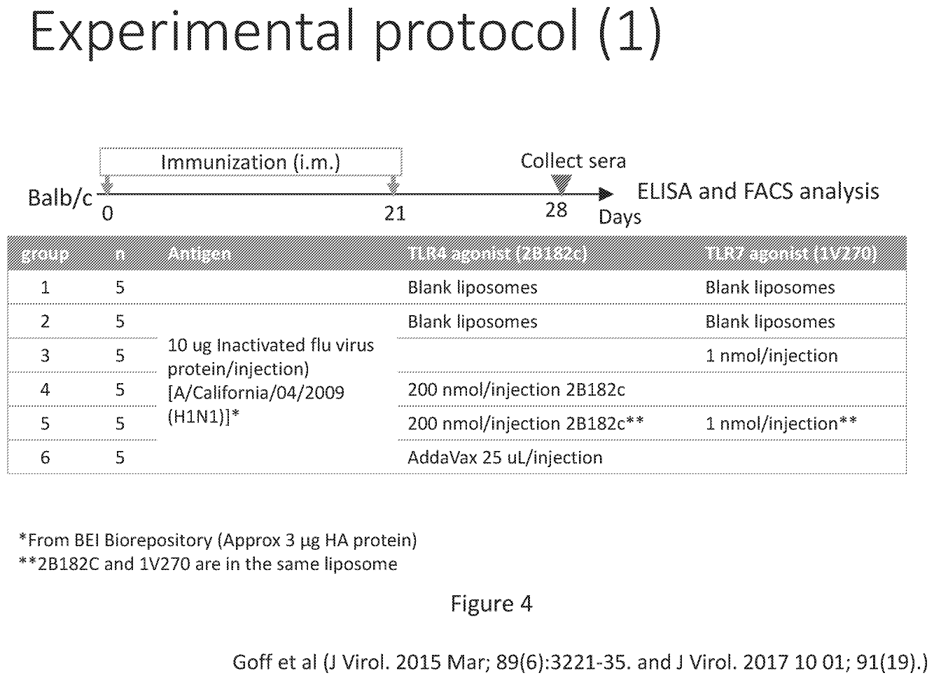

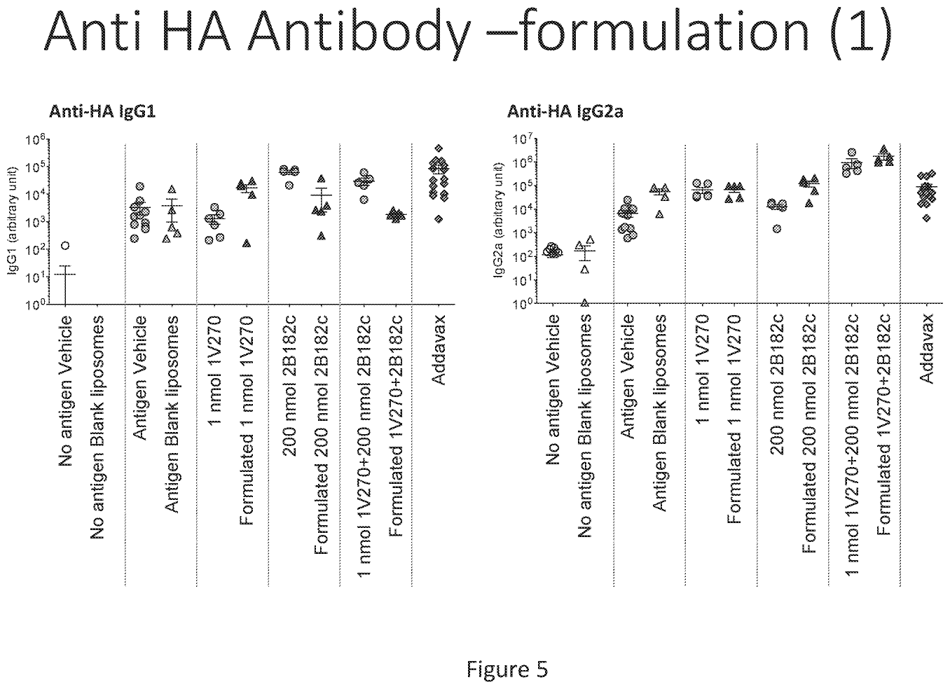

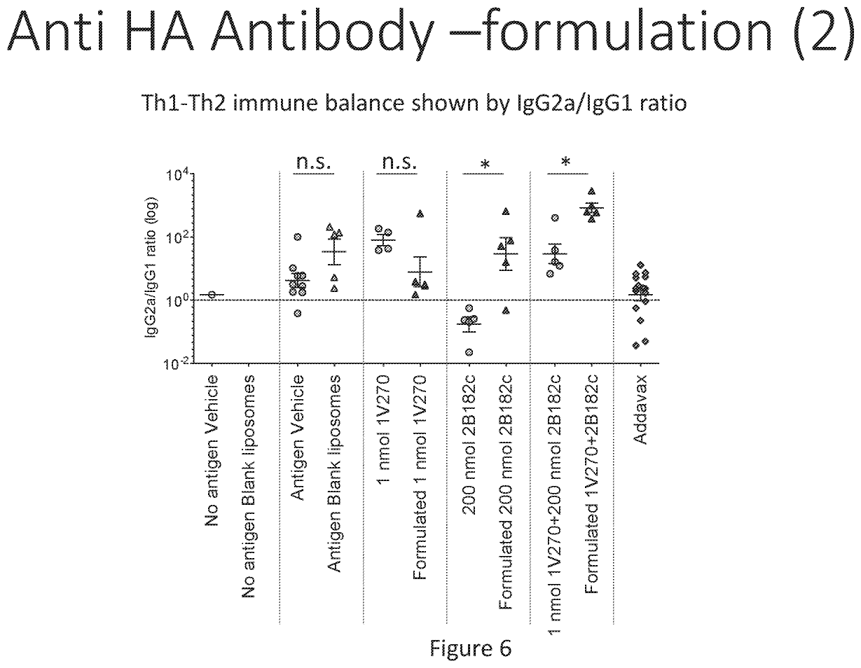

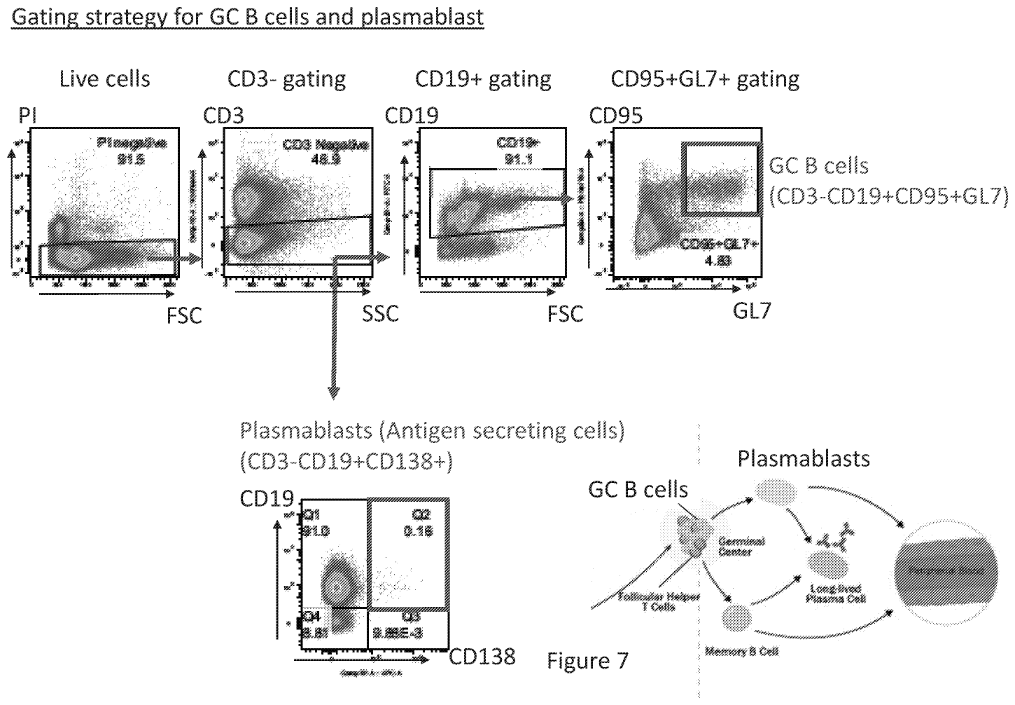

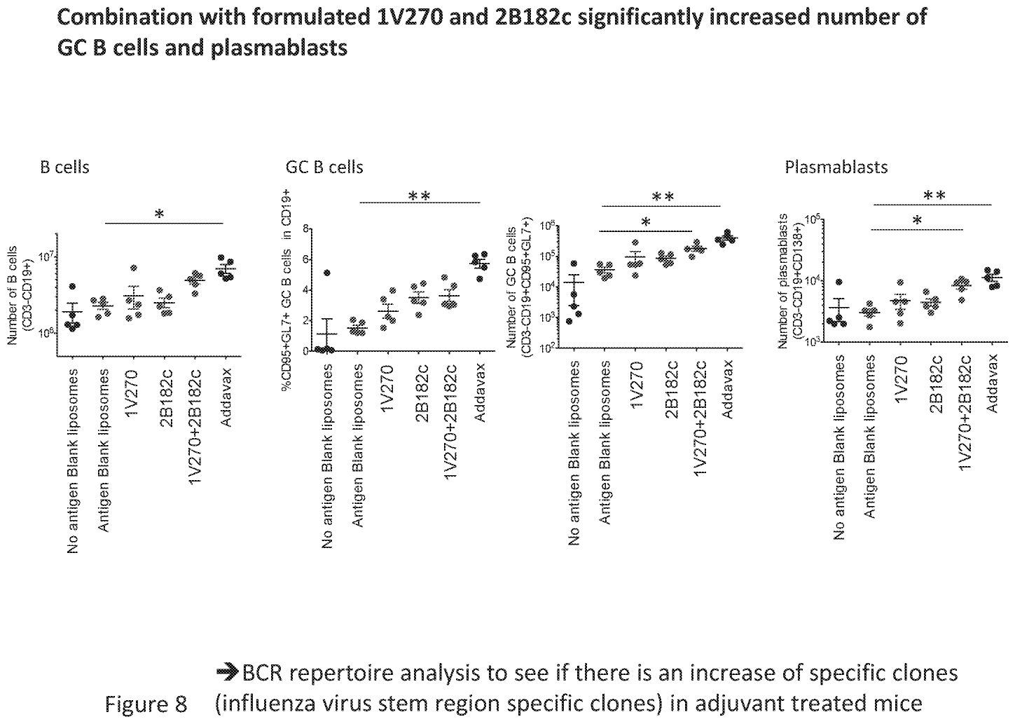

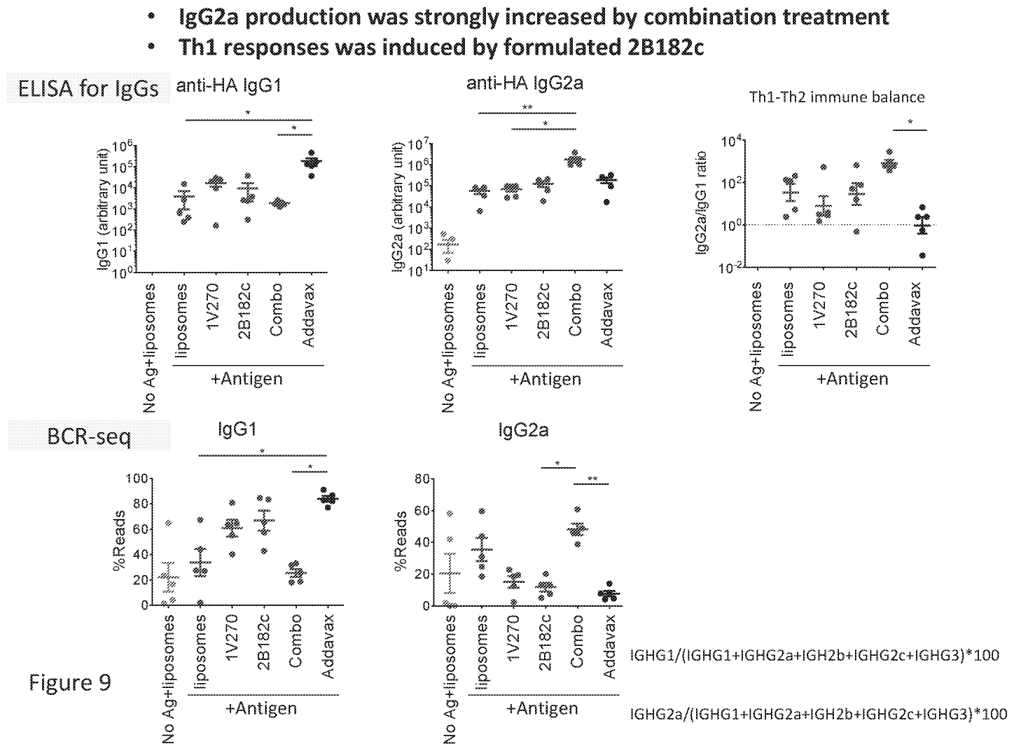

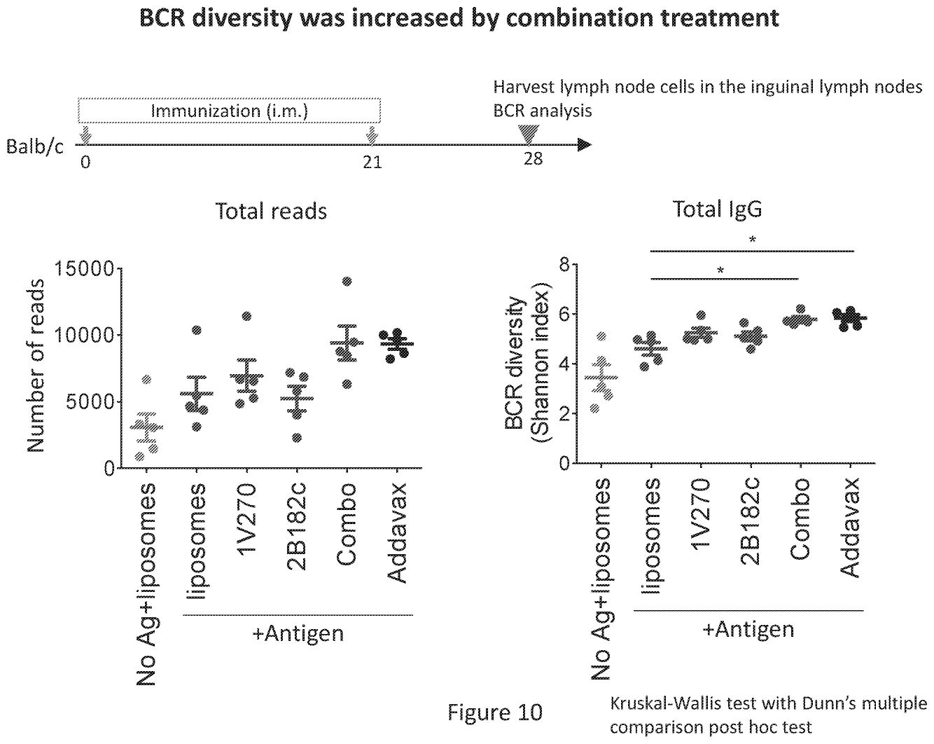

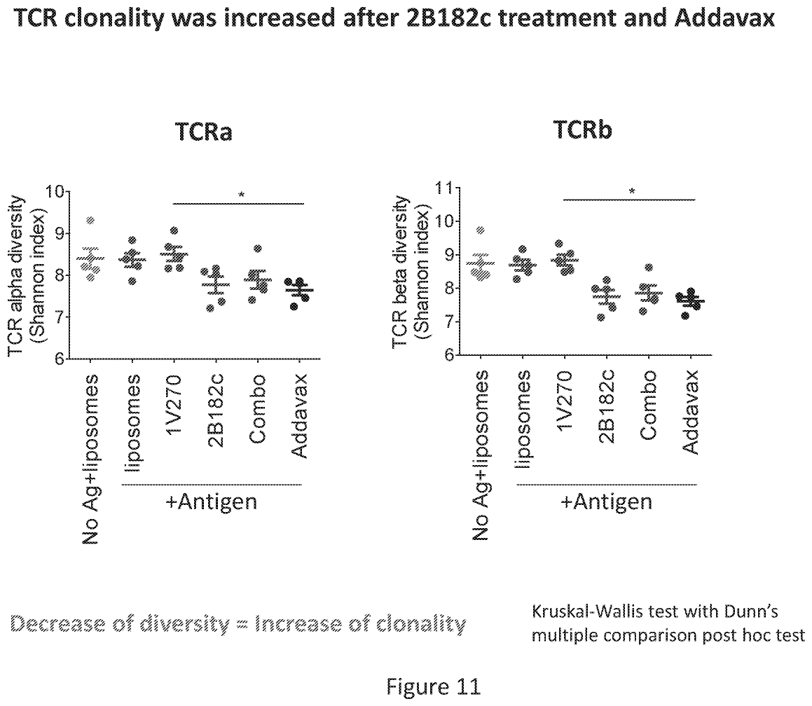

To assess profile of protective immune responses against influenza virus infection induced by TLR4/TLR7 agonist-combined adjuvant, mice were immunized with low dose (0.2 μg/injection) recombinant hemagglutinin (rHA) and humoral responses and protection against lethal virus challenge (FIGS. 34A-2D). The mice immunized with rHA with the combined adjuvant showed minimal body weight loss and higher survival rate (FIGS. 34B and 2C). The combined adjuvant and 1V270, TLR7 agonist, alone induced Th1 biased immune responses.

To further examine whether TLR4/TLR7 combined adjuvant provides cross-protection against heterotypic influenza virus challenge, we immunized mice with 2009-2010 Fluzone, containing B/Brisbane/60/2008 (Victoria lineage), and challenged them with 25 mLD50 of a heterologous mouse-adapted virus B/Florida/04/2006 (Yamagata lineage). More than 90% mice were survived following vaccination of Fluzone adjuvanted with 1V270, alone or in combination with 1Z105 (FIGS. 34E-2G). These data indicated that 1V270, alone or in combination with 1Z105, induces rapid and cross-protective immunity to heterologous influenza viruses.

### Determination of Doses for TLR4- and TLR7-Agonists

As mentioned, SAR study yielded 2B182C that exhibited higher potency in vitro in comparison with 1Z105 in human and mouse immune cells. To examine whether the higher potency observed in vitro study is also reproducible in vivo, female Balb/c mice were intramuscularly (IM) immunized on days 0 and 21 with the TLR4 agonists (1Z105 or 2B182C, 40 or 200 nmol/injection) and 1V270 (phospholipid TLR7 agonist conjugate, 0.2 or 1 nmol/injection) with inactivated influenza virus (A/California/04/2009 (H1N1) pdm09, Cat #NR-49450, BEI resources) (FIG. 34A). The sera were collected on day 28 and anti-hemagglutinin (HA) and anti-neuraminidase (NA) antibodies (IgM, IgG1 and IgG2a) were determined by ELISA. 1V270, 1Z105 and 2B182C were dissolved in DMSO and diluted and the final concentration of DMSO was 10% used as a vehicle control. Data were pooled from four independent experiments showing similar results.

### Effects of TLR Agonist Single Agents on Antibody Secretion

As a single adjuvant, 0.2 nmol and 1 nmol 1V270, and 40 nmol and 200 nmol 2B182C or 1Z105 were compared (FIG. 34B). Both TLR4 agonists, 1Z105 and 2B182C, induced significantly higher levels of IgG1 against HA and NA. Regarding IgG2a induction, both 0.2 and 1 nmol/injection 1V270 significantly increased anti-HA (p<0.05), while only 200 nmol/injection 2B182C, but not 1Z105, enhanced anti-NA Abs (p<0.01) (FIG. 34B). 2B182C and 1Z105 induced similar levels of HA specific IgG2a. There were no differences in IgM response by any adjuvant treatments. These data support reports that TLR4 agonists increased IgG1 production and TLR7 agonist was effective on IgG2a secretion and 2B182C showed similar or modestly higher potency compared to 1Z105 in vivo.

Effects of Combination Treatment with 2B182C and 1V270 on Antibody Secretion

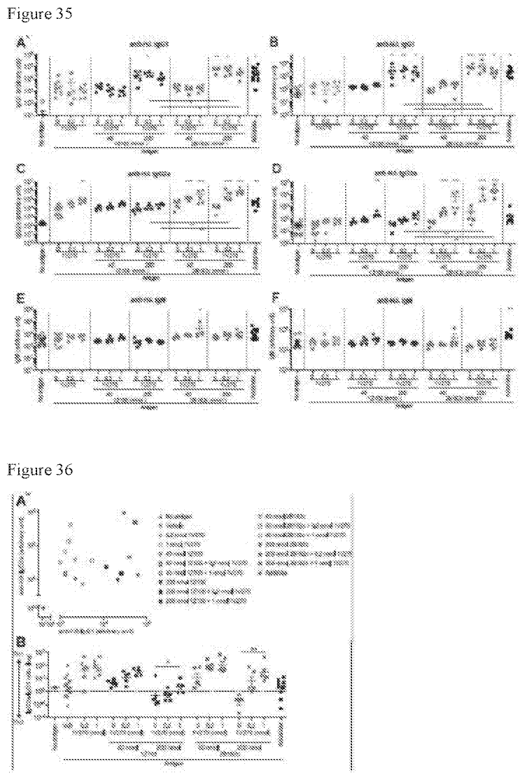

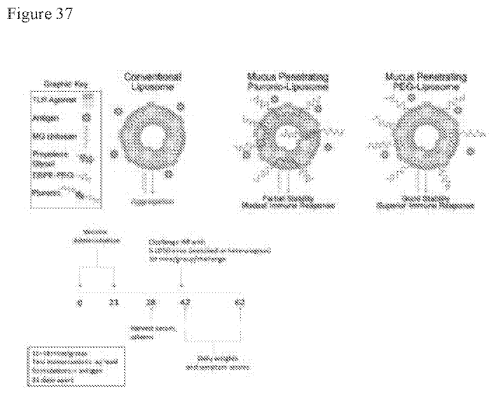

Next, the potency of the combined adjuvants was evaluated using the DMSO-water formulations. Both combined adjuvants of 1V270 with 1Z105 and 2B182C improved induction of IgG1 against both HA and NA. 2B182C enhanced significantly higher IgG1 compared to 1Z105 at both 40 and 200 nmol (p<0.05, FIGS. 35A and 35B). In IgG2a induction, 2B182C increased the levels of anti HA- and anti NA-Abs: however, 1Z105 failed in most cases (FIGS. 35C and 35D). The adjuvants showed minimal effects on IgM release (FIGS. 35E and 35F).

To compare the antibody titers of all combinations tested, the average IgG1 and IgG2a titers are plotted in FIG. 36A. 200 nmol 2B182C plus 0.2 or 1 nmol 1V270 showed the highest inductions of both IgG1 and IgG2a (FIG. 36A). Further, to evaluate Th1/Th2 immune balance, IgG2a: IgG1 ratio was calculated in individual animals (FIG. 36B). 1 nmol 1V270 significantly shifted the Th2-biased immune responses by 1Z105 or 2B182C, indicating that 1V270 shifted immune responses to Th1 bias (FIG. 36B). In summary, these results indicated that the combination of 200 nmol/injection 2B182C plus 1 nmol/injection 1V270 induced the highest quantity of IgG1 and IgG2a and Th1-skewing immune responses are desirable for heterologous protection in the influenza virus infection. Thus, we selected this combination for further preclinical formulation.

Preliminary Data with a TLR4 Agonist

The in vivo evaluation for MPLA-2, a sulfate analog of MPLA, combinations and for all lead TLR agonists in nanoparticle formulations are conducted. A potent TLR4 agonist was discovered during NIAID adjuvant discovery and development contracts where it demonstrated additive if not synergistic enhancement of influenza relevant cytokine production in vitro (in hPBMCs), enhancement of IgG2A antibody and HI titers with 1V270 in vivo in mice and pigs. A major weakness of MPLA-1 as an adjuvant is its lack of chemical stability as it is prone hydrolysis in aqueous media. In a preliminary murine study of non-specific resistance, MPLA-2 protected mice from lethal influenza challenge better than an equivalent dose of MPLA-1 and thus, MPLA-2 represents a next generation TLR4 agonist.

In support of the objectives outlined above the experiments detailed below will be carried out.

### Research Area 1: Formulation and Analytical Assay Development for Lead TLR Agonist Combinations

### Development of Formulations of TLR4/TLR7 Combinations

Task 1A: Development of colloidally stable nanoparticle formulations of lead compounds alone and in combination

Particulate delivery systems act as adjuvants through mimicking the size and shape of the viral and bacterial pathogens our immune systems evolved to recognize and combat via pattern recognition receptors (PRRs). Research over the past 30 years has brought about numerous nano and microparticle based systems that are biodegradable and suitable for vaccine antigen delivery. Their utility as vaccine delivery systems has been demonstrated in the literature with liposomes, virosomes, Iscoms, emulsions, virus-like-particles (VLPs), solid-lipid-nanoparticles (SLNs) and polylactic co-glycolic acid (PLGA) polymers, with examples of each type advancing to human clinical trials. The primary adjuvant mechanism of particulate delivery vehicles is thought to be enhanced uptake of particle incorporated or associated antigens by APCs. It is now well established that the addition of PAMPs to antigens facilitates a robust innate and adaptive immune response through ligation of TLRs and other PRRs leading to innate immune cell activation. A number of PAMPs (bacterial lipoproteins, glycolipids, DNA and viral RNA etc.) have been identified and isolated from viral and bacterial pathogens. Many of these agonists are powerful adjuvants, but exert an unacceptable level of inflammation or have unfavorable physical/chemical characteristics for clinical development. In response, researchers have successfully produced synthetic analogs with improved safety and chemical profiles and many of these have been added to particulate delivery systems to enhance their pathogen mimicry through PRR ligation. Particulate delivery systems can also be used to improve the biodistribution kinetics of adjuvants in vivo and reduce adjuvant side effects without sacrificing adjuvant immunogenicity.

The effective sublingual vaccine use of PEGylated liposomes with bilayer incorporated TLR4 agonist MPLA-1 has been shown in murine models of influenza. This formulation reduces the pyrogenicity of MPLA-1 200-fold without any loss of adjuvant potency in vivo. This is analogous to the observed reduction of pyrogenicity of LPS when incorporated in liposomes versus aqueous dispersions. The same reduction of pyrogenicity is expected for TLR4 agonists.

A number of different lipids and components for the nanoparticle/microparticle formation, API incorporation, API stability and colloidal stability were evaluated. A range of commercially available cationic (DDA, DOTAP, DC-cholesterol), anionic (DPPG, PS, POPG) and neutral lipids (PC, DOPC, DSPC) are tested with the TLR4 and TLR7 agonists. Other formulations may employ PLGA, polycaprolactone, poly(propargyl methacrylate) or PLMA. Because particle size and charge have been shown to significantly influence nanoparticle uptake and processing by DCs, the impact of these variables to enhance delivery vehicle design for the quality characteristics listed above is explored. Small-scale liposomal formulations can be prepared using a thin-film method adapted for sterile serum vials to further reduce scale and waste.

Briefly, this will be done by:

1. Adding APIs to the lipid and dissolving them in chloroform (a fluorescent marker may also be added at this step if desired, e.g. NBD, BODIPY, FITC, etc.)
2. Rotary evaporation at a set speed and vacuum to a dry thin-film
3. Rehydration with aqueous buffer (0.1M phosphate, TRIS or HEPES)
4. Particle size reduction by bath sonication above the lipid transition temperature (Tm) with in process monitoring of particle size, polydispersity and surface charge (zeta potential) by dynamic light scattering (DLS)
5. 3 to 10 mL scale lots of lead formulations will be prepared using a Lipex extruder which improves particle size homogeneity (polydispersity index, PDI) over sonication methods.

### Task 1B: Stability Studies to Assess Colloidal and Physical Stability of Formulations

Formulation stability is needed for development of a successful commercial product as it impacts product storage, shipping and shelf life which all directly contribute to product cost. Formulations are demonstrated to be suitable as potential products, as well stability, particularly when selecting lead candidates to pursue further.

Lead formulations are assessed for short term accelerated (25 and 40° C.) and long-term real time stability (2-8° C. and 25° C.) to ensure formulations chosen provide sufficient stability for potential product development (minimum of 12 months at preferred storage condition).

Accurate quantitation of adjuvant incorporation into a nanoparticle delivery system is essential for proper dosing, vaccine efficacy and safety. SEC-HPLC and RP-HPLC methods for quantitation of TLR4 and TLR7/8 agonists incorporated into nanoparticles, including liposomes, were developed. RP-HPLC is effective for analysis of total agonist content present in a nanoparticle when the sample is dissolved with a water miscible organic solvent with sufficiently low background UV absorbance (methanol, tetrahydrofuran, etc.). Dissolution with organic solvent disrupts the nanoparticle and releases any incorporated or surface bound agonist for accurate quantitation by RP-HPLC against a 5-point standard curve.

For quantitation of liposome incorporated (bilayer or aqueous core) agonist, a method capable of analyzing intact liposomes and the extra-liposomal aqueous phase is needed. A SEC-HPLC method able to quantitate “free” TLR agonist with UV detection at 296, 225, and 310 nm (for 2B182C, MPLA-2, and 1V270, respectively) was employed. The TSK gel SWxI series columns provide excellent size-based resolution for nanoparticle formulations in the 30-200 nm range. The mobile phase used is the same as the buffer utilized for the liposome rehydration to maintain a constant osmotic potential between the extra liposomal fluid and the aqueous phase in the liposome core. This method qualified as a complementary method to the in vitro potency assay, which only detects aqueous unincorporated TLR4 agonist.

A preliminary study was conducted using these analytical methods to assess liposomal formulations of 2B182C and 1V270, each prepared alone and in combination (co-encapsulated). The work flow was performed as follows:

1) Lead adjuvant formulation screening (pharmaceutically acceptable co-solvents, excipients, liposomes) on a 2 mL scale with target concentrations of 1 nmol 1V270 and 200 nmols for 2B182C contained in 50 uL for IM injection use.
2) Perform basic analytical method development and analysis on lead formulations to ensure formulations meet quality criteria
3) Evaluate stability of preferred formulations by real-time and accelerated methods, adding appropriate excipients as stabilizers if necessary
4) rFC testing to ensure no endotoxin contamination of finished formulations.

All liposomal formulations were prepared on a 2 mL scale for compounds 2B182C and 1V270.

Briefly, the following procedure was used to prepare the liposomes and the following compositions were evaluated: 2B182 with and without 1V270 using (DOPC/with and without cholesterol, 2:1, respectively). The concentration of DOPC tested was held constant at 40 mg/mL, which resulted in a cholesterol concentration of 10 mg/mL. The liposomes were produced following the lipid film rehydration method using 9:1 Chloroform:Methanol as solvent. The rehydration buffer initially used was 50 mM NaPB, 100 mM NaCl, pH=6.1. The agonist concentrations tested were the target concentrations. Sonication at elevated temperature was used to reduce the liposome particle size. A summary of the analytical results is depicted in Table 4.

Theoretical concentration of prepared formulations:

| | c [mg/mL] | [μM] |
|---|---|---|
| 2B182C | 2.0505 | 4000 |
| 1V270 | 0.0217 | 20 |
| DOPC | 60 | ND |
| Chol | 15 | ND |

Particle size by Dynamic Light Scattering (DLS) at end of sonication and after 8 days at 2-8° C.:

| | | | z-Average_{0d} | PDI₀ | p1₀ | p2₀ | p1₀ | p2₀ |
|---|---|---|---|---|---|---|---|---|
| lot# | Description: | V [mL] | [nm] | [ ] | [nm] | [nm] | [%] | [%] |
| INI020_023_FF_A | 2B182C + 1V270/DOPC/ | 2.1 | 132.1 | 0.45 | 271 | 3831 | 94.1 | 5.9 |
| | Chol/10 mM PB pH = 7.1 | | | | | | | |
| INI020_023_FF_B | 2B182C/DOPC/Chol/ | 2.1 | 255.7 | 0.958 | 1798 | 165.4 | 55.7 | 38.6 |
| | 10 mM PB pH = 7.1 | | | | | | | |
| INI020_023_FF_Blank | DOPC/Chol/ | 4.1 | 114.9 | 0.385 | 129.5 | 2155 | 84 | 16 |
| | 10 mM PB pH = 7.1 | | | | | | | |
| INI020_023_FF_C | 1V270/DOPC/Chol/ | 2.75 | 94.38 | 0.265 | 123.5 | 4227 | 96.6 | 3 |
| | 10 mM PB pH = 7.1 | | | | | | | |
| | | | z-Average_{8d} | PDI_{8d} | p1_{8d} | p2_{8d} | p1_{8d} | p2_{8d} |
| lot# | Description: | | [nm] | [ ] | [nm] | [nm] | [%] | [%] |
| INI020_023_FF_A | 2B182C + 1V270/DOPC/ | | 143.8 | 0.505 | 515.8 | 69.43 | 56.8 | 36.3 |
| | Chol/10 mM PB pH = 7.1 | | | | | | | |
| INI020_023_FF_B | 2B182C/DOPC/Chol/ | | 273.5 | 0.966 | 2506 | 178.9 | 54.4 | 45.6 |
| | 10 mM PB pH = 7.1 | | | | | | | |
| INI020_023_FF_Blank | DOPC/Chol/ | | 114.9 | 0.38 | 145.7 | 3630 | 90.1 | 9.9 |
| | 10 mM PB pH = 7.1 | | | | | | | |
| INI020_023_FF_C | 1V270/DOPC/Chol/ | | NA* | NA* | NA* | NA* | NA* | NA* |
| | 10 mM PB pH = 7.1 | | | | | | | |

TABLE 5
Analysis of preliminary liposomal formulations of 2B182C and 1V270
demonstrating the thorough analytical characterization of lead formulas.
Quantitration by RP-HPLC:

| | | | | [symbol] | [symbol] |
|---|---|---|---|---|---|
| | | c(28182c) | c(1V270) | c(28152c) | c(1V270) |
| lot # | Description | [mg/ml] | [mg/ml] | [mg/ml] | [mg/ml] |
| [symbol] _023_FF_A | [symbol] /ChoI/ [symbol] | 1.9082 | 0.0207 | 2.051 | 0.022 |
| [symbol] _023_FF_B | [symbol] /ChoI/ [symbol] | 2.0079 | NO | 2.061 | 0 |
| [symbol] _023_FF_Blank | DOPC/ChoI/ [symbol] | NO | NO | 0 | 0 |
| [symbol] _023_FF_C | 1V270/DOPC/ChoI/ [symbol] | NO | 6.0217 | 0 | 0.022 |

[symbol]

indicates data missing or illegible when filed

Other ratios of the TLR agonists, other lipid components, and varying amounts of cholesterol for nanoparticle formation are evaluated. At least 10 different formulations are prepared and screened for suitability in the process under Task 2A and compared to the results we obtained using the simple DMSO-water formulations described above.

Nanoparticles: TLR7 and TLR4 agonists are prepared as nanoparticle formulations (liposomes, SLNs, PLGA, emulsions, etc.). The final lead formulations are selected based on immunology, stability and manufacturing data. DOPC/cholesterol liposomal formulations appear to be very promising based on preliminary immunology and stability data. One of the challenges expected with the TLR4 and TLR7 agonists is the co-incorporation of both agonists in the same nanoparticle in a controlled and consistent manner. The ratio of agonists to each other is fixed once co-encapsulated, so any dose adjustment at that point alters both agonists together.

Analytical Methods: All of the analytical methods described in Task 1C have been used with our lipidated TLR-7/8 agonists and TLR4 agonists and we expect to further improve their specificity, linearity and range with additional optimization. The RP-HPLC methods for quantitation of adjuvant in TLR4 and TLR7 agonist formulations will be optimized for peak shape, LOD and LOQ. These same methods will be gradient- and column-optimized to achieve baseline resolution and optimal LOD/LOQ for any degradants detected from stability studies to permit accurate monitoring of product stability. Accurate quantitation of the nanoparticle incorporation percentage for each agonist of the TLR4 and TLR7 agonist combinations could prove challenging since SECHPLC separates based on hydrodynamic volume only. Liposomes and unincorporated agonist may have similar particle sizes, which would limit the utility of SEC-HPLC for incorporation determination. This is discussed below in alternative approaches.

Alternative Approaches: If development of co-encapsulated TLR4 and TLR7 agonists proves to be too difficult due to inconsistent levels of agonists in the nanoparticles, our immunology data has shown that adjuvant synergy can still be achieved by simply admixing TLR4 agonist in liposomes with TLR7 agonist in liposomes. This approach has the potential to produce a simpler, reliable product whose analytical characterization would be made easier by reducing the likelihood for interference of the agonists' signals with one another.

Another option is to explore other formulations for co-encapsulation such as nano-emulsions where 100% of the agonist is incorporated by default because the aqueous and oil phases are mixed into nano-droplets. Emulsions also have the advantage of forming a depot at the site of administration, which can further enhance immune response. As discussed in Research Area 2, co-encapsulated TLR4 and TLR7 agonists versus admixed are compared in vitro and in vivo to weigh the pros and cons of these approaches. An alternative approach to using SEC-HPLC for determination of agonist incorporation into nanoparticles would be high-speed density gradient centrifugation to pellet the nanoparticles and analyze the supernatant for unincorporated agonists using established RP-HPLC methods.

Formulations in the target ratio range that have acceptable properties for advancement are subjected to in vivo studies, including immunization and virus challenge studies.

Research Area 2: Establish the Immunological Biomarkers of Protection from Lethal Influenza Virus Challenge by Lead Adjuvant Formulations

Defining reliable biomarkers is needed for successful development of safe and effective vaccines. Selection of vaccine candidates with a profile that effectively prevents the infection without any safety issues is essential for the vaccine development program. In a vaccine clinical trial, identification of biomarkers that predict antigen-specific adaptive immune response with minimal reactogenicity is required. In this project, biomarkers are identified in two steps, 1) Innate immune biomarkers induced by the formulated lead adjuvant with and without antigen, and 2) Biomarkers correlating to adaptive immune responses. Thus, in vitro and in vivo studies are performed to identify the biomarker candidates that correlate to biologic activities of both TLR4 and TLR7/8 ligands and that also relate to reactogenicity.

### Task 2A: Combination Formulations Based on In Vivo Antibody Production Studies for Immunoactivity and Reactogenicity

The hallmark of protection from infectious disease through vaccination is the induction of effective antibody production. Combining TLR4 with TLR7 agonists resulted in significant increases in antigen-specific antibody titers. A trend toward Th1 biasing of the immune response was observed. The effectiveness of the formulated adjuvants and their combinations is compared to the simple DMSO-water preparations.

### Task 2A.1: Immunization Studies in Mice for Lead Combo Formulations

Formulations of lead adjuvants will be evaluated in immunization studies alone and in combination at various ratios of TLR agonists in a similar manner as previously completed for the DMSO-water formulations. The levels of IgM, total IgG, and IgG1 and IgG2a specific for both HA and NA are assessed. One or more ratios of TLR agonists in combination are identified that provide the maximum titers of antigen-specific antibody. This formulation(s) will be advanced to challenge studies under Research Area 3.

### Task 2A.2: Evaluation of Reactogenicity and Toxicity of Lead Combo Formulations in Mice

Since infectious disease vaccines are designed to be protective in populations of healthy individuals, vaccine safety must be of the highest priority among development goals. Therefore, appropriate experiments to evaluate toxicity and reactogenicity of the candidate formulations are conducted. In these experiments and in general, overt toxicity is closely evaluated as initial toxicity assessments. Signs of any distress in the mice (i.e. lack of grooming, mobility issues, conjunctives, abnormal behavior, responsiveness etc.) will be noted. In addition to the gross observations, toxicity measurements comprise complete blood count, serum chemistry assessments (AST, ALT, ALP, amylase, blood urea nitrogen, creatinine, total protein, glucose, potassium, calcium, sodium, total bilirubin) and necropsy assessments (spleen, liver, and kidney sections stained with hematoxylin and eosin). Furthermore, the injection site is evaluated for visible signs of inflammation and any other abnormal findings. Tissue at the injection site is also evaluated histologically as a part of the necropsy assessments. These studies are summarized in Table 6 below.

Task 2B: Identification of Immune Markers that can Predict Protective Adaptive Immune Responses

As mentioned, identification of biomarkers that predict antigen-specific adaptive immune response with minimal reactogenicity facilitate clinical trials design and methods.

### Task 2B.1: Innate Immune Response Signatures (Cytokines, Chemokines)

Immune cell recruitment to the local vaccine administration site by chemokines is essential to recruit antigen presenting cells (APC) and influence induction of subsequent adaptive immune responses. However, the site of injection, i.e. muscle tissue, contains relatively few immune cells and therefore effective adjuvants must induce recruitment of immune cells to the local site. TLR4, unlike TLR7/8, is abundantly expressed on non-immune cells, able to express sufficient chemokines to recruit the inflammatory cells. Following TLR stimulation, it is difficult to distinguish inflammatory responses from adjuvant effects because recruitment of APCs usually accompanies inflammatory cells. These complex cascades of immune activation cannot be studied in in vitro assays alone. Hence, panels of markers are selected from the above in vitro experiments in the samples obtained from in vivo studies in mice.

The lead adjuvant formulations are administered intramuscularly (IM) to mice, and sera will be collected on days 1, 3 and 7 after injection to examine levels of systemic cytokines/chemokines. As mentioned in Task 2A.2 above for local muscle tissue, expression of cytokines/chemokines and co-stimulatory molecule genes will be examined by qPCR or NanoString assays. Immune cell infiltration is assessed by histologic examination of the selected samples with hematoxylin-eosin staining and immunohistochemical staining. Splenocytes or PBMCs are used to evaluate the expression of co-stimulatory molecules assessed by flow cytometry. The draining lymph nodes are collected at the indicated time points and pooled in each experimental group and analyzed for immune cell populations and expression of chemokine receptors, and costimulatory molecules. A summary of the study design is shown in Table 6. Note that “Group 5: The combined adjuvant with antigen” group, could include combinations of different ratios of TLR4 and TLR7 agonists as necessary to provide desired profiles of cytokine/chemokine induction. Innate immune signatures that show biologic activities of both TLR4 and TLR7 ligands, and that also relate to reactogenicity, are selected.

TABLE 6
Example of study design for innate cytokine/
chemokine markers (mouse)

| | Description |
|---|---|
| Groups | Group 1: Vehicle alone no antigen |
| | Group 2: The combined adjuvant without |
| | antigen |
| | Group 3: Adjuvant (TLR4 ligand) alone |
| | Group 4: Adjuvant (TLR7 ligand) alone |
| | Group 5: The combined adjuvant with antigen |
| | Group 6: FDA approved adjuvant (e.g. MF59) |
| Injection | Day 0 and day 21 |
| Route | IM |
| Evaluation | Day 1, 3, 7, after the last injection |
| # mice | N = 10 per each time point |

### Task 2B.2: Adaptive Immune Response Signatures.

The experiments to assess adaptive immune responses are conducted in conjunction with Task 2A.1 above. Biomarker candidates that satisfy the following criteria are identified: 1) detected in peripheral blood, 2) driven by mechanism of actions of each TLR ligand and correlating their biological effect, 3) predicting long-term antigen-specific antibody induction and broad protection, 4) predicting reactogenicity.

### Outcomes and Alternative Approaches

One or more ratios of TLR agonists in combination provide maximum titers of antigen-specific antibody. Moreover, the use of combinations of the two classes of TLR ligands results in a shift in the adaptive immune response toward a Th1-biased response compared to the use of a TLR4 agonist alone. Thus, the Th1/Th2 response ratio likely increases. This Th1 bias may favor the broadening of the response to include heterologous virus protection. As for toxicity, systemic and oral administration of TLR7/8 ligands of the imidazoquinoline class have shown severe side effects comprising flu-like symptoms, nausea and lymphopenia with high levels of serum TNFα and IL-1β. This may also be true for the oxoadenine class, of which 1V270 is a member. However, most of these undesirable side effects can be avoided by employing the usual local route of administration for vaccinations, IM. Moreover, the TLR7/8 ligand was prepared by conjugation to lipid moieties as well as by customizing the formulation, and successfully reduced the systemic cytokine release while maintaining the adjuvant activity. Thus, because of the low systemic exposure to inflammatory cytokines, there will likely be little or no reactogenicity associated with the lead formulated combinations.

### Research Area 3: Selection of Formulation(s) and Immunization—Virus Challenge Studies in Mice (Inimmune)

Based on results of the formulation studies including stability (Task 1B), immunoactivity (Task 2A.1), and reactogenicity profile (Task 2A.2), the leading formulated combination, along with a backup combination, are selected for the preclinical immunization/virus challenge studies in mice. The virus antigens used for the studies may be selected from either recombinant vaccine antigens or inactivated whole viruses that have been used in licensed commercial vaccines, such as A/Victoria/3/75(H3N2), A/Michigan/45/2015 (H1N1) pdm09-like virus and A/Hong Kong/4801/2014 (H3N2)-like virus.

### Task 3A: Selection of Lead Combo Formulation(s)

Lead selection criteria is based on: 1) stability of formulated combinations, 2) ratios of TLR agonists that provide desired antigen-specific antibody levels, and 3) low reactogenicity profile, both local and systemic. Specific studies related to these criteria are summarized in Table 7.

Following selection of a lead formulated combination and a backup lead combination, evaluation of the selections in immunization/virus challenge models in mice will be carried out (Task 3B).

TABLE 7
Summary of Measurements

| Function | Description |
|---|---|
| Antigen presenting | Flow cytometric assay of CD80, CD83, CD86, CD40, MHC class II expression of |
| function | CD11c positive cells in the draining lymph node cells and splenocytes |
| Inflammatory reaction, | Gene expression study of local tissue at the injection sites |
| cytokine and | Histologic examination of the injection site |
| chemokines | Luminex assay of sera for systemic cytokine and chemokine release |
| B cell analysis | Flow cytometric assay of CD19 [symbol] CD138 [symbol] B cells and |
| (Phase II plan) | CD19 [symbol] CD138 [symbol] plasmablasts in the draining lymph nodes. PBMC and [symbol] cytes |
| | BCR [symbol] analysis of draining lymph node cells. PBMC and [symbol] cytes |
| T cell analysis | Flow cytometric assay for CD69 expression of CD3 [symbol] CD4 [symbol] and CD3 [symbol] CD8 [symbol] cells |
| (Phase II plan) | CD62L [symbol] CCR7 [symbol] CD44 [symbol] (effective memory T cells) |
| | CD62 [symbol] CCR7 [symbol] CD44 [symbol] (central memory T cells) |
| | Multifunction CD4 [symbol] T cells (intracellular staining for TNF, IL-2, IFNγ) |
| | CD4 [symbol] CXCR5 [symbol] ICOS [symbol] PD-1 [symbol] [symbol] T cells |
| | T cell ELISpot (IFNγ) |
| General toxicity | Complete blood count |
| assessments | Serum Chemistry: AST, ALT, ALP, Amylase, blood urea nitrogen, [symbol] , total |
| | protein, glucose, potassium, calcium, sodium total [symbol] |
| | Necropsy (Spleen, liver and kidney sections stained with hematoxylin and eosin) |

[symbol]

indicates data missing or illegible when filed

Task 38: Immunization/Virus Challenge Studies with Lead Formulations

### Task 3B.1: Determination of Minimum Protective Dose for Virus Challenge Studies

Because inactivated influenza virus contains innate immune receptor ligands (PAMPS), a certain low level of protection might be expected following immunization of mice with sufficient antigen alone. Therefore, a study to determine the minimum protective dose, if any, with inactivated virus is conducted. The minimum protective dose of antigen is that dose that provides only partial protection (below 30% survival) upon subsequent challenge with matched strain of active virus. This strategy allows for a range of activity to be observed with the selected lead formulated adjuvant combinations. In addition, the amount of challenge virus can also be confirmed that results in complete mortality for non-immunized mice, typically a dose of about 5 LD50.

### Task 3B.2: Homologous Virus Protection Study

Following the antigen dose range finding study, a mouse model is used to evaluate the immunogenicity of the lead adjuvant combinations along with homologous influenza vaccine antigens. The primary determinants of success are: 1) durable influenza specific IgG2a and IgG1 in the sera. 2) protection from lethal influenza virus challenge, 3) low reactogenicity, and 4) induction of multifunctional CD4+ versus CD8+ T cells as assessed by intracellular IFNγ/TNFα staining. Secondary endpoints include weight gain/loss and a scoring of disease severity through the monitoring of the observable clinical symptoms (ruffled fur, hunched posture and labored breathing) following vaccination or influenza virus challenge.

### General In Vivo Methods

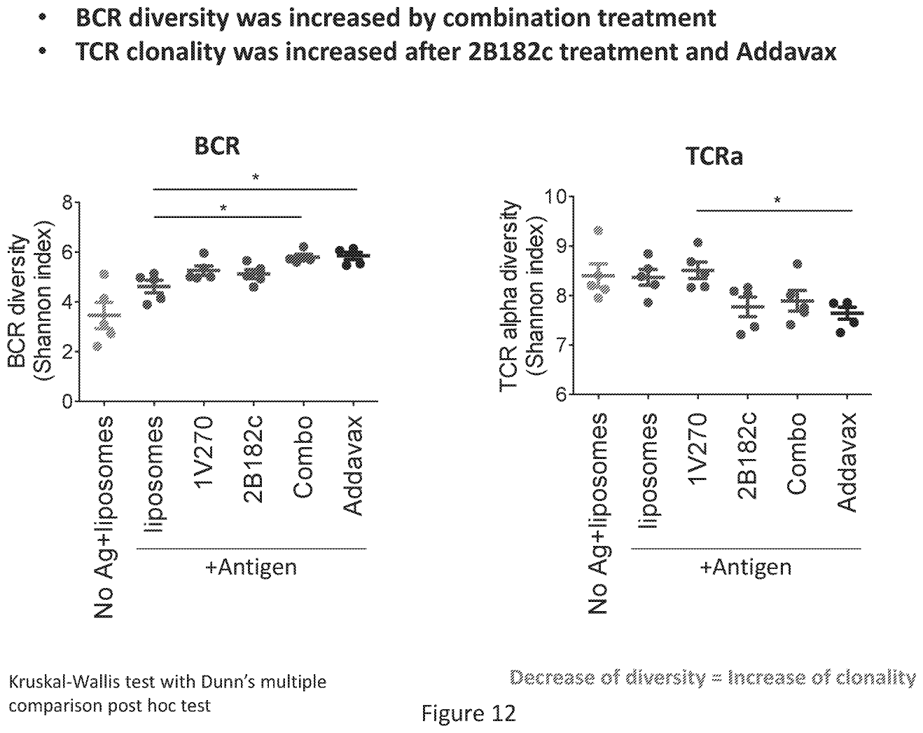

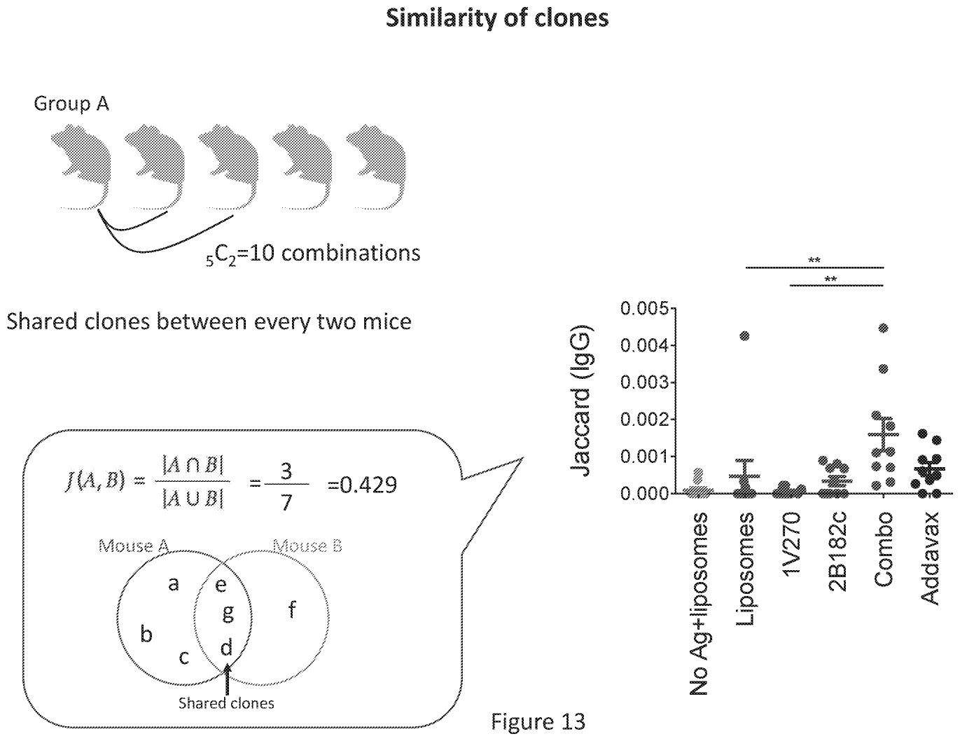

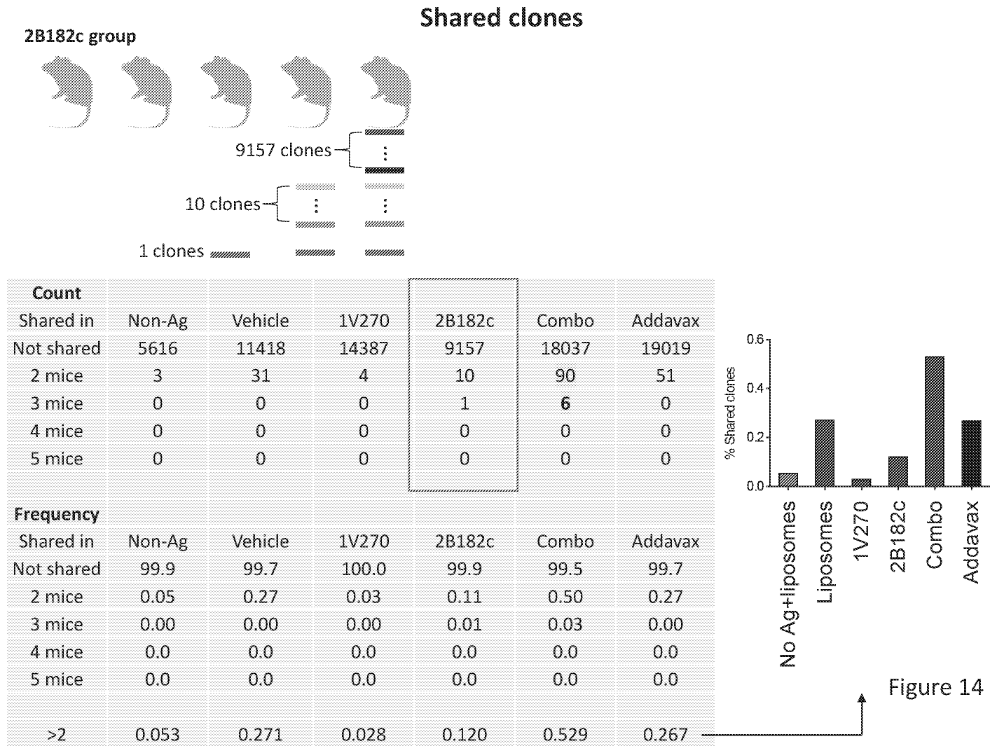

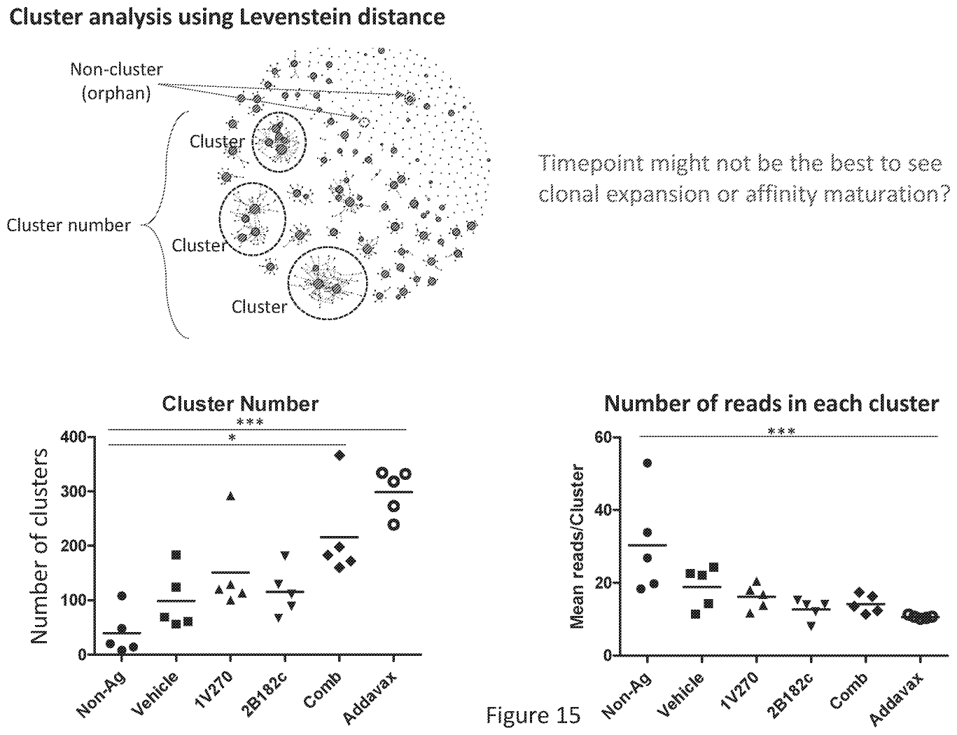

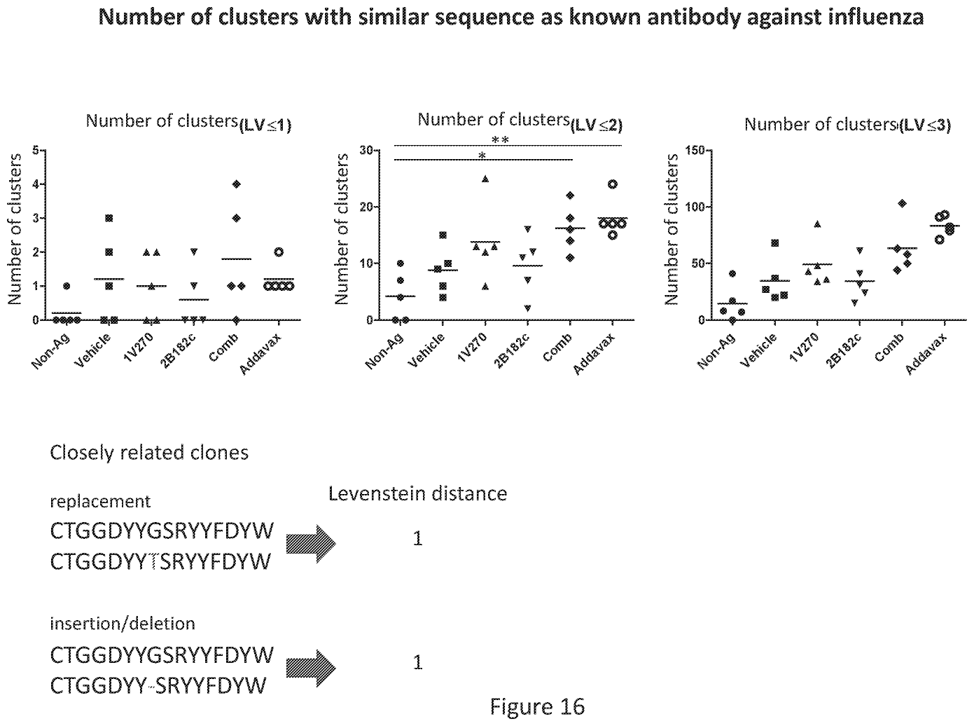

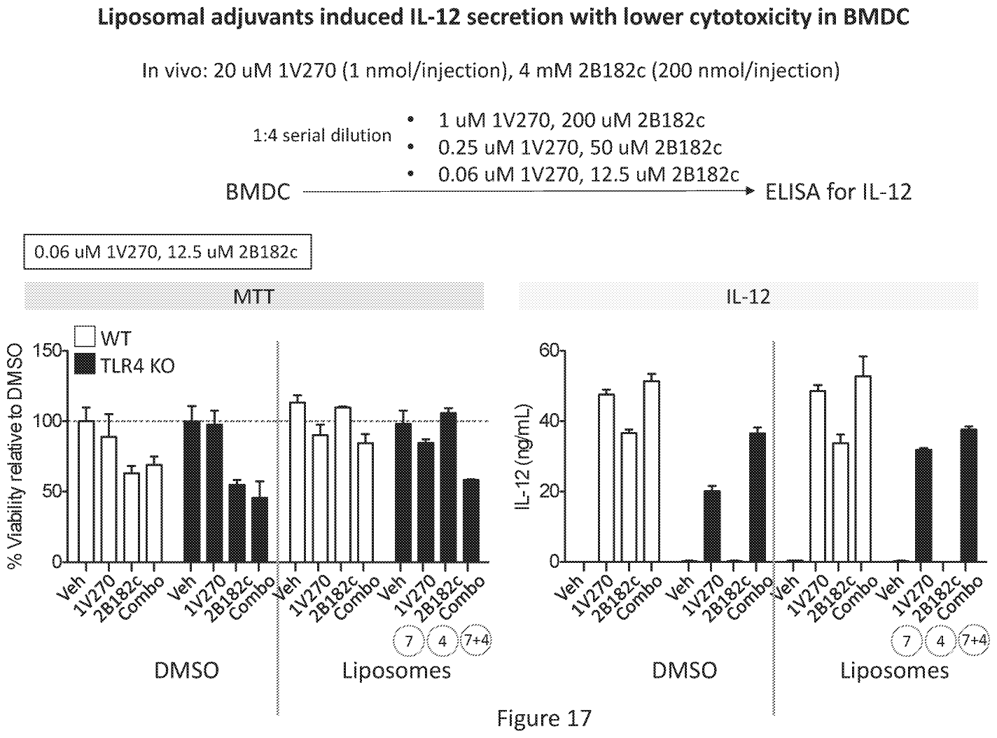

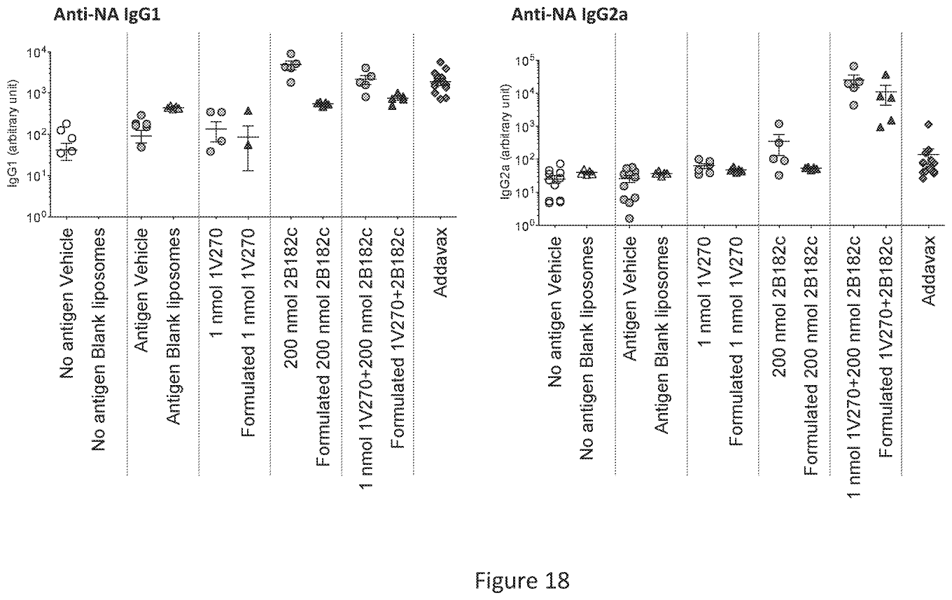

Immunologic evaluation: Mice (male and female) are vaccinated (adjuvant+flu antigen such as A/Victoria/3175(H3N2)) one or two times via IM administration with 21 days between the primary and secondary vaccinations (FIG. 12). Cell-mediated immunity (CMI) is evaluated in a subset of 4 mice per group by measuring Th1/Th2 cytokine induction in splenocyte cultures (assayed by ELISA) and multifunctional CD4+ and CD8+ T-cell responses (assayed by FACS, 10-color intracellular cytokine staining). Further, tetramer staining and cell surface phenotyping are performed to determine the frequency of influenza-specific memory CD4+ and CD8+ T cells. Flu specific humoral responses are measured in serum (IgG1 and IgG2a) and HI titers are used to measure functional antibody titers. Vaccinated and control mice are challenged with 5 LD50 of A/HK/68(H3N2) and assessed for survival, weight gain/loss and a scoring of disease severity for 21 days. Reactogenicity in these murine studies is measured by weight loss and symptom scores and evaluation of injection site infiltrates. A p value difference of <0.05 is considered significant. Analysis of variance (ANOVA) and Tukey ANOVA is performed on all data to demonstrate robust statistical significance.

### Task 3B.3: Heterologous Virus Protection Study

Following the homologous protection study, the same study design is used to evaluate the lead adjuvant combinations in a mouse model of heterologous or heterosubtypic protection. Mice are immunized as described above (Task 3B.2) but are challenged with an influenza virus strain of a different HA/NA type (e.g., A/Puerto Rico/8/1934 (H1N1)). Protection observed in such a challenge model would suggest a broadening of antigen-specific response to include antigens common to both strains, such as the stalk region of the HA protein. To confirm such broadening, a study of the B cell receptor (BCR) and T cell receptor (TCR) sequences is conducted.

### Outcomes and Alternative Approaches

As mentioned previously, increasing numbers of literature reports cite combinations of various TLR agonists that are able to synergistically increase the magnitude of vaccine-mediated immunity and change the type of downstream adaptive immune response generated thereby enhancing the efficacy of these vaccines. An adjuvant combination for influenza virus challenge protection is described herein.

### Example 3

### Influenza Hemagglutinin (HA) as a Vaccine Antigen

Strategies to boost broadly neutralizing stalk antibodies include: 1) focus on headless HAs, with the removal of the entire head domain to make the stalk domain more “available” and thus induce antibody responses against the stalk domain, or 2) use chimeric HAs consisting of the stalk domain from H1, H3 or influenza B viruses in combination.

It is known that immunization with one antigen blocks robust immune responses to a second, similar antigen (“original antigenic sin”). That is important for infectious diseases where there are repeated infections (influenza), or antigenic evolution (HIV, malaria). For influenza, major neutralizing antibodies made against the head region of the viral hemagglutinin (HA). Different viral strains have different HA head regions, that cross-react weakly with antibodies, but inhibit the response to new epitopes). For HIV, mutated epitopes on the virus do not stimulate antibodies or T cells because of epitope suppression

Mechanisms of original antigenic sin in vaccines may be due to epitope exclusion (pre-existing antibodies, especially mucosal IgA, shield the vaccine from all antigen presenting cells (APCs); dendritic cell access (memory B cells internalize the new vaccine, with reduced DC activation and T cell immunization); and/or T cell competition (memory B cells are activated, consuming cytokines, co-factors, and trapping T cells that could react with antigen loaded DCs

To overcome original antigenic sin in vaccines, dosage may be increased (e.g., a massive vaccine dose (patients over 60 receive 3× dose of influenza vaccine)); encapsulation (put the vaccine in an emulsion or liposome that preferentially delivers the vaccine to dendritic cells (Shingrix, varicella vaccine for shingles)); and/or dendritic cell activators (TLR agonists may increase the numbers diversity of activated T cells against the vaccine antigens).

To study original antigenic sine in mouse models, the following may be used: hapten-protein conjugates (a hapten is a small molecule like Flourescein or DNP that can be coupled to a protein antigen like ovalbumin and KLS); or pre-immunization with the unconjugated protein antigen inhibits antibody responses to immunization with the hapten-protein conjugate. For influenza in these models, hyper-immunize with one protein, such as influenza HA, for one viral strain, boost with a partially cross-reactive HA from another strain, then analyze B and T cell immune responses to the second HA, including epitopes recognized, clonal diversity by nexgen RNA sequencing, and neutralizing capacity, and then correlate with in vivo protection.

Shingrix is recombinant VSV glycoprotein E, nonophosphoryl lipid A from Salmonella, and QS-21 saponin molecule in a liposomal formulation made from dioleoyl phosphatidylcholine and cholesterol in buffered saline, which is reconstituted at time of use. To make an influenza vaccine analogous to Shingrix, the vaccine has a protein antigen, two adjuvants in a liposomal formulation.

### Example 4

The effectiveness of the annual influenza vaccine is still rated 10-60% because of antigenic drift of influenza virus. Synthetic TLR4 and TLR7 agonists (1Z105 and 1V270) enhanced Th2- and Th1-mediated anti-hemagglutinin antibody production, respectively. The combination with 1Z105 and 1V270 promoted the balanced Th1/Th2 immunity to protect against influenza virus infection. To enhance the adjuvant efficacy, a structure activity relationship study was conducted on 1Z105 and 2B182C was identified; a derivative with higher potency in vitro. In an in vivo vaccination study using the model antigen ovalbumin, 2B182C induced higher serum IgG1 levels and additively enhance the release of antigen-specific IgG2a induced by 1V270. Furthermore, the liposomal formulation of 2B182C and 1V270 reduced cytotoxicity and reactogenicity and maintained the activity to enhance both Th1- and Th2-mediated antibody production. In an in vitro vaccination study using inactivated A/California/04/2009 (H1N1) (pdm09) as antigen, the liposomal combination adjuvant increased the populations of T follicular helper cells, germinal center B cells and antibody secreting plasma cells. Next generation sequence analyses of B and T lymphocytes in the draining inguinal lymph nodes showed that the combined adjuvants increased B cell clonotypes of immunoglobulin heavy chain (IGH) genes, shared B cell clones and TCR clonalities. These findings suggested that the combination contributed to enhance antigen specific Th1 immune response. Finally, the vaccine with the combination adjuvants protected against lethal homologous virus challenge with less lung damage.

### Methods

### Mouse

Female 6-8 week-old BALB/c mice were purchased from Jackson laboratory (Bar Harbor, Mass.). The animal experiments using ovalbumin, or inactivated influenza virus as antigens which were not required a live virus challenge were performed at University of California San Diego Animal Facility. The influenza challenge study was performed by the Animal Research Center of Utah State University using female 6 week-old BALB/c mice (Charles River Laboratories, Wilmington, Mass.). All Animal experiments received prior approval by the Institutional Animal Care and Use Committee (IACUC) for UC San Diego or Utah State University.

### Cells and Reagents

TLR4/NF-kB reporter cell lines HEK-Blue™ humanTLR4 and HEK-Blue™ murineTLR4 cells were purchased from InvivoGen (Catalog numbers, San Diego, Calif.). Mouse primary BMDCs were prepared from bone marrow cells harvested from femurs of C57BL/6 mice. BMDCs were treated with indicated compounds in RPMI supplemented with 10% FBS (Omega, Tarzana, Calif.) and penicillin/streptomycin (100 unit/mL/100 μg/mL, Thermo Fisher Scientific, Waltham, Mass.). Monophospholipid A (MPLA), AddaVax were purchased from InvivoGen (Catalog numbers San Diego, Calif.). Inactivated Influenza A virus [A/California/04/2009 (H1N1) pdm09] (IIAV) were obtained from BEI resources (#NR-49450, Manassas, Va.). TLR7 agonist 1V270, TLR4 agonists 1Z105 and it derivatives including 2B182C were synthesized. Liposomal formulation of 1V270 (20 μM), 2B182C (4 mM) and 1V270+2B182C (20 μM+4 mM) was performed y Innimune corp. (Missoula, Mont.).

### TLR4/NF-κB Reporter Cell Assay

TLR4/NF-κB activation was assessed using HEK-Blue™ hTLR4 and HEK-Blue™ mTLR4 (InvivoGen). The cells were treated with 1Z105 and 2B182C (2-fold serial dilution starting from 10 μM) for 20h. NF-κB inducible secreted embryonic alkaline phosphatase (SEAP) protein in the culture supernatant was measured according to manufacturer's protocol.

### Evaluation of Antibody Production In Vivo

BALB/c mice were intramuscularly (i.m.) immunized with IAV (10 μg/injection) plus indicated adjuvants in gastrocnemius of hind legs on days 0 and 21. Detailed concentrations of adjuvants and the number of animals in each treatment are described in each figure legends. Sera were collected on day 28 and evaluated for antigen-specific antibodies (anti-HA IgG1, anti-NA IgG1, anti-HA IgG2a, anti-NA IgG2a, anti-HA IgM and anti-NA IgM). ELISA for these antibodies were performed as previously described (Ref). For studies with DMSO formulation, 10% DMSO was used as vehicle. In the experiments using the liposomal-formulated adjuvant. 1,2-dioleoyl-sn-glycero-3-phosphocholine and cholesterol (DOPC/Chol, control liposomes) was used as vehicle.

### NGS Assay for BCR and TCR Repertoire

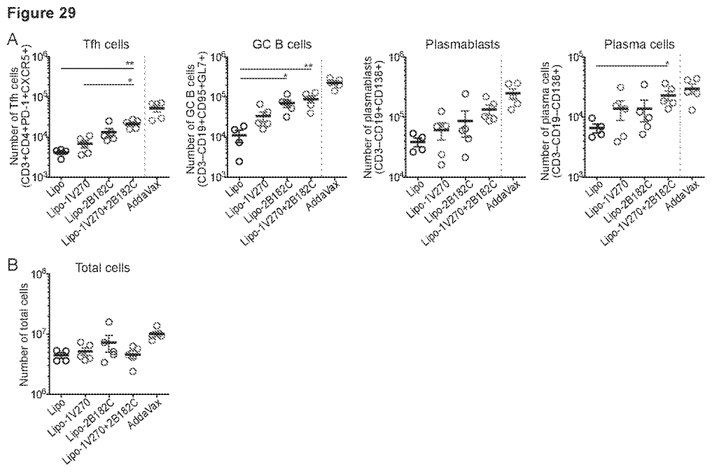

Immunization protocol was shown in FIG. 28A. Briefly, mice were sacrificed on day 28 and inguinal lymph nodes were harvested. Total RNA was extracted from lymphocytes (bulk) using RNeasy Mini Kit (Qiagen, Hilden, Germany) and the quality of RNA was confirmed by Agilent 4200 TapeStation (Agilent, Santa Clara, Calif.). Next-generation sequencing was performed with unbiased TCR repertoire analysis technology (Repertoire Genesis Inc., Osaka, Japan).

Evaluation for Protection from Lethal Influenza Virus Challenge

BALB/c mice were i.m. vaccinated with formulated 1V270 and 2B182C with IIAV (3 ug/injection) on day 0 and intranasally infected with homologous or heterologous influenza A virus, A/California/04/2009 (pdmH1N1) and A/Victoria/3/75 (H3N2) on day 21, respectively. The immunization dose of IIAV; 3 μg/injection that protect 30-50% of animal from the challenge with homologous virus was determined in the preliminary experiment. For influenza virus challenge, groups of mice were anesthetized by intraperitoneal injection of ketamine/xylazine (50 mg/kg/5 mg/kg) prior to intranasal challenge with 1×10⁵ (3×LD₅₀) cell culture infectious doses (CCID₅₀) of influenza A/California/04/2009 (H1N1pdm) virus per mouse; 5×10² (3×LD₅₀) CCID₅₀ of influenza A/Victoria/3/75 (H3N2) virus per mouse in a 90-μL suspension. All mice were administered virus challenge on study day 21. Influenza virus (H1N1pdm), strain designation 175190, was received from Dr. Elena Govorkova (Department of Infectious Diseases. St. Jude Children's jemResearch Hospital, Memphis Tenn.). The virus was adapted to replication in the lungs of BALB/c mice by 9 sequential passages in mice. Virus was plaque purified in Madin-Darby Canine Kidney (MDCK) cells and a virus stock was prepared by growth in embryonated chicken eggs and then MDCK cells. Influenza A/Victoria/3/75 (H3N2) virus was obtained from the American Type Culture Collection (Manassas, Va.). The virus was not lethal to mice initially, but became lethal after 7 serial passages in the lungs of infected animals. Following mouse-adaptation a virus stock was prepared by growth in MDCK cells.

### Determination of Lung Virus Titers and Lung Inflammation

Six days after virus challenge, the bronchioalveolar lavage (BAL) procedure was performed immediately after blood collection and was completed within 5 to 10 min of each animal's death. A volume of 0.75 mL of phosphate buffered saline (PBS) was slowly delivered into the lung through the tracheal tube. Immediately after delivery the fluid was slowly withdrawn by gentle suction and the samples were stored at −80° C. The procedure was repeated a total of three times and lavage fluids from each mouse were pooled. To determine lung virus titers, BAL samples were centrifuged at 2000 g for 5 minutes. Varying 10-fold dilutions of BAL supernatants were assayed in triplicate for infectious virus in MDCK cells, with virus titers calculated. For determination of lung cytokine levels, a sample (200 μL) from each lung lavage was tested for MCP-1 and IL-6 using a chemiluminescent multiplex ELISA-based assay according to the manufacturer's instructions (Quansys Biosciences Q-Plex™ Array, Logan, Utah).

### Hemagglutination Inhibition Titers

For hemagglutination inhibition (HI) titers, sera were pre-treated with receptor-destroying enzyme II (RDE; Vibrio cholerae neuraminidase; YCC-340; Accurate Chemical and Scientific, Westbury, N.Y.) to remove non-specific inhibitors by diluting one part serum with three parts enzyme and incubating at 37° C. for 18 h. RDE was subsequently inactivated by heating at 56° C. for 45 min. Serum samples were diluted in PBS in 96-well round-bottom microtiter plates (Fisher Scientific, Pittsburgh, Pa.). Following dilution of serum, 8 HA units/well of influenza A/CA/04/2009 (H1N1pdm) or influenza A/Victoria/3/75 (H3N2) viruses plus turkey red blood cells (Lampire Biological Laboratories, Pipersville, Pa.) were added (50 μL per well), mixed briefly, and incubated for 60 min at room temperature. The HI titers of serum samples are indicated as the reciprocal of the highest serum dilution at which hemagglutination was completely inhibited.

### Virus Neutralization Titers

For anti-influenza virus neutralizing antibody assay, MDCK cells were seeded in 96-well plates at 1×10⁴ cells per well in MEM containing 5% FBS (Hyclone, Logan, Utah) 24 h prior to use. Serial 2-fold dilutions of serum samples were prepared in serum-free media, containing 10 units/mL trypsin and 1 μg/mL EDTA, starting at 1:32 dilution and ending at 1:4096. Each serum dilution was mixed 1:1 (0.1 mL) with serum-free media (containing trypsin and EDTA) containing approximately 100 CCID50/well H1N1pdm or influenza A/Victoria/3/75 (H3N2) virus. After incubation at room temperature for 1 h, the serum-influenza virus mixture (0.2 mL) was transferred to a well containing MDCK cells and incubated for 3 days. Anti-influenza virus neutralizing antibodies were measured as cytopathic effect (CPE) inhibition. CPE was scored from duplicate samples by examining the MDCK cell monolayers under a light microscope on day 3 post-infection.

### Statistical Analyses

Data obtained in in vivo studies are presented as means with standard error of mean (SEM) and in vitro data are indicated as means with standard deviation (SD). For in vitro data, a two tailed Welch's t test was used to compare two groups. For antigen specific antibodies, flow cytometry analysis for immune cell populations, BCR-seq, TCR-seq, lung virus titers, HI endpoint titers, and VN endpoint titers, Kruskal-Wallis tests with Dunn's post hoc test were applied. Correlations between lung virus titers and cytokine/chemokine levels were analyzed using a Spearman rank correlation test. For body weight, area under the curve was calculated for each mouse and one-way ANOVA was used for statistical analysis. The log rank (Mantel-Cox) test was used to test for a significant difference between Kaplan-Meier survival curves. Prism 5 software (GraphPad Software, San Diego, Calif.) was used. A P value less than 0.05 was considered statistically significant.

TABLE 8
Reagents used in ELISA for hIL-8, mIL-12 and mIL-6

| Reagents | Dilution factor | Source | Catalog # |
|---|---|---|---|
| Capture antibodies | | | |
| Purified mouse anti-human IL-8 | 250 | BD Biosciences | 554716 |
| Purified rat anti-mouse IL-12 | 200 | BD Biosciences | 551219 |
| Purified rat anti-mouse IL-6 | 100 | BD Biosciences | 554400 |
| Detecting antibodies | | | |
| Biotin mouse anti-human IL-8 | 1000 | BD Biosciences | 554718 |
| Biotin rat anti-mouse IL-12 | 1000 | BD Biosciences | 554476 |
| Biotin rat anti-mouse IL-6 | 1000 | BD Biosciences | 554402 |
| Other reagents | | | |
| Streptavidin, HRP | 1000 | Thermo Fisher | 43-4323 |
| | | Scientific | |
| KPL SureBlue ℠ TMB | | Seracare | 5120-0077 |
| Peroxidase Substrate | | | |

TABLE 9
Reagents used in ELSA for hIL-8, mIL-12 and mIL-6

| Antibodies (clone) | Dilution factor | Source | Catalog # |
|---|---|---|---|
| Anti-CD86, APC/Cy7 (GL1) | 200 | BioLegend | 105030 |
| Anti-CD40, PE (1C10) | 200 | eBioscience | 12-0401 |
| Anti-CD3, BV510 (145-2C11) | 200 | BD Biosciences | 563024 |
| Anit-CD19, FITC (1D3) | 500 | BD Biosciences | 553785 |
| Anti-CD4, e450 (RM4-5) | 1500 | eBioscience | 48-0042 |
| Anti-CD95, PE/Cy7 (Jo2) | 500 | BD Biosciences | 557653 |
| Anti-CD138, APC (281-2) | 200 | BD Biosciences | 558626 |
| Anti-GL7, Pacific Blue (GL7) | 350 | BioLegend | 144614 |
| Anti-PD-1, APC (J43) | 150 | BD Biosciences | 562671 |
| Anti-CXCR5, Biotin (2G8) | 50 | BD Biosciences | 551960 |
| Anti-CD16/32 (FcR) | 300 | BD Biosciences | 553142 |
| Streptavidin PE | 500 | BD Biosciences | 554061 |
| Propidium Iodide Staining | 400 | BD Biosciences | 556463 |
| Solution | | | |
| Stain buffer | | BD Biosciences | 554657 |

TABLE 10
Reagents used in ELISA for IgGs

| Reagents | | Source | Catalog # |
|---|---|---|---|
| Proteins for coating | Concentrations | | |
| Influenza A H1N1 | 100 ng/mL | Sino | 11055- |
| (A/California/04/2009) Hemagglutinin/ | | Biological | V081-1 |
| HA Protein (Hs Tag) | | | |
| Influenza A H1N1 (A/Puerto | 100 ng/mL | Sino | 11684- |
| Rico/8/1934) Hemagglutinin/HA | | Biological | V08B |
| Protein (His Tag) | | | |
| Influenza A H3N2 (A/Victoria/3/1975) | 100 ng/mL | Sino | 40396- |
| Hemagglutinin/HA1 Protein (His Tag) | | Biological | V08H1 |
| Influenza A H7N7 | 100 ng/mL | Sino | 11082- |
| (A/Netherlands/219/2003) | | Biological | V08B |
| Hernaggiutinin/HA Protein (His Tag) | | | |
| Influenza A H11N9 | 100 ng/mL | Sino | 11704- |
| (A/mallard/Alberta/294/1977) | | Biological | V08H |
| Hernagglutinin/HA Protein (His Tag) | | | |
| Influenza A H12N5 (A/green-winged | 100 ng/mL | Sino | 11718- |
| teal/ALB/199/1991) Hemaggiutinin/ | | Biological | V08H |
| HA Protein (His Tag) | | | |
| Influenza A H1N1 | 100 ng/mL | Sino | 11058- |
| (A/California/04/2009) Neuraminidase/ | | Biological | V07B |
| NA (Fc Tag) | | | |
| Influenza A H5N1 (A/Thailand/1 (KAN- | 100 ng/mL | Sino | 40064- |
| 1)/2004) Neuraminidase/NA (His | | Biological | V07H |
| Tag) | | | |
| Influenza A H3N2 (A/Babol/36/2005) | 100 ng/mL | Sino | 40017- |
| Neuraminidase/NA (His Tag) | | Biological | V07H |
| Influenza A H10N8 | 100 ng/mL | Sine | 40352- |
| (A/duck/Guangdong/E1/2012) | | Biological | V07B |
| Neuraminidase/NA Protein (Hs Tag) | | | |
| Influenza A H7N7 | 100 ng/mL | Sino | 40202- |
| (A/Netherlands/219/2003) | | Biological | V07H |
| Neuraminidase/NA Protein (His Tag) | | | |
| Antibodies | Dilution factor | | |
| IgGl-AP goat anti-mouse | 2000 | Southern | 1070-04 |
| | | Biotech | |
| IgG2a-AP goat anti-mouse | 2000 | Southern | 1080-04 |
| | | Biotech | |
| IgG-AP goat anti-mouse | 2000 | Southern | 1030-04 |
| | | Biotech | |
| p-Nitrophenyl Phosphate tablets | | Sigma | N2770 |
| (pNPP) | | | |

### Results

### Structure Activity Relationship Study of 1Z105 Yielded 2B182C

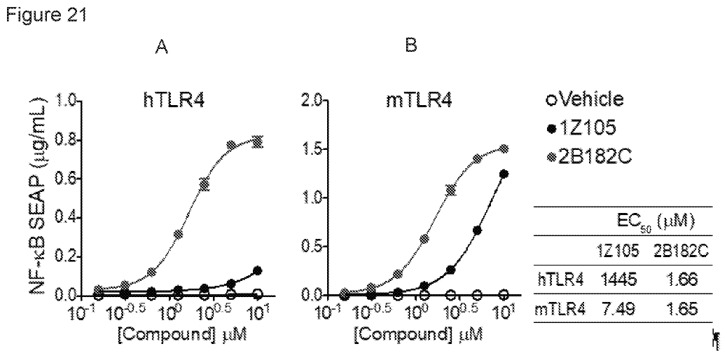

To improve the potency to the small molecule pyrimidoindole TLR4 ligand, 1Z105, the structure activity relationship analysis was performed (Chemists will fill out). A total of 56 compounds were synthesized, and screened by human and murine HEK TLR4 reporter cells (HEK-Blue mTLR4 and hTLR4, respectively). Among those SAR compounds, 23182C was discovered as a derivative with higher TLR4 stimulatory potency in both murine and human reporter cells. The EC₅₀ of 26182C was examined using HEK TLR4 reporter cells and compared to the EC50 of 1Z105 (FIG. 21B). EC50 of 2B182C in murine and human TLR4 reporter cells was increased by 5.8 fold and 870-fold, respectively, in comparison with EC50 of 1Z105. These data indicate that SAR study successfully yielded a derivative exhibiting higher TLR4 stimulatory potency, notably human TLR4 potency.

### TLR4 Agonist 2B182c Enhanced Antigen Specific IgG1 Production

TLR4 agonist 1Z105 induced Th2-mediated IgG1 production and TLR7 agonist 1V270 enhanced Th1 cellular immunity against influenza virus (Goff at al., J. Virol., 89:3221 (2015); Goff et al., J. Virol., 91:001050 (2017)). It was hypothesized that by combining with 1V270, the efficacy of the TLR4 agonist 2B182C as an influenza vaccine adjuvant could be improved. Therefore, it was examined whether 2B182C with 1V270 improved the adjuvanticity in vivo compared to the combo adjuvants with 1Z105 plus 1V270.

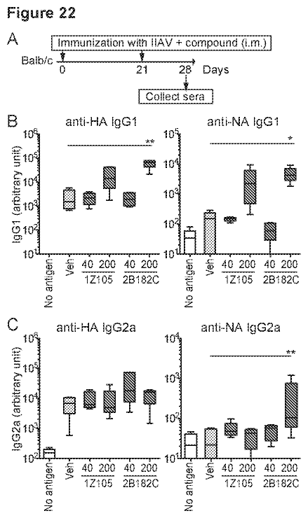

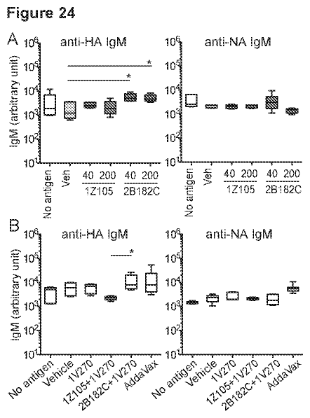

To develop the effective combined vaccine adjuvants, the potency of 1Z105 and 2B182C, and optimal dose as a single agent, were compared using inactivated Influenza A virus [A/California/04/2009 (H1N1) pdm09] (IIAV) as an antigen. BALB/c mice were immunized on days 0 and 21 with IIAV mixed with the TLR4 agonists, 1Z105 or 2B182C, were bled on day 28 (FIG. 22A). Sera were evaluated by ELISA for antibodies (IgM, IgG1 and IgG2a) against two glycoproteins on the surface of the virus, hemagglutinin (HA) and neuraminidase (NA). 1Z105 and 2B182C were dissolved in DMSO and the final concentration of DMSO was 10%. The results showed that 2B182C with higher dose as 200 nmol/injection significantly increased IgG1 antibody against both HA and NA (FIG. 22B). Interestingly, 2B182C, but not 1Z105, enhanced anti-NA specific IgG2a (FIG. 12C). Anti-HA IgM level was only slightly increased by 2B182C (FIG. 24A).

Combination with 2B182C and TLR7 Agonist 1V270 Increased Both Antigen Specific IgG1 and IgG2a

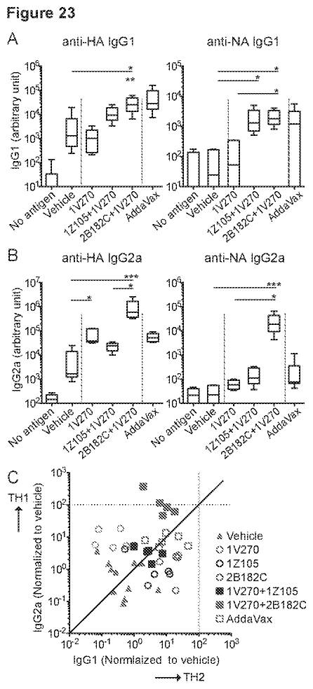

Next the co-adjuvant effects of these TLR4 agonists on antibody production was analyzed when combined with TLR7 agonist 1V270 at a dose of 1 nmol/injection, which was reported to induce IgG2a production enhancing Th1 immune responses (Goff at al., 2017). The results indicated that while 1V270 alone induced only anti-HA IgG2a production, when combined with 2B182C, IgG1 and IgG2a antibodies against both HA and NA were significantly induced. This suggests that these compounds may work in an additive manner (FIGS. 23A and 23B). On the other hand, 1Z105 failed to enhance IgG2a production induced by 1V270. Animals in 1V270+2B182C-group produced higher amount of both IgG1 and IgG2a and the immune balance was inclined toward Th1-mediated IgG2a production, suggesting that the treatment contribute to enhance Th1 immune responses (FIG. 23C). The combination with 1V270 and 2B182C showed moderate effect on anti-HA IgM production (FIG. 24B).

Collectively, the combination of 200 nmol/injection 2B182C plus 1 nmol/injection 1V270 induced highest quantity of antigen specific IgG1 and IgG2a and Th1-skewing immune responses, which are desirable for protection in the influenza virus infection. Thus, this combination was selected for the next formulation.

### Liposomal Formulation Upgraded 2B182C Reducing Cytotoxicity

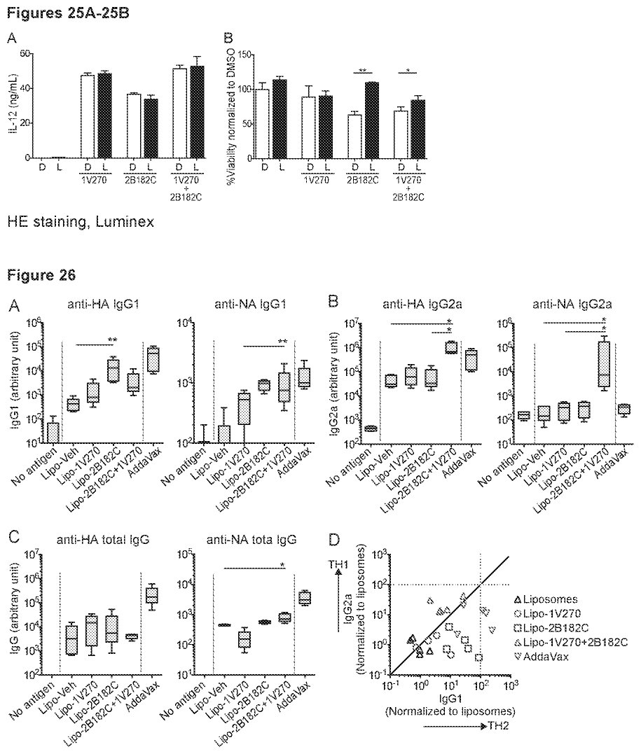

Given the results above, a 1V270/2B182C ratio (TLR4/TLR7) of 1/200 [1 nmol/injection (20 μM) 1V270 and 200 nmol/injection (4 mM) 2B182C] was used. In order to avoid unwanted cytotoxicity and reactogenicity while maintaining response to vaccine, adjusting formulation of compounds may be important in the development of vaccine adjuvants. Therefore, 1V270 and 2B182c were formulated in liposomes by Inimmune Corp (Missoula, Mont.). The activity of the formulated compounds was tested in mouse primary BMDCs. These formulated compounds maintained similar levels of IL-12 secretion as DMSO-formulation compounds (FIG. 25A). Cytotoxicity induced by DMSO-2B182C or DMSO-1V270+2B182C were significantly improved by liposomal formulation. (FIG. 25B). Histological analysis by H&E staining of muscles in the injected sites is shown in FIG. 25C. Multiplex cytokine/chemokine analysis of sera after administration of the compounds is shown in FIG. 25D.

### Liposomal 1V270 and 2B182C Synergistically Enhanced Anti-HA and Anti-NA IgG1 and IgG2a Production

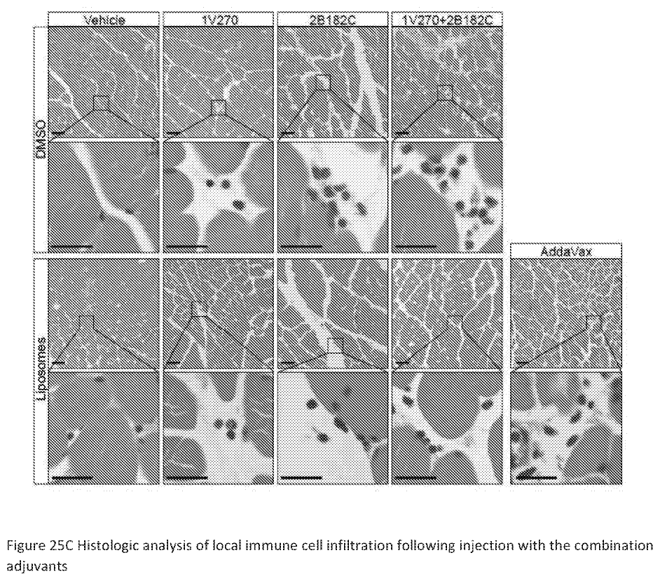

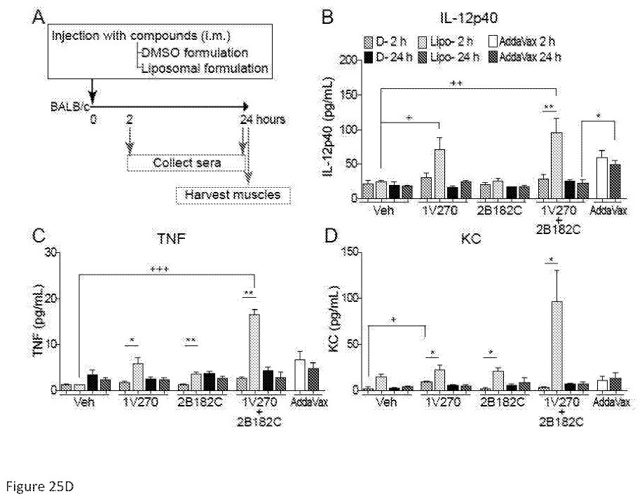

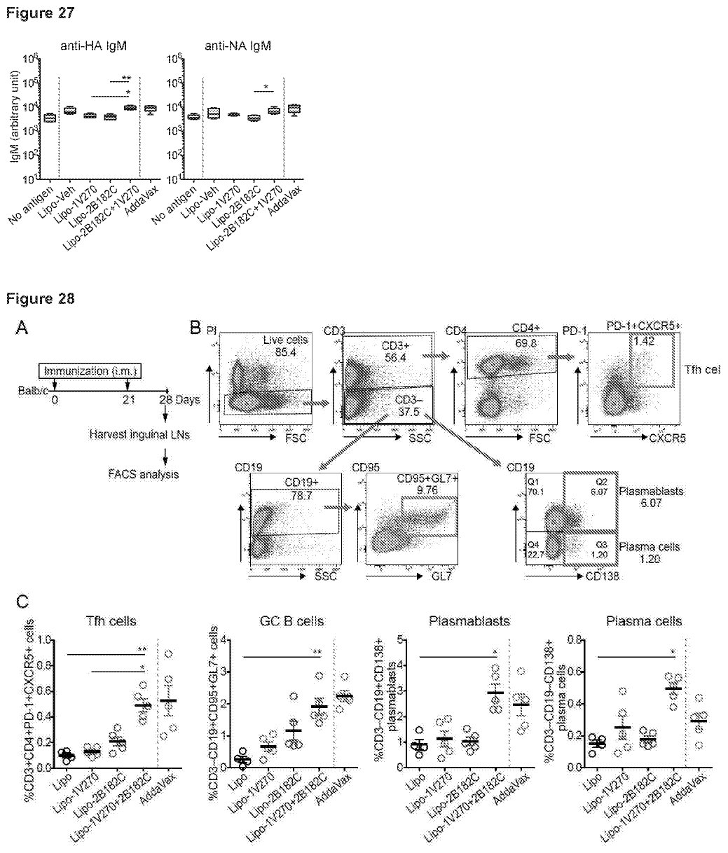

The adjuvanticity of the compounds in vivo was evaluated using prime-boost regimen as described in FIG. 22A. Sera harvested on day 28 were assessed for antigen specific antibodies by ELISA. The results indicated that lipo-2B182C induced higher level of IgG1, which was consistent with DMSO-2B182C (FIG. 26A). Unlike DMSO-1V270, lipo-1V270 alone did not promote IgG2a production (FIG. 26B). Despite these minimal effects on IgG2a by each agonist, when two adjuvants were combined, antigen specific antibody production was synergistically enhanced (FIG. 26B). On the other hand, total IgG levels induced by liposomal vehicle, 1V270, 2B182C and 1V270+2B182C, were comparable (FIG. 26C). Antigen specific IgM levels were not affected by any treatment (FIG. 27). Consistent with the trend observed with DMSO formulation, the liposomal combined adjuvants developed Th1-biased immune balance (FIG. 26D).

### Formulated 1V270 Plus 2B182C Enhanced Antibody Secretion Responses

To investigate whether the formulated adjuvants induces an activation of B cells promoting antigen specific antibody secretion, lymphocytes in inguinal lymph nodes were examined for Tfh cells, GC B cells, plasmablasts and plasma cells using flow cytometry. The immunization protocol described above was used and lymphocytes in the inguinal lymph nodes were harvested on day 28 and analyzed by flow cytometry (FIGS. 28A and 28B). As the results, the percentage of Tfh cells, which were identified as CD3+ CD4+ PD-1+ CXCR5+ cells, was greatly increased by lipo-1V270+2B182C (FIG. 28B and FIG. 29). Additionally, the combined adjuvants increased the percentage of GC B cells (CD3− CD19+ CD95+ GL7+). Increased plasmablasts and plasma cells were observed in mice vaccinated with lipo-1V270+2B182C. The results suggest that the combined adjuvants enhance GC reaction compared to a single agent.

Increased BCR Diversity and TCR Clonality by the Combo Adjuvant with 1V270 Plus 2B182C

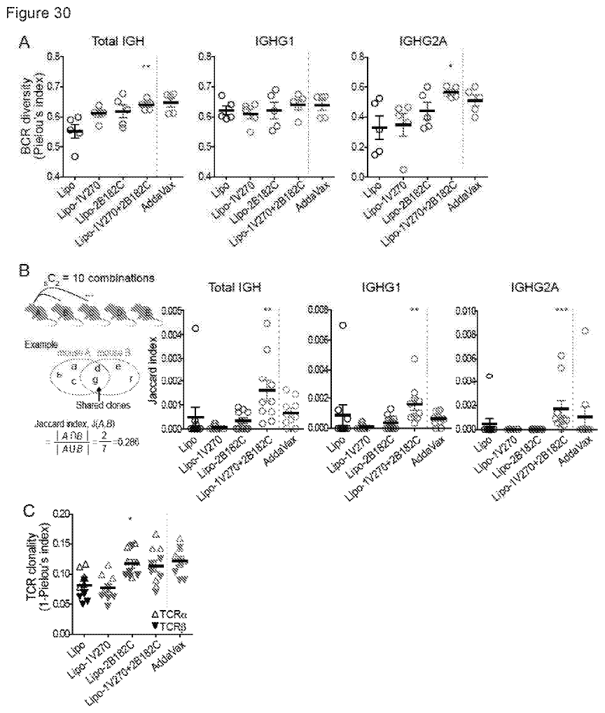

To examine whether the combined adjuvants affect the diversity of BCR, next generation sequencing analysis was performed for IGH genes (by Repertoire Genesis Inc, Osaka, Japan). The prime-boost IIAV model were used and lymphocytes in the inguinal lymph nodes were collected on day 28 (FIG. 30A). BCR sequence analyses showed that BCR diversity normalized to total reads indicated by Pielou's index was significantly increased by lipo-1V270+2B182C (FIG. 30A). Clonotypes of IgG genes were analyzed by similarity analysis, which compare IGH clones between two mice within the group to see if there is a shared clone and calculate Jaccard index: Jaccard index; J (A, B)=(A∩B)/(A∪B) (FIG. 30B). Jaccard indices for IGH. IGHG1 and IGHG2A were significantly increased by lipo-1V270+2B182C, indicating that clones shared between two mice within this group were increased. Furthermore, in the lipo-1V270+2B182c group, 6 clones (0.03%) were shared among three mice. These results suggest that the liposomal combined adjuvant increased BCR diversity in total IGH and IGHG2A. That is consistent to the higher IgG2a level following immunization of combined adjuvant. The common clones detected in the group immunized with the combined adjuvant might recognize dominant epitope(s) of the antigen. TCR sequencing was performed to see whether the formulated adjuvants contribute to increase f TCR clonality toward antigens. Expectedly, the combined adjuvants and lipo-2B182C increased clonalities of TCRα and TCRβ (FIG. 30C). Collectively, animals in lipo-1V270+2B182C showed higher BCR diversity and TCR clonality. This may support the data that Th1 response is enhanced by the combined adjuvants.

### Lipo-2B182C and Lipo-1V270+2B182C Protect Mice Against Homologous Influenza Virus.

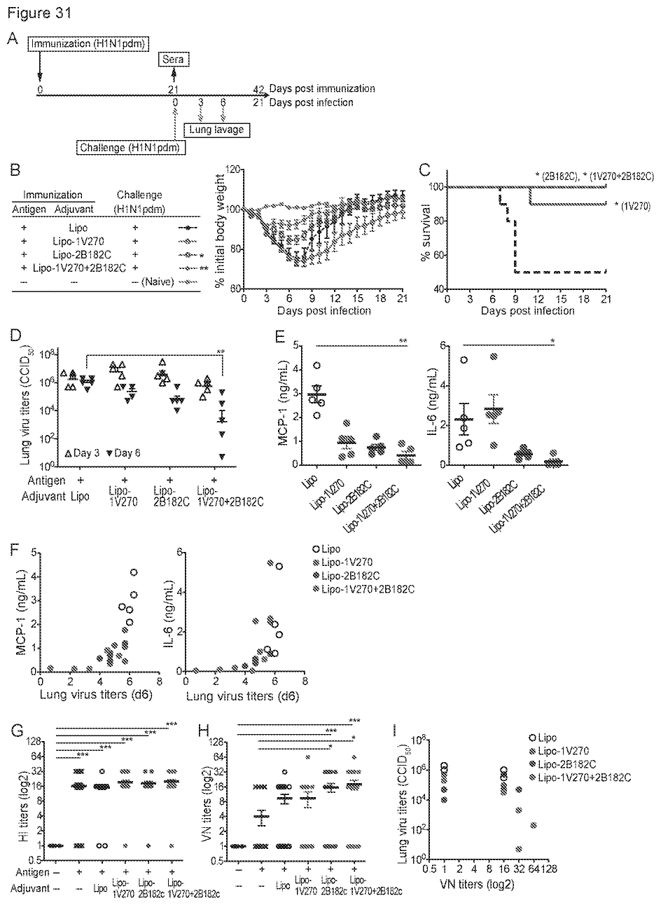

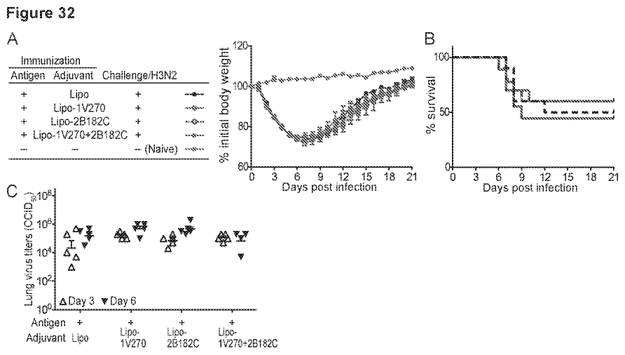

The combined adjuvant induced Th1 biased immune response accompanying diverse BCR and high clonality of TCR. To test whether this diversity could be an indication of an immune response against influenza virus, the formulated 1V270 and 2B182c were tested in the homologous and heterologous influenza virus challenge model. Balb/c mice vaccinated with IIAV plus liposomal 1V270, 2B182C or 1V270+2B182C were intranasally challenged with homologous (H1N1) influenza virus on day 21 post vaccination (single dose). Body weight and survival of mouse were monitored through additional 21 days (FIG. 31A). Lipo-2B182C and lipo-1V270+2B182C significantly suppressed body weight loss after viral infection (FIG. 31B). Furthermore, lipo-1V270 showed 90% protection, and lipo-2B182C and lipo-1V270+2B182C completely protected mice against homologous influenza virus (FIG. 31C). To evaluate if the survival of mice is correlated to viral titers in lung, bronchoalveolar lavage were performed for virus titers in lavage fluid. The results indicated that lipo-1V270+2B182C effectively suppressed virus titers in lungs on day 6 (FIG. 31D). In human, there is an upregulation of cytokine and chemokine in airway epithelial cells (e.g., MCP-1, IL-6, etc.) correlated with lethal lung injury and pneumonia (Gurczynski et al., Mucosal Immun., 12:518 (2019); Zhou et al., Nature, 499:500 (2013)). Therefore, we evaluated pro-inflammatory cytokine (IL-6) and chemokine (MCP-1) level in lung fluids using the Quansys multiplex ELISA. The results showed the combined liposomal adjuvants significantly suppressed both MCP-1 and IL-6 productions (FIG. 31E). The levels of pro-inflammatory cytokines were correlated to lung virus titers [MCP-1 (P<0.0001, Spearman r=0.83), IL-6 (P<0.0001, Spearman r=0.79) (FIG. 31F). This trend was further enhanced in lipo-1V270+2B182C group. These results suggested that the combined adjuvants reduced lung damage by inhibiting virus entry and proliferation after infection. To evaluate if the protection was related to the hemagglutination inhibition titers (HI) and virus neutralization titer (VN), sera were collected on day 21 post immunization and examined for HI and VN (FIG. 31A). The increased HI titers compared to non-immunized group were observed in 19 mice out of 20 mice in the lip-1V270, lipo-2B182C and lipo-1V270+2B182C (FIG. 31G). In addition, lipo-2B182c and lipo-1V270+2B182C induced significantly higher VN compared to liposomal control (FIG. 31H). VN titers were negatively correlated with lung virus titers (P<0.01, Spearman r=−0.59, FIG. 27I). Protection against heterologous Influenza virus A/Victora3/75 (H3N2) was evaluated using the same protocol as homologous challenge experiment (FIG. 31A). There was not significant difference in body weight loss, survival and lung virus titers in comparison to the liposomal control group (FIGS. 32A-C). Collectively, the formulated combined adjuvants showed significant protection against homologous H1N1 virus without adverse inflammatory effects, although it was insufficient for heterologous protection.

TABLE 11
Number of shared clones of total IgG genes in BCR-seq

| | | | | | |
|---|---|---|---|---|---|
| #clones | | Lipo- | Lipo- | Lipo- | |
| (%) | Vehicle | 1V270 | 2B1820 | 1V270 + 2B182C | AddaVax |

# clones (%)

| | | | | | |
|---|---|---|---|---|---|
| Not | 11418 | 14387 | 9157 | 18037 | 19019 |
| shared | (99.7) | (100.0) | (99.9) | (99.5) | (99.7) |
| 2 mice | 31 (0.27) | 4(0.03) | 10 (0.11) | 90 (0.50) | 51 (0.27) |
| 3 mice | 0 (0) | 0 (0) | 1(0.01) | 6 (0.03) | 0 (0) |
| 4 mice | 0 (0) | 0 (0) | 0 (0) | 0 (0) | 0 (0) |
| 5 mice | 0 (0) | 0 (0) | 0 (0) | 0 (0) | 0 (0) |

BALBIc mice were vaccinated on days 0 and 21 with 1IAV with formulated adjuvants.
Lymphocytes in the inguinal lympn nodes were harvested on day 28 for next generation sequencing for IGH genes.
Similarity analysis of IGH clonotype were performed.
Number of clones shared in 2, 3, 4 and 5 mice within a group and not shared were shown.
Six clones were shared between 3 mice in the combination group.

### Example 5

### Liposomal Co-Encapsulation of 1V270(TLR7 Ligand) and 2B182C(TLR4 Ligand) Broadens Antibody Epitopes

A universal vaccine for influenza virus infections requires the induction of antibodies that recognize broad epitopes of the major antigenic molecules, hemagglutinins (HA), and neuraminidase (NA). Thus, the epitope spreading and cross-reactivities of antibodies induced by the combined adjuvant (1V270 and 2B182C) were examined. BALB/c mice (n=5-10) were immunized with inactivated virus mixed with liposomal formulation of 1V270 (Lipo-1V270), 2B182C (Lipo-2B182C), co-encapsulated liposomal 1V270+2B182C [Lipo-(1V270+2B182C)], and add-mixed Lipo-1V270 and Lipo-2B182C in separate liposomes. Blank liposomes were used as a control and immunization was performed on day 0 (prime) and day 21 (boost) and sera were collected on day 28.

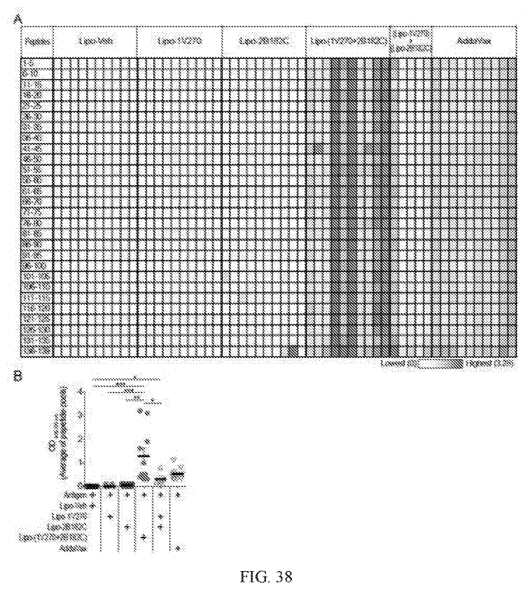

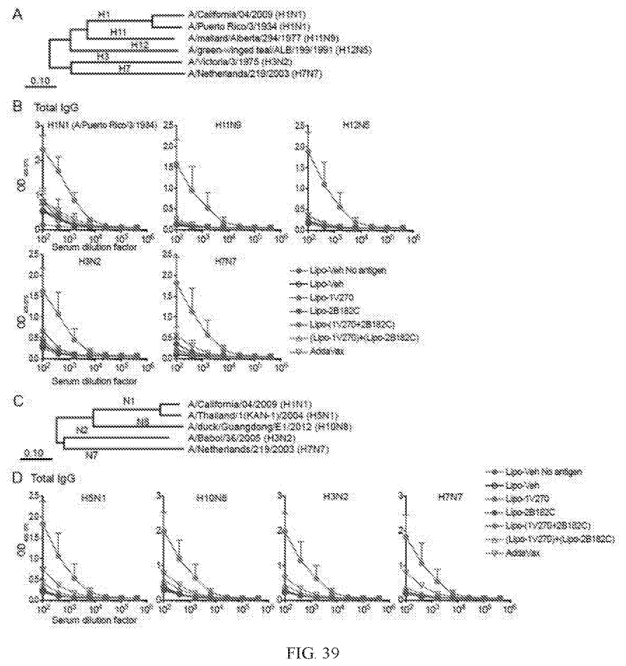

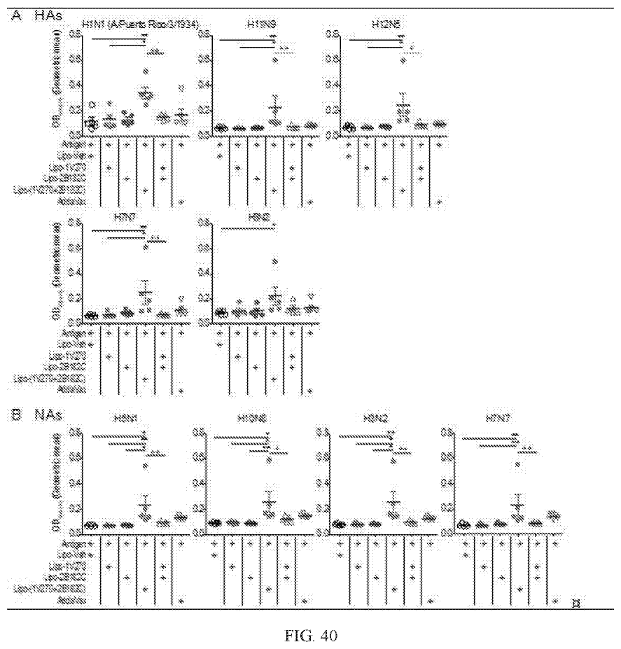

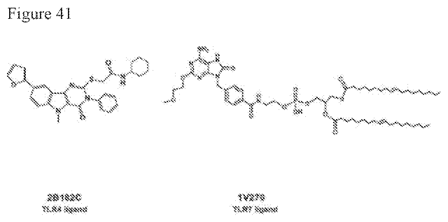

Epitope spreading was evaluated by HA peptide ELISA. Overlapping HA peptide array (139 peptides) of the Influenza A(H1N1)pdm09 virus was obtained from BEI Resources. Pooled peptides comprised of 5 consecutive peptides (total of 28 pools) were plated onto the ELISA plates. 1:200 diluted sera were tested for reactivity to each peptide pool by OD405-570. The OD of each serum was plotted on the heatmap (FIG. 38A), and the average OD of individual animals were compared. The sera from the mice vaccinated with co-encapsulated liposomal 1V270+2B182C [Lipo-(1V270+2B182C)] showed significantly higher OD compared to the liposomal formulation of single ligands or admix (FIG. 38B). These data indicate that Lipo-(1V270+2B182C) induced antibody responses recognizing a wide range of HA epitopes. To test whether the recognition of broad HA epitopes induced by Lipo-(1V270+2B182C) is associated with the cross-protection of different subtypes of influenza virus infection, we tested the cross-reactivity of antibodies against various subtypes of HA and NA by ELISA (FIGS. 39 and 40). Subtypes HAs and NAs that belong to different phylogenic distances. Geometric mean titer (GMT) of IgG from mice immunized with co-encapsulated Lipo-(1V270+2B182C) showed high reactivity not only with HAs from group 1 (H1, H11. H12) but also with HAs in group 2 (H3 and H7) in comparison to liposomal single ligand, or add-mixed two separate liposomes. Broadened reactivities were also observed to different subtypes of NA. In summary, antibodies produced in the animals vaccinated with IIAV plus Lipo-(1V270+2B182C) were highly cross-reactive to different subtypes of HA and NA.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to enhance an immune response in a mammal, comprising administering to a mammal in need thereof a composition comprising liposomes comprising an effective amount of a TLR4 agonist and a TLR7 agonist.

2. The method of claim 1 wherein the TLR4 agonist and a TLR7 agonist are administered simultaneously.

3. The method of claim 1 or 2 wherein the TLR4 agonist has formula (II): 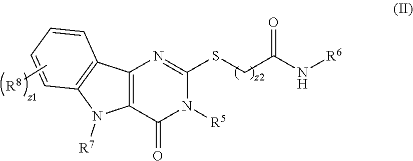
wherein zI is an integer from 0 to 4, wherein z2 is an integer from 0 to 5, wherein R⁵ is substituted or unsubstituted cycloalkyl, substituted or unsubstituted heterocycloalkyl, substituted or unsubstituted aryl, or substituted or unsubstituted heteroaryl, wherein R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R⁷ is hydrogen, or substituted or unsubstituted alkyl, and wherein each R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

4. The method of any one of claims 1 to 3 wherein the TLR7 agonist has formula (I): 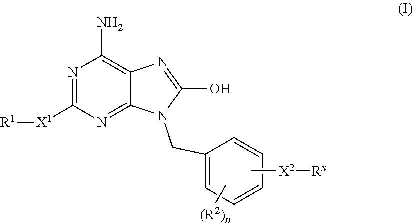
wherein X¹ is —O—, —S—, or —NR^{c}—;
R¹ is hydrogen, (C₁-C₁₀)alkyl, substituted (C₁-C₁₀)alkyl, C₅₋₁₀aryl, or substituted C₆₋₁₀aryl, C₅₋₉heterocyclic, substituted C₅₋₉heterocyclic;
R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl),
substituted —C(O)O(C₁-C₆)alkyl, —NR^{a}R^{b}, —C(O)NR^{a}R^{b} (carbamoyl), halo, nitro, or cyano, or R² is absent:
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4;
X² is a bond or a linking group; and
in one embodiment, R^{x} is a phospholipid comprising one or two carboxylic esters;
or a tautomer thereof;
or a pharmaceutically acceptable salt or solvate thereof

5. The method of any one of claims 1 to 4 wherein the liposomes comprise PC, DOPC, or DSPC.

6. The method of any one of claims 1 to 4 wherein the liposomes comprise cholesterol.

7. The method of any one of claims 1 to 6 further comprising administering one or more immunogens.

8. The method of claim 7 wherein the immunogen is a microbial immunogen.

9. The method of claim 8 wherein the microbe is a virus or a bacteria.

10. The method of any one of claims 7 to 9 wherein the liposomes comprise the one or more immunogens.

11. The method of any one of claims 1 to 10 wherein the mammal is a human.

12. The method of any one of claims 1 to 11 wherein the amount of the TLR7 agonist is about 0.01 to 100 nmol, about 0.1 to 10 nmol, or about 100 nmol to about 1000 nmol.

13. The method of any one of claims 1 to 12 wherein the amount of the TLR4 agonist is about 2 to 20 umol, about 20 nmol to 2 umol, or about 2 umol to about 100 umol.

14. The method of any one of claims 1 to 13 wherein the ratio of TLR7 to TLR4 agonist is about 1:10, 1:100, 1:200, 5:20, 5:100, or 5:200.

15. The method of any one of claims 1 to 13 wherein the composition is injected, intramuscularly administered, intranasally administered or intravenously administered.

16. The method of any one of claims 1 to 15 wherein the liposomes comprise DOPC and cholesterol.

17. A pharmaceutical formulation comprising liposomes, a TLR4 agonist and a TLR7 agonist.

18. The formulation of claim 17 wherein the liposome comprises 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; 1,2-distearoyl-sn-glycero-3-phosphocholine (DSPC), 1,2-dioleoyl-sn-glycero-3-phosphoethanolamine (DOPE), cholesterol, or a mixture thereof.

19. The formulation of claim 17 wherein the liposome comprises DOPC, cholesterol or combinations thereof.

20. The formulation of any one of claims 17 to 19 wherein the amount of the TLR7 agonist is about 0.01 to 100 nmol, about 0.1 to 10 nmol, or about 100 nmol to about 1000 nmol.

21. The formulation of any one of claims 17 to 20 wherein the amount of the TLR4 agonist is about 2 nmol to 20 umol, about 20 nmol to 2 umol, or about 2 umol to about 100 umol.

22. The formulation of any one of claims 17 to 21 wherein the ratio of TLR7 to TLR4 agonist is about 1:10, 1:100, 1:200, 5:20, 5:100, or 5:200.

23. The formulation of any one of claims 17 to 22 wherein the TLR7 agonist comprises a compound of Formula (I): 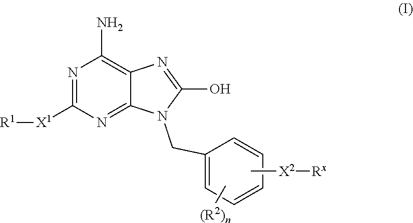
wherein X¹ is —O—, —S—, or —NR^{c}—;
R¹ is hydrogen, (C₁-C₁₀)alkyl, substituted (C₁-C₁₀)alkyl, C₅₋₁₀aryl, or substituted C₆₋₁₀aryl, C₅₋₉heterocyclic, substituted C₅₋₉heterocyclic;
R^{c} is hydrogen, C₁₋₁₀alkyl, or substituted C₁₋₁₀alkyl: or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl),
substituted —C(O)O(C₁-C₆)alkyl, —NR^{a}R^{b}, —C(O)NR^{a}R^{b}(carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆ alkyl, hydroxyC₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4;
X2 is a bond or a linking group; and
R³ is a phospholipid comprising one or two carboxylic esters;
or a tautomer thereof;
or a pharmaceutically acceptable salt or solvate thereof.

24. The formulation of claim 23 wherein R³ in formula (I) comprises 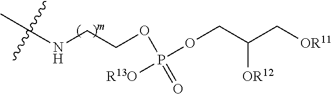
wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof.

25. The formulation of claim 23 or 24 wherein m is 1 or wherein R¹¹ and R¹² are each oleoyl groups.

26. The formulation of any one of claims 23 to 25 wherein the phospholipid of R³ comprises two carboxylic esters and each carboxylic ester includes one, two, three or four sites of unsaturation, epoxidation, hydroxylation, or a combination thereof.

27. The formulation of any one of claims 23 to 26 wherein the phospholipid of R³ comprises two carboxylic esters and the carboxylic esters of are the same or different.

28. The formulation of claim 27 wherein each carboxylic ester of the phospholipid is a C17 carboxylic ester with a site of unsaturation at C8-C9.

29. The formulation of claim 27 wherein each carboxylic ester of the phospholipid is a C18 carboxylic ester with a site of unsaturation at C9-C10.

30. The formulation of any one of claims 23 to 29 wherein X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.

31. The formulation of any one of claims 23 to 30 wherein R³ comprises dioleoylphosphatidyl ethanolamine (DOPE).

32. The formulation of any one of claims 23 to 31 wherein R³ is 1,2-dioleoyl-sn-glycero-3-phospho ethanolamine and X² is C(O).

33. The formulation of any one of claims 23 to 32 wherein X¹ is oxygen.

34. The formulation of any one of claims 23 to 33 wherein X¹ is O, R¹ is C₁₋₄alkoxy-ethyl, n is O, X² is carbonyl, and R³ is 1,2-dioleoylphosphatidyl ethanolamine (DOPE).

35. The formulation of any one of claims 23 to 33 wherein the compound of Formula (I) is:

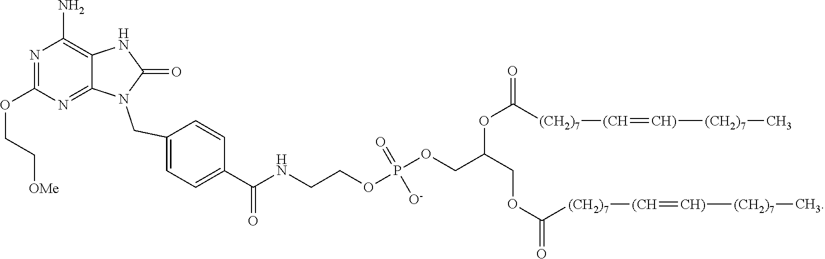

36. The formulation of any one of claims 23 to 33 wherein the compound of Formula (I) is

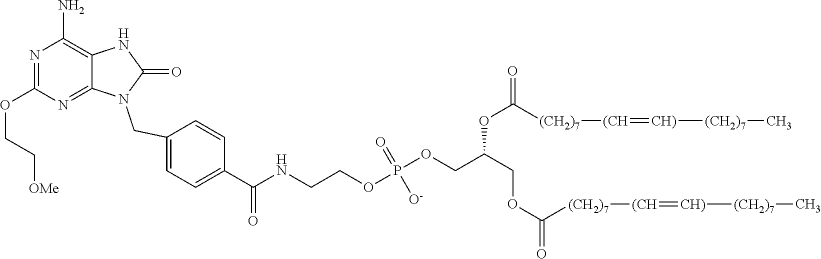

37. The formulation of any one of claims 17 to 36 wherein the TLR4 agonist comprises formula (II): 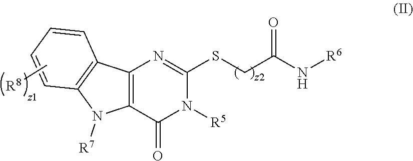
wherein zI is an integer from 0 to 4, wherein z2 is an integer from 0 to 5, wherein R⁵ is substituted or unsubstituted cycloalkyl, substituted or unsubstituted heterocycloalkyl, substituted or unsubstituted aryl, or substituted or unsubstituted heteroaryl, wherein R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R⁷ is hydrogen, or substituted or unsubstituted alkyl, and wherein each R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

38. The formulation of claim 37 wherein z2 is 1, 2 or 3.

39. The formulation of claim 37 or 38 wherein z1 is 1 or 2.

40. The formulation of claim 37 or 38 wherein z1 is 0.

41. The formulation of any one of claims 37 to 40 wherein R⁵ is substituted or unsubstituted aryl.

42. The formulation of any one of claims 37 to 41 wherein R⁶ is substituted or unsubstituted cycloalkyl.

43. The formulation of any one of claims 37 to 42 wherein R⁷ is substituted or unsubstituted alkyl.

44. The formulation of any one of claims 37 to 39 or 40 to 43 wherein z1=1 and R⁸ is a substituted or unsubstituted aryl or a substituted or unsubstituted heteroaryl.
